SEQUENCE LISTING (105 sequences)

SEQ ID NO: 1 - RNA, 22 nt, Mus sp.
  microRNA (up)
aaugcacccgggcaaggauuug

SEQ ID NO: 2 - RNA, 22 nt, Mus sp.
  microRNA (up)
uccgucucaguuacuuuauagc

SEQ ID NO: 3 - RNA, 21 nt, Mus sp.
  microRNA (up)
acuggacuuggagucagaagg

SEQ ID NO: 4 - RNA, 22 nt, Mus sp.
  microRNA (up)
acccgucccguucguccccgga

SEQ ID NO: 5 - RNA, 23 nt, Mus sp.
  microRNA (up)
ucucacacagaaaucgcacccgu

SEQ ID NO: 6 - RNA, 22 nt, Mus sp.
  microRNA (up)
ucagugcacuacagaacuuugu

SEQ ID NO: 7 - RNA, 22 nt, Mus sp.
  microRNA (up)
agaucgaccguguuauauucgc

SEQ ID NO: 8 - RNA, 21 nt, Mus sp.
  microRNA (up)
aauauaacacagauggccugu

SEQ ID NO: 9 - RNA, 21 nt, Mus sp.
  microRNA (up)
ugucuugcaggccgucaugca

SEQ ID NO: 10 - RNA, 22 nt, Mus sp.
  microRNA (up)
ucagugcaucacagaacuuugu

SEQ ID NO: 11 - RNA, 22 nt, Mus sp.
  microRNA (up)
uaacagucuccagucacggcca

SEQ ID NO: 12 - RNA, 22 nt, Mus sp.
  microRNA (up)
uauggcacugguagaauucacu

SEQ ID NO: 13 - RNA, 21 nt, Mus sp.
  mciroRNA (up)
uaguagaccguauagcguacg

SEQ ID NO: 14 - RNA, 22 nt, Mus sp.
  microRNA (up)
ucggauccgucugagcuuggcu

SEQ ID NO: 15 - RNA, 23 nt, Mus sp.
  microRNA (up)
accuuggcucuagacugcuuacu

SEQ ID NO: 16 - RNA, 22 nt, Mus sp.
  microRNA (up)
ugagaacugaauuccauaggcu

SEQ ID NO: 17 - RNA, 23 nt, Mus sp.
  mciroRNA (down)
aacauucaacgcugucggugagu

SEQ ID NO: 18 - RNA, 23 nt, Mus sp.
  microRNA (down)
uacccuguagauccgaauuugug

SEQ ID NO: 19 - RNA, 23 nt, Mus sp.
  microRNA (down)
aacauucauugcugucggugggu

SEQ ID NO: 20 - RNA, 21 nt, Mus sp.
  microRNA (down)
aauggcgccacuaggguugug

SEQ ID NO: 21 - RNA, 22 nt, Mus sp.
  microRNA (down)
ugagaacugaauuccauggguu

SEQ ID NO: 22 - RNA, 21 nt, Mus sp.
  microRNA (down)
cuagacugaggcuccuugagg

SEQ ID NO: 23 - RNA, 21 nt, Mus sp.
  microRNA (down)
uagcagcacagaaauauuggc

SEQ ID NO: 24 - RNA, 22 nt, Mus sp.
  microRNA (down)
gaguauuguuuccacugccugg

SEQ ID NO: 25 - RNA, 22 nt, Mus sp.
  microRNA (down)
gugaaauguuuaggaccacuag

SEQ ID NO: 26 - RNA, 21 nt, Mus sp.
  microRNA (down)
ccguccugagguuguugagcu

SEQ ID NO: 27 - RNA, 22 nt, Mus sp.
  microRNA (down)
acaggcugucugaucccacggu

SEQ ID NO: 28 - RNA, 22 nt, Mus sp.
  microRNA (down)
guaaacauccgacugaaagcuc

SEQ ID NO: 29 - protein, 5 aa, Artificial Sequence
  peptide specific for lung - Seq ID No. 1 WO 2015/018860
GHGYF

SEQ ID NO: 30 - protein, 7 aa, Artificial Sequence
  lung specifice peptide - Seq ID No. 2 WO 2015/018860
ESGHGYF

SEQ ID NO: 31 - protein, 744 aa, Artificial Sequence
  Seq ID No. 9 WO 2015/018860
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGESGHGYFAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - DNA, 1774 bp, Artificial Sequence
  CMV-mir181a-scAAV Double stranded AAV vector genome for simultaneous expression of a cDNA (eGFP) and a miRNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaaacattcaacgctgtcggtgagttagtgaagccacagatgtaaccatcgaccgttgattgtaccgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 1719 bp, Artificial Sequence
  CMV-mir181a-mir181b-mir10a-scAAV Double stranded AAV vector genome for simultaneous expression of three miRNAs
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaaggcgcgcctcgacttcttaacccaacagaaggctcgagaaggtatattgctgttgacagtgagcgaaacattcaacgctgtcggtgagttagtgaagccacagatgtaaccatcgaccgttgattgtaccgtgcctactgcctcggacttcaaggggctagaattcgatcgacttcttaacccaacagaaggctcgagaaggtatattgctgttgacagtgagcgaaacattcattgctgtcggtgggtttagtgaagccacagatgtactcactgatcaatgaatgcaaagtgcctactgcctcggacttcaaggggctagaattcgatcgacttcttaacccaacagaaggctcgagaaggtatattgctgttgacagtgagcgataccctgtagatccgaatttgtgtagtgaagccacagatgtacaaattcgtatctaggggaatagtgcctactgcctcggacttcaaggggctagaattcgagaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggagaacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - RNA, 22 nt, Homo sapiens
  microRNA (up)
aaugcacccgggcaaggauucu

SEQ ID NO: 35 - RNA, 22 nt, Homo sapiens
  miRNA (up)
acuggacuuggagucagaaggc

SEQ ID NO: 36 - RNA, 21 nt, Homo sapiens
  miRNA (up)
uaacagucuccagucacggcc

SEQ ID NO: 37 - RNA, 21 nt, Homo sapiens
  miRNA (down)
cuagacugaagcuccuugagg

SEQ ID NO: 38 - RNA, 21 nt, Homo sapiens
  miRNA (down)
uagcagcacagaaauauuggc

SEQ ID NO: 39 - RNA, 23 nt, Homo sapiens
  miRNA (down)
gggguauuguuuccgcugccagg

SEQ ID NO: 40 - DNA, 159 bp, Artificial Sequence
  >mir-10a-5p 23 nt, miR-E backbone, Passenger position
tcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcga

SEQ ID NO: 41 - DNA, 159 bp, Artificial Sequence
  >mir-10a-5p 23 nt, miR-E backbone, Guide position
tcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcga

SEQ ID NO: 42 - DNA, 157 bp, Artificial Sequence
  >mir-10a-5p 22 nt, miR-E backbone, Passenger position
tcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcga

SEQ ID NO: 43 - DNA, 157 bp, Artificial Sequence
  >mir-10a-5p 22 nt, miR-E backbone, Guide position
tcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcga

SEQ ID NO: 44 - DNA, 184 bp, Artificial Sequence
  >mir-10a-5p, natural pre-miRNA in miR-E backbone, Human(hsa-mir-10a MI0000266)
tcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcga

SEQ ID NO: 45 - DNA, 184 bp, Artificial Sequence
  >mir-10a-5p, natural pre-miRNA in miR-E backbone, Mouse(mmu-mir-10a MI0000685)
tcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcga

SEQ ID NO: 46 - DNA, 159 bp, Artificial Sequence
  >mir-181a-5p 23 nt, miR-E backbone, Passenger position
tcgacttcttaacccaacagaaggctcgagaaggtatattgctgttgacagtgagcgaacattcaacgctgtcggtgagttagtgaagccacagatgtaactcaccgacagcgttgaatgtgtgcctactgcctcggacttcaaggggctagaattcga

SEQ ID NO: 47 - DNA, 159 bp, Artificial Sequence
  >mir-181a-5p 23 nt, miR-E backbone, Guide position
tcgacttcttaacccaacagaaggctcgagaaggtatattgctgttgacagtgagcgcctcaccgacagcgttgaatgtttagtgaagccacagatgtaaacattcaacgctgtcggtgagttgcctactgcctcggacttcaaggggctagaattcga

SEQ ID NO: 48 - DNA, 157 bp, Artificial Sequence
  >mir-181a-5p 22 nt, miR-E backbone, Passenger position
tcgacttcttaacccaacagaaggctcgagaaggtatattgctgttgacagtgagcgaacattcaacgctgtcggtgagtagtgaagccacagatgtactcaccgacagcgttgaatgtgtgcctactgcctcggacttcaaggggctagaattcga

SEQ ID NO: 49 - DNA, 157 bp, Artificial Sequence
  >mir-181a-5p 22 nt, miR-E backbone, Guide position
tcgacttcttaacccaacagaaggctcgagaaggtatattgctgttgacagtgagcgatcaccgacagcgttgaatgtttagtgaagccacagatgtaaacattcaacgctgtcggtgagtgcctactgcctcggacttcaaggggctagaattcga

SEQ ID NO: 50 - DNA, 184 bp, Artificial Sequence
  >mir-181a-5p, natural pre-miRNA in miR-E backbone, Huma (hsa-mir-181a-1 MI0000289)
tcgacttcttaacccaacagaaggctcgagaaggtatattgctgttgtgagttttgaggttgcttcagtgaacattcaacgctgtcggtgagtttggaattaaaatcaaaaccatcgaccgttgattgtaccctatggctaaccatcatctactccactcggacttcaaggggctagaattcga

SEQ ID NO: 51 - DNA, 161 bp, Artificial Sequence
  >mir-181a-5p, natural pre-miRNA in miR-E back bone, Mouse (mmu-mir-181a-1 MI0000697)
tcgacttcttaacccaacagaaggctcgagaaggtatattgctgttgggttgcttcagtgaacattcaacgctgtcggtgagtttggaattcaaataaaaaccatcgaccgttgattgtaccctatagctaaccctcggacttcaaggggctagaattcga

SEQ ID NO: 52 - DNA, 159 bp, Artificial Sequence
  >mir-181b-5p 23 nt, miR-E backbone, Passenger position
tcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcga

SEQ ID NO: 53 - DNA, 159 bp, Artificial Sequence
  >mir-181b-5p 23 nt, miR-E backbone, Guide position
tcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcga

SEQ ID NO: 54 - DNA, 157 bp, Artificial Sequence
  >mir-181b-5p 22 nt, miR-E backbone, Passenger position
tcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcga

SEQ ID NO: 55 - DNA, 157 bp, Artificial Sequence
  >mir-181b-5p 22 nt, miR-E backbone, Guide position
tcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcga

SEQ ID NO: 56 - DNA, 184 bp, Artificial Sequence
  >mir-181b-5p, natural pre-miRNA in miR-E back bone, Human (hsa-mir-181b-1 MI0000270)
tcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcga

SEQ ID NO: 57 - DNA, 154 bp, Artificial Sequence
  >mir-181b-5p, natural pre-miRNA in miR-E back bone, Mouse (mmu-mir-181b-1 MI0000723)
tcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcga

SEQ ID NO: 58 - DNA, 159 bp, Artificial Sequence
  >mir-212-5p 23 nt, miR-E backbone, Passenger position
tcgacttcttaacccaacagaaggctcgagaaggtatattgctgttgacagtgagcgaccttggctctagactgcttacttagtgaagccacagatgtaagtaagcagtctagagccaaggctgcctactgcctcggacttcaaggggctagaattcga

SEQ ID NO: 59 - DNA, 159 bp, Artificial Sequence
  >mir-212-5p 23 nt, miR-E backbone, Guide position
tcgacttcttaacccaacagaaggctcgagaaggtatattgctgttgacagtgagcgcgtaagcagtctagagccaaggttagtgaagccacagatgtaaccttggctctagactgcttacttgcctactgcctcggacttcaaggggctagaattcga

SEQ ID NO: 60 - DNA, 157 bp, Artificial Sequence
  >mir-212-5p 22 nt, miR-E backbone, Passenger position
tcgacttcttaacccaacagaaggctcgagaaggtatattgctgttgacagtgagcgaccttggctctagactgcttactagtgaagccacagatgtagtaagcagtctagagccaaggctgcctactgcctcggacttcaaggggctagaattcga

SEQ ID NO: 61 - DNA, 157 bp, Artificial Sequence
  >mir-212-5p 22 nt, miR-E backbone, Guide position
tcgacttcttaacccaacagaaggctcgagaaggtatattgctgttgacagtgagcgataagcagtctagagccaaggttagtgaagccacagatgtaaccttggctctagactgcttactgcctactgcctcggacttcaaggggctagaattcga

SEQ ID NO: 62 - DNA, 184 bp, Artificial Sequence
  >mir-212-5p, natural pre-miRNA in miR-E back bone, Human (hsa-mir-212 MI0000288)
tcgacttcttaacccaacagaaggctcgagaaggtatattgctgttgcggggcaccccgcccggacagcgcgccggcaccttggctctagactgcttactgcccgggccgccctcagtaacagtctccagtcacggccaccgacgcctggccccgccctcggacttcaaggggctagaattcga

SEQ ID NO: 63 - DNA, 165 bp, Artificial Sequence
  >mir-212-5p, natural pre-miRNA in miR-E back bone, Mouse (mmu-mir-212 MI0000696)
tcgacttcttaacccaacagaaggctcgagaaggtatattgctgttggggcagcgcgccggcaccttggctctagactgcttactgcccgggccgccttcagtaacagtctccagtcacggccaccgacgcctggcccctcggacttcaaggggctagaattcga

SEQ ID NO: 64 - DNA, 1773 bp, Artificial Sequence
  >scAAV-CMV-eGFP-mir181b-5p(23 nt in miR-E back bone)-SV40pA, Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 1773 bp, Artificial Sequence
  >scAAV-CMV-eGFP-mir181b-5p(23 nt in miR-E back bone)-SV40pA, Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 66 - DNA, 1771 bp, Artificial Sequence
  >scAAV-CMV-eGFP-mir181b-5p(22 nt in miR-E back bone)-SV40pA, Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 1771 bp, Artificial Sequence
  >scAAV-CMV-eGFP-mir181b-5p(22 nt in miR-E back bone)-SV40pA, Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 68 - DNA, 1798 bp, Artificial Sequence
  >scAAV-CMV-eGFP-mir181b-5p(natural pre-miRNA, human)-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 69 - DNA, 1768 bp, Artificial Sequence
  >scAAV-CMV-eGFP-mir181b-5p(natural pre-miRNA, mouse)-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 70 - DNA, 2091 bp, Artificial Sequence
  >scAAV-CMV-eGFP-mir-181a-mir181b-mir10a(all 23 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcaacgctgtcggtgagttagtgaagccacagatgtaactcaccgacagcgttgaatgtgtgcctactgcctcggacttcaaggggctagaattcgatcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 71 - DNA, 2091 bp, Artificial Sequence
  >scAAV-CMV-eGFP-mir-181a-mir181b-mir10a(all 23 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgcctcaccgacagcgttgaatgtttagtgaagccacagatgtaaacattcaacgctgtcggtgagttgcctactgcctcggacttcaaggggctagaattcgatcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgatcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 72 - DNA, 2085 bp, Artificial Sequence
  >scAAV-CMV-eGFP-mir-181a-mir181b-mir10a(all 22 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaacattcaacgctgtcggtgagtagtgaagccacagatgtactcaccgacagcgttgaatgtgtgcctactgcctcggacttcaaggggctagaattcgatcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 73 - DNA, 2085 bp, Artificial Sequence
  >scAAV-CMV-eGFP-mir-181a-mir181b-mir10a(all 22 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgatcaccgacagcgttgaatgtttagtgaagccacagatgtaaacattcaacgctgtcggtgagtgcctactgcctcggacttcaaggggctagaattcgatcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgatcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 74 - DNA, 2091 bp, Artificial Sequence
  >scAAV-CMV-eGFP-mir-212-5p-mir181b-mir10a(all 23 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccttggctctagactgcttacttagtgaagccacagatgtaagtaagcagtctagagccaaggctgcctactgcctcggacttcaaggggctagaattcgatcgacttcttaacccaacagaaggctcgagaaggtatattgctgttgacagtgagcgaacattcattgctgtcggtgggttagtgaagccacagatgtaacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgtgtagtgaagccacagatgtacacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 75 - DNA, 2091 bp, Artificial Sequence
  >scAAV-CMV-eGFP-mir-212-5p-mir181b-mir10a(all 23 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgcgtaagcagtctagagccaaggttagtgaagccacagatgtaaccttggctctagactgcttacttgcctactgcctcggacttcaaggggctagaattcgatcgacttcttaacccaacagaaggctcgagaaggtatattgctgttgacagtgagcgccccaccgacagcaatgaatgtttagtgaagccacagatgtaaacattcattgctgtcggtgggttgcctactgcctcggacttcaaggggctagaattcgatcgacttcttaacccaacagaaggctcgagaaggtatattgctgttgacagtgagcgaacaaattcggatctacagggtatagtgaagccacagatgtataccctgtagatccgaatttgtgtgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 76 - DNA, 2085 bp, Artificial Sequence
  >scAAV-CMV-eGFP-mir-212-5p-mir181b-mir10a(all 22 nt in miR-E backbone), Passenger position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgaccttggctctagactgcttactagtgaagccacagatgtagtaagcagtctagagccaaggctgcctactgcctcggacttcaaggggctagaattcgatcgacttcttaacccaacagaaggctcgagaaggtatattgctgttgacagtgagcgaacattcattgctgtcggtgggtagtgaagccacagatgtacccaccgacagcaatgaatgtgtgcctactgcctcggacttcaaggggctagaattcgatcgacttcttaacccaacagaaggctcgagaaggtatattgctgttgacagtgagcgtaccctgtagatccgaatttgttagtgaagccacagatgtaacaaattcggatctacagggtctgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 77 - DNA, 2085 bp, Artificial Sequence
  >scAAV-CMV-eGFP-mir-212-5p-mir181b-mir10a(all 22 nt in miR-E backbone), Guide position
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgacagtgagcgataagcagtctagagccaaggttagtgaagccacagatgtaaccttggctctagactgcttactgcctactgcctcggacttcaaggggctagaattcgatcgacttcttaacccaacagaaggctcgagaaggtatattgctgttgacagtgagcgaccaccgacagcaatgaatgtttagtgaagccacagatgtaaacattcattgctgtcggtgggtgcctactgcctcggacttcaaggggctagaattcgatcgacttcttaacccaacagaaggctcgagaaggtatattgctgttgacagtgagcgccaaattcggatctacagggtatagtgaagccacagatgtataccctgtagatccgaatttgttgcctactgcctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 2166 bp, Artificial Sequence
  >scAAV-CMV-eGFP-mir-181a-mir181b-mir10a(natural pre-miRNAs in miR-E backbone), Human
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgtgagttttgaggttgcttcagtgaacattcaacgctgtcggtgagtttggaattaaaatcaaaaccatcgaccgttgattgtaccctatggctaaccatcatctactccactcggacttcaaggggctagaattcgatcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgatcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 79 - DNA, 2113 bp, Artificial Sequence
  >scAAV-CMV-eGFP-mir-181a-mir181b-mir10a(natural pre-miRNAs in miR-E backbone), Mouse
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgggttgcttcagtgaacattcaacgctgtcggtgagtttggaattcaaataaaaaccatcgaccgttgattgtaccctatagctaaccctcggacttcaaggggctagaattcgatcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgatcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 80 - DNA, 2166 bp, Artificial Sequence
  >scAAV-CMV-eGFP-mir-212-5p-mir181b-mir10a(natur al pre-miRNAs in miR-E backbone), Human
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttgcggggcaccccgcccggacagcgcgccggcaccttggctctagactgcttactgcccgggccgccctcagtaacagtctccagtcacggccaccgacgcctggccccgccctcggacttcaaggggctagaattcgatcgacttcttaacccaacagaaggctcgagaaggtatattgctgttgcctgtgcagagattattttttaaaaggtcacaatcaacattcattgctgtcggtgggttgaactgtgtggacaagctcactgaacaatgaatgcaactgtggccccgcttctcggacttcaaggggctagaattcgatcgacttcttaacccaacagaaggctcgagaaggtatattgctgttggatctgtctgtcttctgtatataccctgtagatccgaatttgtgtaaggaattttgtggtcacaaattcgtatctaggggaatatgtagttgacataaacactccgctctctcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 81 - DNA, 2117 bp, Artificial Sequence
  >scAAV-CMV-eGFP-mir-212-5p-mir181b-mir10a(natur al pre-miRNAs in miR-E backbone), Mouse
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagaggtaccttaattaagccaccatggtgtccaagggcgaggaactgttcaccggcgtggtgcccatcctggtggaactggatggcgacgtgaacggccacaagttcagcgtgtccggcgagggcgaaggcgacgccacatatggcaagctgaccctgaagttcatctgcaccaccggcaagctgcccgtgccttggcctaccctcgtgaccacactgacctacggcgtgcagtgcttcagcagataccccgaccatatgaagcagcacgacttcttcaagagcgccatgcccgagggctacgtgcaggaacggaccatcttctttaaggacgacggcaactacaagaccagggccgaagtgaagttcgagggcgacaccctcgtgaaccggatcgagctgaagggcatcgacttcaaagaggacggcaacatcctgggccacaagctggagtacaactacaacagccacaacgtgtacatcatggccgacaagcagaaaaacggcatcaaagtgaacttcaagatccggcacaacatcgaggacggctccgtgcagctggccgaccactaccagcagaacacccccatcggagatggccccgtgctgctgcccgacaaccactacctgagcacacagagcgccctgagcaaggaccccaacgagaagcgggaccacatggtgctgctggaatttgtgaccgccgctggcatcaccctgggcatggacgagctgtacaaatgaggcgcgcctcgacttcttaacccaacagaaggctcgagaaggtatattgctgttggggcagcgcgccggcaccttggctctagactgcttactgcccgggccgccttcagtaacagtctccagtcacggccaccgacgcctggcccctcggacttcaaggggctagaattcgatcgacttcttaacccaacagaaggctcgagaaggtatattgctgttgaggtcacaatcaacattcattgctgtcggtgggttgaactgtgtagaaaagctcactgaacaatgaatgcaactgtggccctcggacttcaaggggctagaattcgatcgacttcttaacccaacagaaggctcgagaaggtatattgctgttggacctgtctgtcttctgtatataccctgtagatccgaatttgtgtaaggaattttgtggtcacaaattcgtatctaggggaatatgtagttgacataaacactccgctcactcggacttcaaggggctagaattcgagacttgtttattgcagcttataatggttacaaataaagcaatagcatcacaaatttcacaaataaagcatttttttcactgcattctagttgtggtttgtccaaactcatcaatgtatcttaacgcggccgagatctccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 82 - protein, 736 aa, Artificial Sequence
  AAV6.2 VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 83 - DNA, 157 bp, Artificial Sequence
  mir- Ren713, neutral control, mirE backbone
tcgacttcttaacccaacagaaggctcgagaaggtatattgctgttgacagtgagcgcaggaattataatgcttatctatagtgaagccacagatgtatagataagcattataattcctatgcctactgcctcggacttcaaggggctagaattcga

SEQ ID NO: 84 - DNA, 182 bp, Artificial Sequence
  mir-181a stem-loop, miR-E context
tcgacttcttaacccaacagaaggctcgagaaggtatattgctgtttgagttttgaggttgcttcagtgaacattcaacgctgtcggtgagtttggaattaaaatcaaaaccatcgaccgttgattgtaccctatggctaaccatcatctactccatcggacttcaaggggctagaattcga

SEQ ID NO: 85 - DNA, 182 bp, Artificial Sequence
  mir-212 stem-loop, miR-E context
tcgacttcttaacccaacagaaggctcgagaaggtatattgctgttcggggcaccccgcccggacagcgcgccggcaccttggctctagactgcttactgcccgggccgccctcagtaacagtctccagtcacggccaccgacgcctggccccgcctcggacttcaaggggctagaattcga

SEQ ID NO: 86 - DNA, 157 bp, Artificial Sequence
  mir-29a-3p, miR-E backbone
tcgacttcttaacccaacagaaggctcgagaaggtatattgctgttgacagtgagcgcaaccgatttcagatggtgctatagtgaagccacagatgtatagcaccatctgaaatcggttatgcctactgcctcggacttcaaggggctagaattcga

SEQ ID NO: 87 - DNA, 159 bp, Artificial Sequence
  mir-181a stem-loop, miR-E context, Mouse
tcgacttcttaacccaacagaaggctcgagaaggtatattgctgttggttgcttcagtgaacattcaacgctgtcggtgagtttggaattcaaataaaaaccatcgaccgttgattgtaccctatagctaacctcggacttcaaggggctagaattcga

SEQ ID NO: 88 - DNA, 163 bp, Artificial Sequence
  mir-212 stem-loop, miR-E context, Mouse
tcgacttcttaacccaacagaaggctcgagaaggtatattgctgttgggcagcgcgccggcaccttggctctagactgcttactgcccgggccgccttcagtaacagtctccagtcacggccaccgacgcctggccctcggacttcaaggggctagaattcga

SEQ ID NO: 89 - DNA, 136 bp, Artificial Sequence
  mir-29a stem-loop, miR-E context
tcgacttcttaacccaacagaaggctcgagaaggtatattgctgttatgactgatttcttttggtgttcagagtcaatataattttctagcaccatctgaaatcggttattcggacttcaaggggctagaattcga

SEQ ID NO: 90 - DNA, 138 bp, Artificial Sequence
  mir-29a, natural pre-miRNA in miR-E backbone
tcgacttcttaacccaacagaaggctcgagaaggtatattgctgttgatgactgatttcttttggtgttcagagtcaatataattttctagcaccatctgaaatcggttatctcggacttcaaggggctagaattcga

SEQ ID NO: 91 - DNA, 3846 bp, Artificial Sequence (plasmid)
  >#2760_pAAVsc_CMV-miR212-5p\22nt\in mir-E back bone, circular
  misc_feature 1: /note=“first nucleotide ITR”
  misc_feature 150..1131: /note=“CMV-miR-212-polyA expression cassete”
  misc_feature 753..909: /note=“see Seq ID NO 61”
  misc_feature 1249: /note=“last nucleotide ITR”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggtaccggcgcgcctcgacttcttaacccaacagaaggctcgagaaggtatattgctgttgacagtgagcgataagcagtctagagccaaggttagtgaagccacagatgtaaccttggctctagactgcttactgcctactgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcagatctccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 92 - RNA, 22 nt, Artificial Sequence
  hsa miR-29a-3p
uagcaccaucugaaaucgguua

SEQ ID NO: 93 - DNA, 529 bp, Artificial Sequence
  >mir-29a\stem\loop-mir181a\stem\loop-bGH\pA
tcgacttcttaacccaacagaaggctcgagaaggtatattgctgttatgactgatttcttttggtgttcagagtcaatataattttctagcaccatctgaaatcggttattcggacttcaaggggctagaattcgatcgacttcttaacccaacagaaggctcgagaaggtatattgctgtttgagttttgaggttgcttcagtgaacattcaacgctgtcggtgagtttggaattaaaatcaaaaccatcgaccgttgattgtaccctatggctaaccatcatctactccatcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 94 - DNA, 529 bp, Artificial Sequence
  mir-29a\stem\loop-mir212\stem\loop-bGH\pA
tcgacttcttaacccaacagaaggctcgagaaggtatattgctgttatgactgatttcttttggtgttcagagtcaatataattttctagcaccatctgaaatcggttattcggacttcaaggggctagaattcgatcgacttcttaacccaacagaaggctcgagaaggtatattgctgttcggggcaccccgcccggacagcgcgccggcaccttggctctagactgcttactgcccgggccgccctcagtaacagtctccagtcacggccaccgacgcctggccccgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 95 - DNA, 527 bp, Artificial Sequence
  mir-29a-3p-mir-181a-5p\23\nt,\miR-E\backbone,\ Guide\position,\bGH\pA
tcgacttcttaacccaacagaaggctcgagaaggtatattgctgttgacagtgagcgcaaccgatttcagatggtgctatagtgaagccacagatgtatagcaccatctgaaatcggttatgcctactgcctcggacttcaaggggctagaattcgatcgacttcttaacccaacagaaggctcgagaaggtatattgctgttgacagtgagcgcctcaccgacagcgttgaatgtttagtgaagccacagatgtaaacattcaacgctgtcggtgagttgcctactgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 96 - DNA, 525 bp, Artificial Sequence
  mir-29a-3p-mir-181a-5p\22\nt,\miR-E\backbone,\ Guide\position,\bGH\pA
tcgacttcttaacccaacagaaggctcgagaaggtatattgctgttgacagtgagcgcaaccgatttcagatggtgctatagtgaagccacagatgtatagcaccatctgaaatcggttatgcctactgcctcggacttcaaggggctagaattcgatcgacttcttaacccaacagaaggctcgagaaggtatattgctgttgacagtgagcgatcaccgacagcgttgaatgtttagtgaagccacagatgtaaacattcaacgctgtcggtgagtgcctactgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 97 - DNA, 527 bp, Artificial Sequence
  mir-29a-3p-mir-212-5p\23\nt,\miR-E\backbone,\ Guide\position,\bGH\ pA
tcgacttcttaacccaacagaaggctcgagaaggtatattgctgttgacagtgagcgcaaccgatttcagatggtgctatagtgaagccacagatgtatagcaccatctgaaatcggttatgcctactgcctcggacttcaaggggctagaattcgatcgacttcttaacccaacagaaggctcgagaaggtatattgctgttgacagtgagcgcgtaagcagtctagagccaaggttagtgaagccacagatgtaaccttggctctagactgcttacttgcctactgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 98 - DNA, 525 bp, Artificial Sequence
  mir-29a-3p-mir-212-5p\22\nt,\miR-E\backbone,\ Guide\position,\bGH\ pA
tcgacttcttaacccaacagaaggctcgagaaggtatattgctgttgacagtgagcgcaaccgatttcagatggtgctatagtgaagccacagatgtatagcaccatctgaaatcggttatgcctactgcctcggacttcaaggggctagaattcgatcgacttcttaacccaacagaaggctcgagaaggtatattgctgttgacagtgagcgataagcagtctagagccaaggttagtgaagccacagatgtaaccttggctctagactgcttactgcctactgcctcggacttcaaggggctagaattcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 99 - RNA, 22 nt, Artificial Sequence
  hsa-mir-212-5p, 22 nt
accuuggcucuagacugcuuac

SEQ ID NO: 100 - RNA, 22 nt, Artificial Sequence
  hsa-miR-181a-5p, 22nt
aacauucaacgcugucggugag

SEQ ID NO: 101 - RNA, 22 nt, Artificial Sequence
  miRNA mimetic
uagcaccaucugaaaucgguua

SEQ ID NO: 102 - RNA, 23 nt, Artificial Sequence
  miRNA mimetic
aacauucaacgcugucggugagu

SEQ ID NO: 103 - RNA, 23 nt, Artificial Sequence
  miRNA mimetic
accuuggcucuagacugcuuacu

SEQ ID NO: 104 - RNA, 21 nt, Artificial Sequence
  miRNA mimetic negative control
gauggcauucgaucaguucua

SEQ ID NO: 105 - RNA, 110 nt, Artificial Sequence
  natural pre-miRNA in miR-E backbone
ccugugcagagauuauuuuuuaaaaggucacaaucaacauucauugcugucgguggguugaacuguguggacaagcucacugaacaaugaaugcaacuguggccccgcuu

(19) United States
(12) Patent Application Publication
(10) Pub. No.: US 2024/0011033 A1
(43) Pub. Date: Jan. 11, 2024

(54) VIRAL VECTORS AND NUCLEIC ACIDS FOR USE IN THE TREATMENT OF ILD, PF-ILD AND IPF

(71) Applicant: BOEHRINGER INGELHEIM INTERNATIONAL GMBH, Ingelheim am Rhein (DE)

(72) Inventors: Sebastian Kreuz, Ingelheim am Rhein (DE); Ludger Ickenstein, Ingelheim am Rhein (DE); Marc Kaestle, Ingelheim am Rhein (DE); Stephan Klee, Ingelheim am Rhein (DE); Holger Klein, Ingelheim am Rhein (DE); Thorsten Lamla, Ingelheim am Rhein (DE); Benjamin Strobel, Ingelheim am Rhein (DE)

(21) Appl. No.: 18/251,597

(22) PCT Filed: Nov. 4, 2021
(86) PCT No.: PCT/EP2021/080622

§ 371 Date: May 3, 2023

(30) Foreign Application Priority Data
Nov. 4, 2020 (EP) 20205805.3

(51) Int. Cl. C12N 15/113; C12N 15/86; A61K 35/76; A61P 11/00

(52) U.S. Cl. CPC C12N 15/113; C12N 15/86; A61K 35/76; A61P 11/00; C12N 2310/141; C12N 2750/14143; C12N 2750/14122; C12N 2310/3231

(57) ABSTRACT

Viral vector comprising: a capsid and a packaged nucleic acid, wherein the nucleic acid either augments the miRNA downregulated in a Bleomycin-induced lung fibrosis model or in an AAV-TGFβ1-induced lung fibrosis model, or wherein the nucleic acid inhibits the miRNA up-regulated in a Bleomycin-induced lung fibrosis model or in an AAV-TGFβ1-induced lung fibrosis model.

### RELATED APPLICATIONS

This application is the U.S. National Stage of International Patent Application No. PCT/EP2021/080622, filed Nov. 4, 2021, which is hereby incorporated by reference in its entirety, and which claims priority to European Patent Application No. 20205805.3, filed Nov. 4, 2020.

### SEQUENCE LISTING

The sequences listed in the accompanying Sequence Listing are presented in accordance with 37 C.F.R. 1.822. The Sequence Listing is submitted as an ASCII computer readable text file, entitled “SequenceListing_58822-US.txt” created on Jul. 7, 2023, as 98,691 bytes, which is incorporated by reference herein.

### BACKGROUND OF THE INVENTION

The term interstitial lung disease (ILD) encompasses a large and heterogeneous group of over 200 pulmonary disorders, most of which are classified as rare. The major abnormality in ILDs is the disruption of the distal lung parenchyma resulting in impaired gas exchange and restrictive ventilatory defects. It is generally agreed that some form of injury of the alveolar epithelial cells initiates an inflammatory response coupled with repair mechanisms. The injury-repair process is reflected pathologically as inflammation, fibrosis or a combination of both. Irrespective of the underlying pathophysiology, the resulting alteration of the interstitial space leads to clinical symptoms such as dyspnoea and cough, and results in restrictive ventilatory and gas exchange deficits on pulmonary function testing (Schwartz M I et al., 2011). There is no universally accepted single classification of ILDs. They can generally be categorized based on their etiology (idiopathic or ILDs with known association or cause), clinical course (acute, subacute or chronic ILDs), and based on the main pathological features (inflammatory or fibrotic ILDs). Fibrotic ILDs can be subdivided into 3 groups based on their longitudinal disease behavior (Wells A U, 2004):
Intrinsically non-progressive, e.g. drug-induced lung disease after removal of the drug or some cases of hypersensitivity pneumonitis (HP) after removal of a trigger;
Progressive but stabilized by immunomodulation, e.g. some cases of connective tissue disease (CTD)-ILDs (Tashkin D P et al., 2006; Fischer A et al., 2013; Morisset J et al., 2017; Adegunsoye A et al., 2017);
Progressive despite treatment considered appropriate in individual ILDs, e.g. idiopathic pulmonary fibrosis (IPF).

While IPF is the best-known and prototypical form of a progressive fibrosing ILD (PFILD), there is a group of patients with different clinical ILD diagnoses other than IPF who develop a progressive fibrosing phenotype during the course of their disease. These patients demonstrate a number of similarities to patients with IPF, with their disease being defined by increasing extent of pulmonary fibrosis on imaging, declining lung function, worsening respiratory symptoms and quality of life despite management considered appropriate in individual ILDs, and, ultimately, early mortality (Flaherty K R et al., 2017; Wells A U et al., 2018; Cottin V et al., 2019; Kolb M et al., 2019). Similar to IPF, a decline in FVC is predictive of mortality in patients with these other fibrosing ILDs (Jegal Y et al., 2005; Solomon J J et al., 2016; Gimenez A, et al., 2017; Goh N S et al., 2017; Volkmann E R et al. 2019). There is a high unmet medical need, as no approved disease-modifying pharmacological therapies for patients with progressive fibrosing ILDs exist, except for patients with IPF. Along with their clinical similarities, progressive fibrosing ILDs share pathophysiological mechanisms that represent a common fibrotic response to tissue injury (see FIG. 3) (Thannickal V J et al., 2014; Bagnato G ate al., 2015; Wollin L et al., 2019; Luckhardt T R et al., 2015). These mechanisms are multifactorial and complex. The profibrotic and pro-proliferative milieu in the lung of ILD patients leads to an increase and proliferation of smooth muscle cells and endothelial cells. The resulting increase in muscularization of the distal pulmonary arteriole and increased capillary growth likely contributes to increased vascular resistance.

According to the scientific literature, ILDs that can be complicated by progressive fibrosis include, but are not limited to, idiopathic non-specific interstitial pneumonia (iNSIP) (Kim M Y et al., 2012), unclassifiable idiopathic interstitial pneumonia (IIP) (Guler S A et al., 2018), hypersensitivity pneumonitis (HP) (Sadeleer L J et al., 2019), autoimmune ILDs such as rheumatoid arthritis-associated ILD (RA-ILD) (Doyle T J & Dellaripa P F 2017) and SSc-ILD Guler S A et al, 2018), sarcoidosis (Walsh S L et al., 2014), and occupation associated lung disease (Khalil N et al., 2007). The etiology of progressive fibrosing ILDs like IPF is still unknown; however various irritants including smoking, occupational hazards, viral and bacterial infections as well as radiotherapy and chemotherapeutic agents (like e.g. Bleomycin) have been described as potential risk factors for the development of IPF. Due to changes in IPF diagnostic criteria over the past years, the prevalence of IPF varies considerably in the literature. According to recent data, the prevalence of IPF ranges from 14.0 to 63.0 cases per 100,000 while the incidence lies between 6.8 and 17.4 new annual cases per 100,000 (Ley B et al., 2013). IPF is usually diagnosed in elderly people with an average age of disease onset of 66 (Hopkins R B et al., 2016). After initial diagnosis IPF progresses rapidly with a mortality rate of approximately 60 percent within 3 to 5 years. In contrast to IPF, a variable portion of the patients with CTD (including e.g. rheumatoid arthritis (RA), Sjögren's syndrome and systemic sclerosis (SSc)) or sarcoidosis display a progressive fibrosing phenotype, with about 10-20% of RA patients, 9-24% of Sjögren's syndrome, >70% of SSc (Mathai S C and Danoff S K, 2016) and 20-25% of sarcoidosis patients (Spagnolo P et al., 2018) developing pulmonary fibrosis.

There are two main histopathological characteristics observed in PF-ILDs, namely non-specific interstitial pneumonia (NSIP) and usual interstitial pneumonitis (UIP). The histopathological hallmarks of IPF are UIP and progressive interstitial fibrosis caused by excessive extracellular matrix deposition. UIP is characterized by a heterogeneous appearance with areas of subpleural and paraseptal fibrosis alternating with areas of less affected or normal lung parenchyma. Areas of active fibrosis, so-called fibroblastic foci, are characterized by fibroblast accumulation and excessive collagen deposition. Fibroblastic foci are frequently located between the vascular endothelium and the alveolar epithelium, thereby causing disruption of lung architecture and formation of characteristic “honeycomb”-like structures. Clinical manifestations of IPF are dramatically compromised oxygen diffusion, progressive decline of lung function, cough and severe impairments in quality of life.

UIP is also one of the main histopathological hallmarks in RA-ILD and late-stage sarcoidosis; however, other CTDs, such as SSc or Sjögren's, are mainly characterized by non-specific interstitial pneumonia (NSIP).

NSIP is characterized by less spatial heterogeneity, i.e. pathological anomalies are rather uniformly spread across the lung. In the cellular NSIP subtype, histopathology is characterized by inflammatory cells, whereas in the more common fibrotic subtype, additional areas of pronounced fibrosis are evident. However, pathological manifestations can be diverse, thereby complicating correct diagnosis and differentiation from other types of fibrosis, such as UIP/IPF.

Due to the unknown disease cause of IPF, the knowledge regarding pathological mechanisms on the cellular and molecular level is still limited. However, recent advances in translational research using experimental disease models (in vitro and in vivo) for functional studies as well as tissue samples from IPF patients for genomics/proteomics analyses enabled valuable insights into key disease mechanisms. According to our current understanding, IPF is initiated through repeated alveolar epithelial cell (AEC) micro-injuries, which finally result in an uncontrolled and persistent wound healing response. In more detail, AEC damage induces an aberrant activation of neighboring epithelial cells, thereby is leading to the recruitment of immune cells and stem or progenitor cells to the sites of injury. By secreting various cytokines, chemokines and growth factors, infiltrating cells produce a pro-inflammatory environment, which finally results in the expansion and activation of fibroblasts. Under physiological conditions these so-called myofibroblasts produce extracellular matrix (ECM) components to stabilize and repair damaged tissue. Moreover, myofibroblasts contribute to tissue contraction and wound closure in later stages of the wound healing process via their inherent contractile function. In contrast to physiological wound healing, inflammation and ECM production are not self-limiting in IPF. As a consequence this leads to a continuous deposition of ECM, which finally results in progressive lung stiffening and the destruction of lung architecture. Indeed, ECM biomarkers can be used to determine the onset of the treatment of PF-ILD, see WO2017/207643. On the molecular level the pathogenesis of IPF is orchestrated by a multitude of pro-fibrotic mediators and signaling pathways. Besides TGFβ, which plays a central role in IPF due to its potent pro-fibrotic effects, tyrosine kinase signaling and elevation of various corresponding growth factors like e.g. platelet-derived growth factor (PDGF) and fibroblast growth factor (FGF) contribute to the pathogenesis of IPF.

In recent years several drugs have been clinically tested for the treatment of IPF. However, so far only two drugs, Pirfenidone (Esbriet®; Roche/Genentech) and Nintedanib (Ofev®; Boehringer Ingelheim), showed convincing therapeutic efficacy by slowing down disease progression as demonstrated by reduced rates of lung function decline. Despite these encouraging results, the medical need in IPF is still high and additional therapies with improved efficacy and ideally disease modifying potential are urgently needed. Nintedanib is also approved for the treatment of systemic sclerosis associated ILD as well as for chronic fibrosing interstitial lung disease with progressive phenotype other than IPF. In general, the current management of ILDs is centred on the suppression of inflammation with corticosteroid or immunomodulatory therapy. The latter is based on anecdotal reports and uncontrolled treatment responses in small case series with the use of azathioprine, cyclosporine, cyclophosphamide, mycophenolate mofetil, rituximab, and tacrolimus. Some ILDs, e.g. some cases of CTD-ILDs can be stabilized by immunomodulation (Tashkin D P et al., 2006; Fischer A et al. 2013; Morisset J et al., 2017; Adegunsoye A et al., 2017), others are progressive despite (pharmacological and/or non-pharmacological) treatment considered appropriate in individual ILDs (Wells AU 2004), again demonstrating a remaining high demand for innovative therapeutic approaches.

Pulmonary hypertension (PH) is one of the most frequent complications in ILDs, which could be an independent driver of early mortality (Galie N et al., 2015). PH is defined as a disease with elevated right ventricular systolic pressure (RVSP), right ventricular pressure overload and right atrial and ventricular dilatation (Smith et al. (2013), Am J Med Sci, 346(3):221-225).

Chronic, fibrotic silicosis belongs to the family of ILDs. It is caused by a chronic, recurrent inhalation to crystalline silica, damaging the epithelial cells in the alveolar space and activates macrophages to produce an inflammatory response. Both factors, lead to an activation of resident fibroblasts and the associated massive deposition of extra cellular matrix in these lung areas.

### FIELD OF THE INVENTION

Due to the plethora of pathways involved in the pathogenesis of IPF and other fibrosing ILDs, multi-target therapies aiming to simultaneously modulate various disease mechanisms are likely to be most effective. However, respective approaches are difficult to implement by classical pharmacological strategies using small molecule compounds (NCEs) or biologicals (NBEs) like e.g. monoclonal antibodies, since both modalities are typically designed to specifically inhibit or activate a single drug target or a small set of closely related molecules. To enable multi-targeted therapies for PF-ILDs, microRNAs (miRNAs) represent a novel and highly attractive target class based on their ability to control and fine-tune entire signaling pathways or cellular mechanisms under physiological and pathophysiological conditions by regulating mRNA expression levels of a specific set of target genes. miRNAs are small non-coding RNAs, which are transcribed as pre-cursor molecules (pri-miRNAs). Inside the nucleus pri-miRNAs undergo a first maturation step to produce so called pre-miRNAs, which are characterized by a smaller hairpin structure. Following nuclear export, pre-miRNAs undergo a second processing step mediated by the Dicer enzyme, thereby generating two single strands of fully maturated miRNAs of approximately 22 nucleotides in length. To exert their gene regulatory function, mature miRNAs are incorporated into the RNA Induced Silencing Complex (RISC) to enable binding to miRNA binding sites positioned within the 3′-UTR of target mRNAs. Upon binding, miRNAs induce destabilization and cleavage of target mRNAs and/or modulate gene expression by inhibition of protein translation of respective mRNAs. To date more than 2000 miRNAs have been discovered in humans, which potentially regulate up to 30% of the transcriptome (Hammond S M, 2015).

The present invention discloses the identification of miRNAs involved in the pathogenesis of fibrosing lung disease and methods for the treatment of lung diseases such as PF-ILD by functional modulation of respective miRNAs in ILD patients, preferably in PF-ILD patients, in particular IPF patients, using viral vectors, in particular an Adeno-associated virus (AAV). The present invention focusses on the treatment of humans though mammals of any kind, especially companion animal mammals, such as horses, dogs and cats are also within the realm of the invention.

### BRIEF SUMMARY OF THE INVENTION

Treatment of patients with moderate (Child Pugh B) and severe (Child Pugh C) hepatic impairment with Ofev is not recommended (see EPAR). Esbriet must not be used by patients already taking fluvoxamine (a medicine used to treat depression and obsessive compulsive disorder) or patients with severe liver or kidney problems (see EPAR). Thus, there is still a high medical need for PF-ILD patients, and in particular for IPF patients that have severe liver and kidney problems. It is an object of this invention to provide treatment alternatives. An alternative object of the invention is to provide treatment alternatives that might be eligible even for the patient group that cannot benefit from the existing therapies. While Esbriet and Ofev have shown convincing efficacy in clinical trials, also side effects are associated that potentially limit the options for a combined therapy of both drugs (see both EPARs). Thus, there is still a high medical need for ILD treatments, such as PF-ILD and in particular IPF treatments, with less side effects or at least with side effects different from those seen with Ofev or Esbriet, so that combined therapy with either Esbriet or Ofev may be viable option to increase the overall treatment efficacy. It is an alternative object of the invention to provide treatment alternatives with a different risk/benefit profile compared to the established treatment options, e.g. with lesser side effects or with different side is effects compared to the established treatment options. While Esbriet and Ofev are intended for oral, i.e. systemic use, there is still a need for a treatment option that can be administered by local administration or both via local and systemic routes. It is an alternative object of the invention to provide a treatment option that can be administered by local administration or both via local and systemic routes.

It is a further alternative object of the invention to provide
a therapy option for ILD, PF-ILD or IPF with a single or a limited number of administrations of the active ingredient and/or a
a therapy option for ILD, PF-ILD or IPF that addresses multiple aspects of the phenotype of ILD and/or IPF and/or a
a therapy option for ILD, PF-ILD or IPF that also at addresses multiple aspects of the phenotype of ILD and/or IPF and/or a
a therapy option of ILD, PF-ILD or IPF that has the potential for a beneficial effect in diseases that have a significant co-morbidity with of ILD, PF-ILD and/or IPF and/or
compositions of tool compounds that reduce one or more aspects of the phenotype of ILD, PF-ILD or IPF in animal models and cell models of ILD, PF-ILD or IPF.

The present invention relates in one aspect to therapeutic agents, i.e. viral vectors or miRNA mimetics, for the treatment of ILD in general, and PF-ILD and IPF in particular.

The viral vectors according to the invention stop or slow one or more aspects of the tissue transformation seen in ILD, preferably in PF-ILD and more preferably IPF, such as the ECM deposits, by modulating miRNA function and thus stop or slow the decline in forced vital capacity seen in these diseases (see WO2017/207643 and references). The viral vectors according to the invention may be administered to the patient via local (intranasal, intratracheal, inhalative) or systemic (intravenous) routes. Especially AAV vectors can target the lung quite efficiently, have a low antigenic potential and are thus particularly suitable also for systemic administration.

From a therapeutic perspective, miRNA function can be modulated by delivering miRNA mimetics to increase effects of endogenous miRNAs, which are downregulated under fibrotic conditions, or by delivering molecules to block miRNAs or to reduce their availability by so-called anti-miRs or miRNA sponges, thus inhibiting functionality of endogenous miRNAs, which are upregulated under pathological conditions.

Moreover, miRNAs described in the present invention, which are upregulated, might also exert protective functions as part of a natural anti-fibrotic response. However, this effect is apparently not sufficient to resolve the pathology on its own. Therefore, in specific cases, the delivery of a miRNA mimetic for a sequence which is already elevated under fibrotic conditions can potentially further enhance its anti-fibrotic effect, thereby offering an additional model for therapeutic interventions.

Based on the fact that miRNAs orchestrate the simultaneous regulation of multiple target genes, viral vector mediated modulation of miRNA function represents an attractive strategy to enable multi-targeted therapies by affecting different disease pathways. The lung-fibrosis associated miRNAs described in the present invention distinguish from previously identified miRNAs by modulating different sets of target genes, thereby offering potential for improved therapeutic efficacy.

In the present invention a set of miRNAs associated with lung fibrosis has been identified by in-depth characterization and computational analysis of two disease-relevant animal models, in particular, Bleomycin-induced lung injury, characterized by a patchy, acute inflammation-driven fibrotic phenotype and AAV-TGFβ1 induced fibrosis that is reminiscent of the more homogenous NSIP pattern. Longitudinal transcriptional profiles of miRNAs and mRNAs as well as functional data have been generated to enable the identification of disease-associated miRNAs. Additionally, high confidence miRNA-mRNA regulatory relationships have been built based on sequence and expression anti-correlation, allowing for characterization of miRNAs in the context of the disease models based on their target sets. To further substantiate these findings, synthetic RNA oligonucleotide mimetics of selected miRNA candidates (mir-29a-3p, mir-10a-5p, mir-181a-5p, mir-181b-5p, mir212-5p) were generated and used for transient transfection experiments in cellular fibrosis models in primary human lung fibroblasts, primary human bronchial airway epithelial cells and A549 cells. By investigating the effect of transiently transfected miRNAs on major aspects of TGFβ-induced fibrotic remodeling (inflammation, proliferation, fibroblast to myofibroblast transition (FMT), epithelial to mesenchymal transition (EMT)) the predicted anti-fibrotic effects of the selected miRNAs could be confirmed. Finally, to translate these findings into clinical applications, novel therapeutic approaches for fibrosing lung diseases to enable modulation of PF-ILD associated miRNAs by using viral gene delivery based on Adeno-associated virus (AAV) vectors are described.

The miRNA mimetics according to the invention stop or slow one or more aspects of the tissue transformation seen in ILDSs like PF-ILD and IPF, such as the ECM deposits, by modulating miRNA function and thus stop or slow the decline in forced vital capacity seen in these diseases (see WO2017/207643 and references). Compared to viral vectors according to the invention, they have a different profile of side effects, such as a potentially lower antigenicity, thereby potentially allowing multiple treatments without immunosuppressive combined treatment.

By conducting a longitudinal in depth analysis of two disease-relevant animal models, namely the Bleomycin- and the AAV-TGFβ1-induced lung fibrosis model in mice, a novel set of 28 miRNAs has been identified. To select the most relevant miRNAs, the inventors developed a hit selection strategy based on systematic correlation analyses between gene expression profiling data and key functional disease parameters. Under consideration of the chronic nature of PF-ILDs the inventors describe expression of miRNAs, anti-miRs or miRNA sponges by viral vectors especially those based on Adeno-associated virus (AAV) as a novel therapeutic concept to enable long lasting expression of therapeutic nucleic acids for functional modulation of fibrosis-associated miRNAs.

### BRIEF DESCRIPTION OF THE DRAWINGS

FIG. 1 illustrates the study design. A total of 130 C57Bl/6 mice either received NaCl, 1 mg/kg Bleomycin or 2.5×10¹¹ vector genomes (vg) of either AAV6.2-stuffer control or AAV6.2-CMV-TGFβ1 vector by intratracheal administration. At each readout and sampling (RS) time point illustrated in the scheme, lung function measurement was performed and the wet lung weight was determined. The left lung was then used for histological assessment of fibrosis development and the right lung was lysed for the isolation of total lung RNA. RNA was applied to next generation sequencing in order to profile gene expression changes correlating with disease manifestation.

FIG. 2 shows data on the functional characterization of lung pathology. Mice were treated as described in FIG. 1 and fibrosis development was monitored. (A) Masson trichrome-stained histological lung sections from day 21 after administration demonstrate fibrosis manifestation evident from alveolar septa thickening, increased extracellular matrix deposition and presence of immune cells. The lower panel of images shows 10× magnified details of the upper panel of micrographs. (B) An increase in wet lung weight in AAV-TGFβ1 and Bleomycin treated animals indicates increased ECM deposition, leading to (C) strong impairment of lung function in fibrotic animals. Mean+/−SD, **p<0.01, ***p<0.001, relative to respective control treatment.

FIG. 3 summarizes results from the gene expression analysis. By performing parallel mRNA- and miRNA-sequencing, up- and down-regulated mRNAs (A) and miRNAs (B) were identified in both models at every time point analyzed. Cut-off criteria for identification of differentially expressed genes: P adj. (FDR)≤0.05, abs(log 2FC)≥0.5 (FC≥1.414). (C) mRNAs showing differential expression exclusively in one of the models were separated from mRNAs that were differentially expressed in both models (commonly DE) at each time point and applied to KEGG pathway enrichment analysis. The data show enrichment for acute inflammation (“cytokine-cytokine receptor interaction”) at early time points in the Bleomycin model but not the AAV model, whereas enrichment for fibrosis development (“ECM receptor interaction”) was observed in a time-dependent fashion in both models.

FIG. 4 provides an overview of the filtering process applied for identification of fibrosis-associated miRNAs. In a first step miRNAs correlating (C) or anti-correlating (AC) with lung function and/or lung weight in at least one of the two models were identified. Subsequently, correlated and anti-correlated miRNAs were filtered for candidates showing differential gene expression. By definition miRNAs were regarded as differentially expressed when expression level changes (P adj. (FDR)≤0.05, abs(log 2FC)≥0.5; up- or downregulation) were observed in at least one of the animal models at one or more time points. In a final step filtered miRNAs were assessed with regard to species conservation. miRNAs showing sequence identity in the seed region and an alignment score of at least 20 for the mature miRNA sequence between mouse and human were regarded as homologs, whereas the remaining miRNAs were categorized as mouse-specific and thus non-conserved. Finally, the resulting hit list was hand-curated by e.g. eliminating candidates is with dissimilar or strongly fluctuating expression profiles, previously patented miRNAs and non-conserved upregulated miRNAs, because those could not be targeted in humans.

FIG. 5 A shows fibrosis-associated miRNAs identified by applying the filtering process as described in FIG. 4. Except for mmu-miR-30f and mmu-miR-7656-3p, for which no human homologs were identified, all miRNAs shown are species conserved (highly similar or identical). Mismatches to the human homolog are shown in bold face and underlined. Depicted sequences represent the processed and fully maturated miRNAs.

The closest human homologs of the mouse sequences that are highly similar (albeit not identical) are shown in FIG. 5 B.

The shown sequences are also compiled in a sequence listing. In case of contradictions between the sequence listing and FIGS. 5 A and B, FIG. 5 represents the authentic sequence.

FIG. 6 schematically illustrates the target prediction workflow. For the miRNA candidates listed in FIG. 5, mRNA targets were predicted by querying DIANA, MiRanda, PicTar, TargetScan and miRDB databases. mRNAs predicted by at least two out of five databases were considered and filtered further by the anticorrelation of expression between miRNA and mRNA measurements in the animal models. Predicted mRNAs whose longitudinal expression was anti-correlated (rho≤−0.6) with the expression of its corresponding miRNA were called putative targets. Subsequently, target lists were subjected to pathway enrichment analysis for functional characterization of the miRNA target spectrum.

FIG. 7 shows the characterization of miRNA function based on enrichment of predicted target sets. Predicted target sets for each miRNA underwent enrichment tests vs. reference gene sets from different sources. The table shows −log(p adj) of a subset of the selected set of miRNAs for a small subset of selected gene sets that are relevant in the context of pulmonary fibrosis. Higher values indicate stronger enrichment.

FIG. 8 describes vector designs to enable expression of miRNAs or miRNA targeting constructs. (A) Single miRNAs or combinations of miRNAs, which are downregulated under fibrotic conditions, can be expressed from vectors using Polymerase-II (Pol-II) or Polymerase-III (Pol-III) promoters. miRNA sequences can be expressed by using the natural backbone of a respective miRNA or embedded into a foreign miRNA backbone, thereby generating an artificial miRNA. In both cases miRNAs are expressed as precursor miRNAs (pri-miRNAs), which are processed inside the cell into mature miRNAs. Mechanistically, processed miRNAs selectively bind to miRNA binding sites positioned in the 3′-UTR of target genes thereby leading to reduced expression levels of fibrosis-associated genes via mRNA degradation and/or inhibition of protein translation. (B) Inhibition of endogenous miRNAs, which are upregulated under fibrotic conditions, can be achieved by expression of antisense-like molecules, so called anti-miRs. Respective sequences can be expressed from a shRNA backbone or from an artificial miRNA backbone by using Pol-II or Pol-III promoters. After intracellular processing, anti-miRs bind to pro-fibrotic target miRNAs, thereby blocking their functionality. (C) An alternative approach to inhibit pro-fibrotic miRNAs is the expression of mRNAs harboring miRNA-specific targeting sequences, so-called sponges. Upon expression using a Pol-II promoter, miRNA sponges lead to the sequestration of pro-fibrotic miRNAs, thereby inhibiting their pathological function.

FIG. 9 illustrates the generation of Adeno-associated virus (AAV) vectors for delivery of miRNA-expressing or miRNA-targeting constructs to the lung. Flanking of expression constructs by AAV inverted terminal repeats (ITRs) at the 5′- and the 3′-end enables packaging into AAV vectors. Various natural serotypes (AAV5, AAV6) or modified capsid variants (AAV2-L1, AAV6.2) have been described previously as highly potent vectors to enable efficient gene delivery to the lung via both, local (intranasal, intratracheal, inhalative) or systemic (intravenous) routes of administration.

FIG. 10 provides examples of AAV-mediated gene delivery to the lung by different AAV serotypes or capsid variants. (A) Immuno-histological staining of green fluorescent protein (GFP) expression in lung sections from C57BL/6J mice 2 weeks after intravenous injection of AAV2-L1-GFP (3×10¹¹ vg/mouse), a recently described AAV2 variant harboring a peptide insertion motive to enable lung-specific gene delivery following systemic administration (Körbelin J et al., 2016). No specific signals beyond background staining were observed in the PBS control group. Representative images from two mice (ms 1, ms 2) out of n=6 animals per group are shown. (B) Assessment of AAV2-L1 bio-distribution by in vivo imaging in FVB/N mice (Published data: Korbelin J et al., 2016). Lung-specific expression of firefly luciferase (fLuc) was observed 2 weeks after intravenous injection of fLuc-expressing AAV2-L1 vector at a dose of 5×10¹⁰ vg/mouse. (C) Ex vivo imaging of is mouse lungs prepared from C57BL/6J mice 2 weeks after intra-tracheal instillation of fLuc-expressing AAV5 vectors (2.9×10¹⁰ vg/mouse) or PBS as a negative control. Quantitative lung transduction was observed in AAV5-fLuc treated animals by detecting light emission resulting from fLuc-positive cells in the luminescence (Lum) channel. Brightfield (BF) images of prepared lungs are shown in the upper panel. Representative images from two mice (ms 1, ms 2) out of n=4 animals per group are shown (D) Analysis of AAV6.2-mediated lung delivery in Balb/c mice three weeks after intratracheal application of GFP-expressing AAV6.2 vectors at a dose of 3×10¹¹ vg/mouse. Micrographs of histological lung sections show direct GFP fluorescence (right) and immuno-histological analysis of GFP expression (left). No specific signals beyond background staining were observed in the PBS control group. Representative images of n=5 animals per group are shown.

FIG. 11 provides examples of different miRNA expression cassettes. A) Vector map of CMV-mir181a-scAAV (Double stranded AAV vector genome for simultaneous expression of a cDNA (eGFP) and a miRNA) and CMV-mir181a-mir181b-mir10a-scAAV (Double stranded AAV vector genome for simultaneous expression of three miRNAs). B) Illustration of different miRNA-designs in the miR-E backbone using mir-181b-5p as an example. The first two examples show mir-181b-5p integrated as fully matured miRNA (23 nt) at the passenger or guide position in the miR-E backbone using perfectly matched complementary strands. The second example illustrates a construct design integrating mir-181b-5p as naturally occurring pre-miRNA into the miR-E backbone. Predicted 2D-structure of mir-181b-5p derived from mirBase (http://mirbase.org/).

FIG. 12 shows knock-down efficiencies of miR181a-5p and miR212-5p in the mir-E backbone on GFP expression construct having the corresponding target sequences in the 3′UTR. HEK-293 cells were transiently transfected with the GFP expression construct in combination with a plasmid encoding one of the miRNAs. GFP fluorescence was measured 72h after transfection. Positive control is an optimal mir-E construct whereas the 3′UTR of the GFP construct is lacking the target sequence for the negative control. The mir181a-5p experiment was performed with a construct on the basis of the miR-E backbone, Guide position according to Seq ID NO: 49 and 47. The experiment for miR212-5p was based on a construct (miR-E backbone, Guide position) according to Seq ID NO:61 and 59, respectively, see also FIG. 25. In the experiment for miR29a-3p a construct according to Seq ID NO: 86 was used, and likewise for the control a construct according to Seq ID NO: 83. Notably, Seq ID NO: 49 and Seq ID NO:61 harbor miRNAs which are 1 nt shorter at the 3′ terminus than the respective references sequences of miRNA 212-5p and miRNA 181a-5p according to Seq ID No. 15 and Seq ID No. 17, respectively.

FIG. 13 shows the basal miRNA expression of human orthologues of the murine candidate miRNAs in normal human lung fibroblasts (NHLFs), measured by using small RNA-sequencing with n=6 replicates. Expression levels are depicted as counts per million (cpm). Arrows mark miRNA candidates of particular interest, which were selected for further functional characterization.

FIG. 14 shows the effect of miRNAs on inflammatory IL6 expression in unstimulated or TGFβ1-stimulated A549 cells. IL-6 is one of the major inflammatory cytokines in different fibrotic diseases, e.g. IPF or systemic sclerosis. The cytokine is, amongst others, produced by activated epithelial cells and could stimulate fibroblasts and immune cells, provoking a pro-fibrotic response/transformation. Thus, TGFβ treated A549 lung epithelial cells are a good surrogate model to mimic that pathophysiological aspect of inflammation in IPF. (A) IL6 expression was assessed by transfection of cells with either miRNA control constructs (Ctrl) or mimetic of the depicted miRNA candidates at 2 nM concentration. 24 hours after transfection cells were stimulated with 5 ng/mL TGFβ1 for another 24 hours. Extracted RNA was then reversely transcribed to cDNA and IL6 gene expression was measured by qPCR. (B) Cells were transfected and stimulated as described in (A) and secreted IL6 protein was detected by ELISA measurements in the cell supernatant. Expression levels are expressed relative to the unstimulated miRNA control construct (Ctrl). Triple=cotransfection of miR-10a-5p, miR-181a-5p and miR-181b-5p. n=3 experiments, mean±SD. *p<0.05, **p<0.01, ***p<0.001 (miRNA candidate vs. Ctrl).

FIG. 15A shows the effect of single miRNAs and their combination on the epithelial-mesenchymal transition (EMT) of normal human bronchial epithelial cells (NHBECs).

EMT is seen as one key initiating factor in the generation of fibrotic lung remodeling. By recurrent epithelial cell damage, there is the chronic secretion of the growth factor TGFβ, leading to a transformation of epithelial cells to mesenchymal (like) cells. These cells lose their epithelial cell function/integrity, leading to a decrease in barrier function, capability of air exchange and start to increase their extra cellular matrix deposition. All three aspects are hallmarks of IPF disease. A marker for functional and integer epithelial cells is the cell marker E-Cadherin. A loss of E-Cadherin is seen as a marker for EMT. An increase in E-cadherin is indicative of the maintenance of epithelial characteristics and therefore considered anti-fibrotic. EMT was assessed by transfection of cells with either miRNA control constructs (Ctrl), mimetic of the depicted miRNA candidates at 2 nM concentration or their combination at 4 nM or 12 nM, as illustrated, followed by stimulation with 5 ng/mL TGFβ1. E-cadherin (a marker of epithelial cells) was immuno-stained 72 h later, quantified by high-content cellular imaging, normalized by the number of detected cells and depicted here as fold change between miRNA candidates and control. n=4 replicates, mean±SD. *p<0.05, **p<0.01 (miRNA candidate vs. Ctrl). SSMD: strictly standardized mean difference; #: |SSMD|>2, ##: |SSMD|>3, ###: |SSMD|>5.

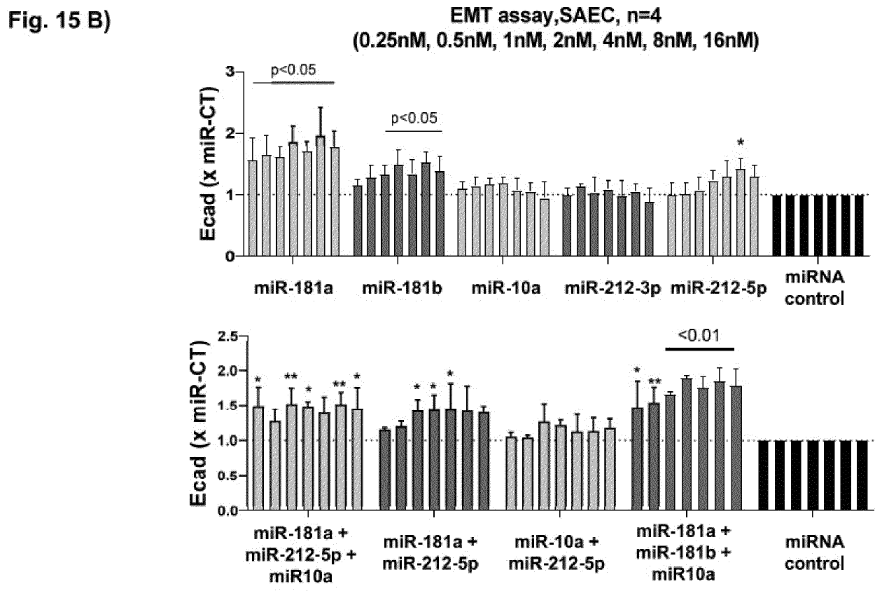

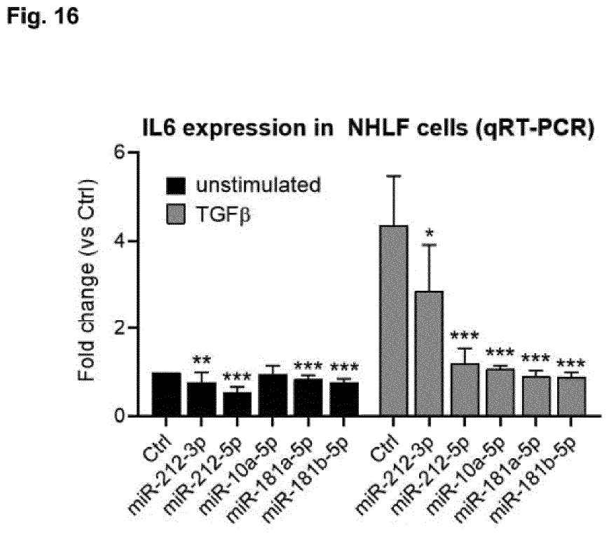

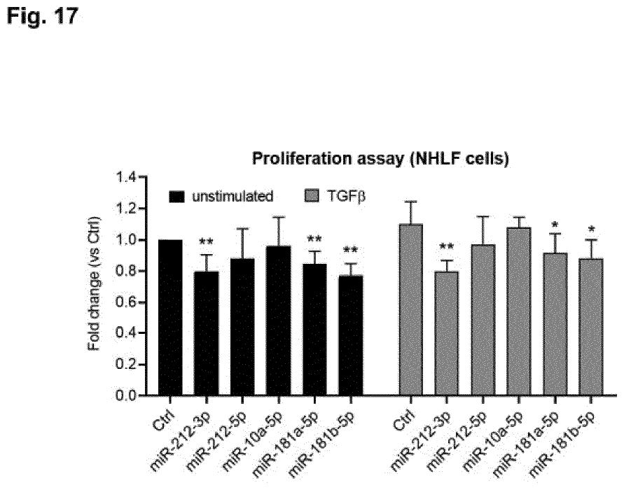

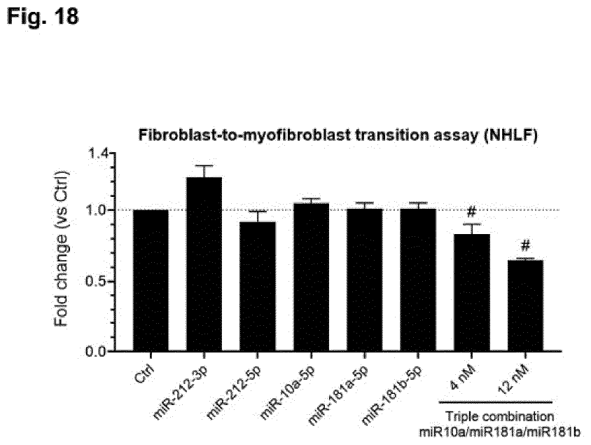

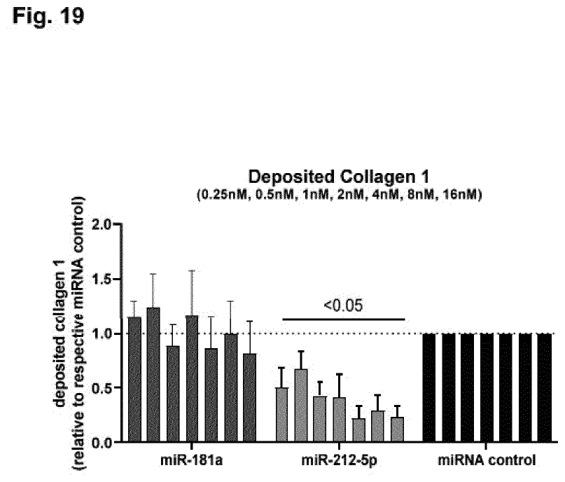

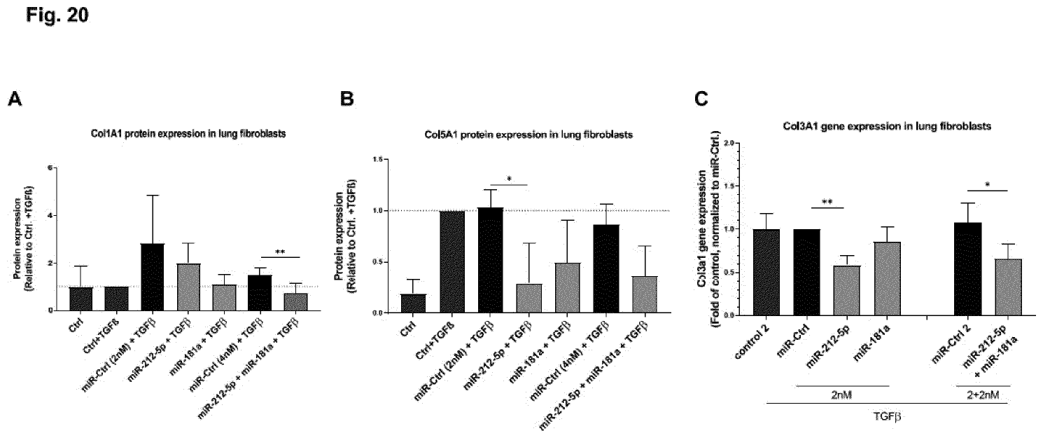

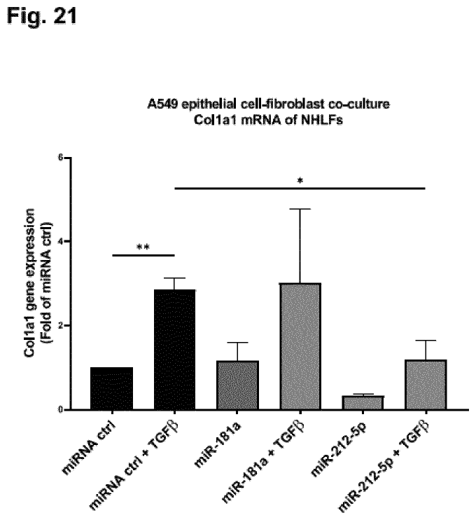

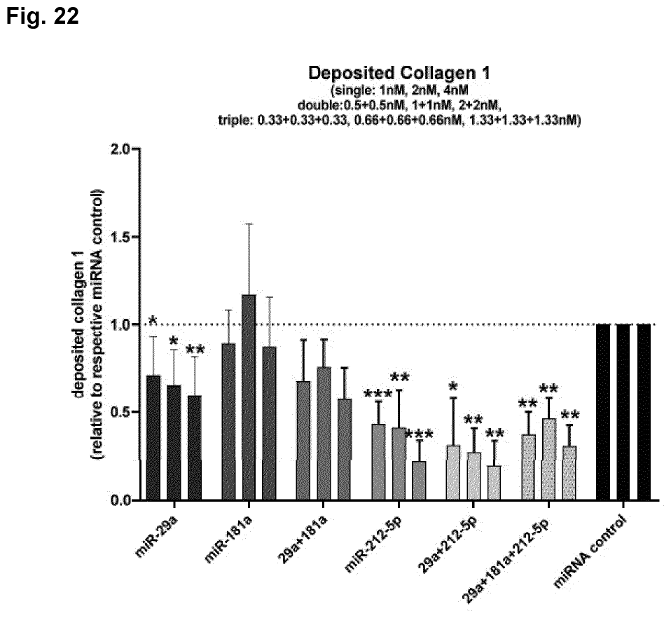

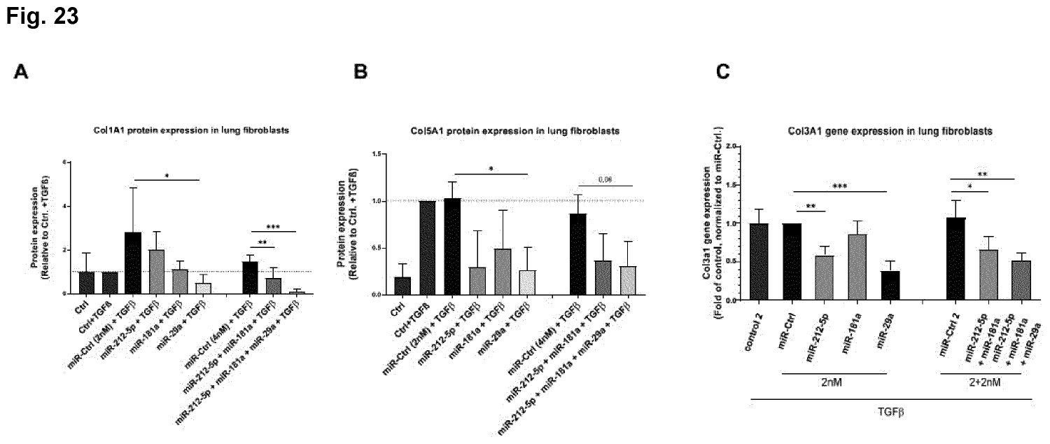

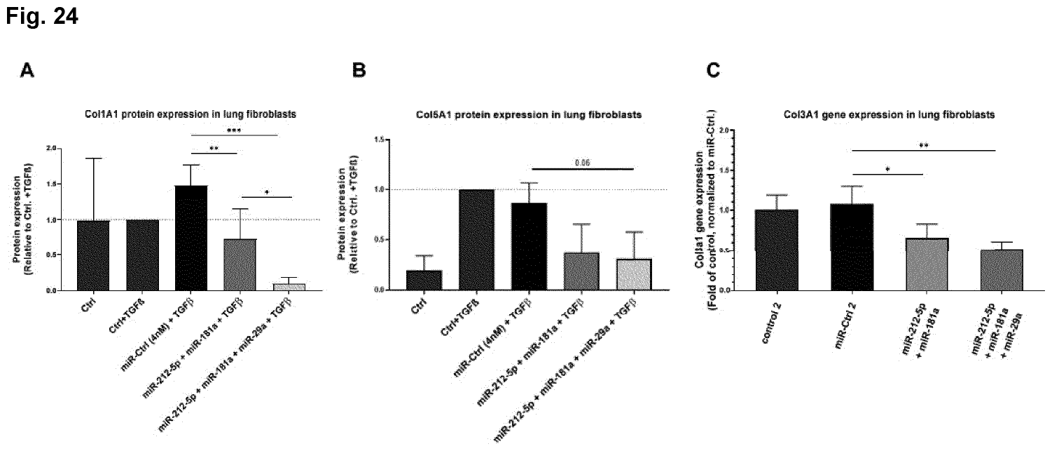

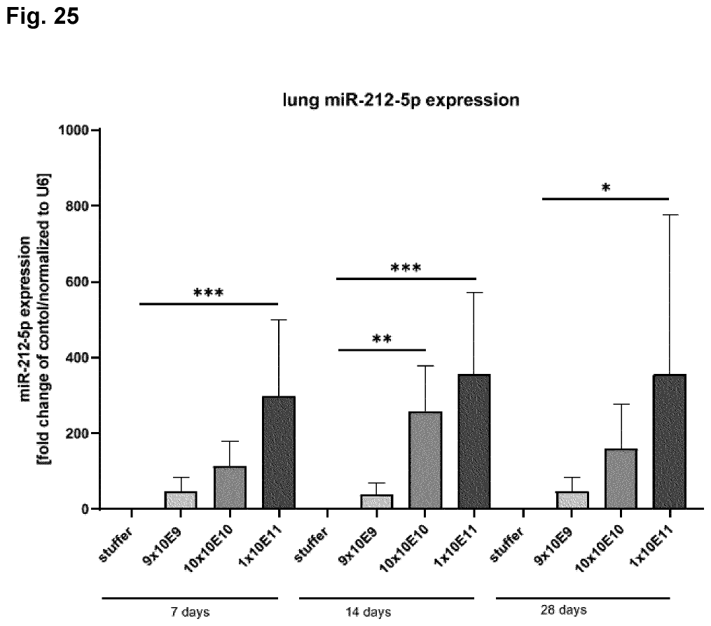

FIG. 15B provides dose/response experiments of single miRNAs (miR181a-5p, miR181b-5p, miR-10a-5p and miR-212-3p and miR-212-5p, respectively) and their combination on the epithelial-mesenchymal transition (EMT) of normal human bronchial epithelial cells (NHBECs). EMT was assessed by transfection of cells with either miRNA control constructs (Ctrl), mimetic of the depicted miRNA candidates at rising concentrations (0.25 nM, 0.5 nM, 1 nM, 2 nM 4 nM, 8 nM, 16 nM). The given concentrations are total concentrations. For double or triple miRNA combinations, the total concentration has to be divided by two or three, respectively, to gain the concentration of involved single miRNA mimetic. Cells were stimulated with 5 ng/mL TGFβ1. E-cadherin (a marker of epithelial cells) was immuno-stained 72 h later, quantified by high-content cellular imaging, normalized by the number of detected cells and depicted here as fold change between miRNA candidates and control. An increase in E-cadherin is indicative of the maintenance of epithelial characteristics and therefore considered anti-fibrotic. n=4 replicates, mean±SD. *p<0.05, **p<0.01 (miRNA candidate vs. Ctrl).

FIG. 16 shows the effect of miRNAs on inflammatory IL6 expression in unstimulated or TGFβ1-stimulated normal human lung fibroblasts (NHLFs)._IL-6 is one of the major inflammatory cytokines in different fibrotic diseases, e.g. IPF or systemic sclerosis. The cytokine is, amongst others, produced by activated epithelial cells and could stimulate fibroblasts and immune cells, provoking a pro-fibrotic response/transformation. But also activated, pro-fibrotic fibroblasts, especially those with a senescent phenotype, producing a lot of inflammatory cytokines, whereas IL-6 is one of the most prominent factors. Thus, TGFβtreated primary human lung fibroblasts (NHLFs) are a good surrogate model to mimic that pathophysiological aspect of inflammation in IPF. IL6 expression was assessed by transfection of cells with either miRNA control constructs (Ctrl) or mimetic of the depicted miRNA candidates at 2 nM concentration. 24 hours after transfection cells were stimulated with 5 ng/mL TGFβ1 for another 24 hours. Extracted RNA was then reversely transcribed to cDNA and IL6 gene expression was measured by qPCR. n=3 replicates, mean±SD. *p<0.05, **p<0.01, ***p<0.001 (miRNA candidate vs. Ctrl).

FIG. 17 shows the effect of miRNAs on the proliferation of unstimulated or TGFβ1-stimulated normal human lung fibroblasts (NHLFs). Controlled fibroblast proliferation is a key aspect of any wound healing process. Fibrotic diseases, including lung fibrosis, are an aberrant wound healing process with aberrant and uncontrolled fibroblast proliferation. Partly this is again driven by the growth factor TGFβ. Thus, determining the proliferation of TGFβ activated lung fibroblast is a key assay to mimic this pathophysiological aspect. A reduction of fibroblast proliferation is seen as an anti-fibrotic effect. Proliferation was assessed by transfection of cells with either miRNA control constructs (Ctrl) or mimetic of the depicted miRNA candidates at 2 nM concentration, followed by stimulation with 5 ng/mL TGFβ1. Proliferation was measured using a spectrophotometric enzymatic WST-1 proliferation assay that measures cellular metabolic activity (mitochondrial dehydrogenase) as a direct correlate of the number of cells. n=3 replicates, mean±SD. *p<0.05, **p<0.01 (miRNA candidate vs. Ctrl).

FIG. 18 shows the effect of single miRNAs and their combination on the fibroblast-to-myofibroblast transition (FMT) of normal human lung fibroblast (NHLFs). FMT is seen as another key initiating factor in the generation of fibrotic lung remodeling. By recurrent epithelial cell damage, there is the chronic secretion of the growth factor TGFβ, leading to an activation of normal, resident lung fibroblasts to myofibroblasts. By the expression of α-smooth muscle actin, myofibroblasts become very contractile and start to increase a massive deposition of many extra cellular matrix components, including collagens. Myofibroblasts are seen as the major driver of the scaring process in fibrotic diseases. Two markers of myofibroblasts are increase cellular levels of a-smooth muscle actin and deposited Collagen, detected via the subunit Col1a1. An increase in E-cadherin is indicative of the maintenance of epithelial characteristics and therefore considered anti-fibrotic. A decrease in collagen is indicative of a loss of myofibroblast characteristics and therefore considered anti-fibrotic. FMT was assessed by transfection of cells with either miRNA control constructs (Ctrl), mimetic of the depicted miRNA candidates at 2 nM concentration or their combination at 4 nM or 12 nM, as illustrated, followed by stimulation with 5 ng/mL TGFβ1. Collagen type 1 α1 (a marker of myofibroblasts), was immuno-stained 72 h later, quantified by high-content cellular imaging, normalized by the number of detected cells and depicted here as fold change between miRNA candidates and control. n=2 donors (4 replicates each), mean±SD. *p<0.05, **p<0.01 (miRNA candidate vs. Ctrl). SSMD: strictly standardized mean difference; #: |SSMD|>2.

FIG. 19 shows the effect of single miRNA-181a-5p and miR-212-5p on collagen 1 deposition of normal and IPF human lung fibroblasts. Collagen 1 deposition was assessed by transfection of cells with either miRNA control constructs (Ctrl), mimetic of the depicted miRNA candidates at rising concentrations (0.25 nM, 0.5 nM, 1 nM, 2 nM 4 nM, 8 nM, 16 nM). Cells were stimulated with 5 ng/ml TGFβ1. Collagen type 1 α1, was immunostained 72 h later, quantified by high-content cellular imaging, normalized by the number of detected cells and depicted here as fold change between miRNA candidates and control. A decrease in collagen is indicative of a loss of myofibroblast characteristics and therefore considered anti-fibrotic. n=7 donors, mean±SD. Two-way ANOVA, Dunnett's multiple comparison.

FIG. 20 shows the effect of miRNA 181a-5p and miR212-5p on the expression of different collagen sub-types in lung fibroblasts. FMT is seen as another key initiating factor in the generation of fibrotic lung remodeling. By recurrent epithelial cell damage, there is the chronic secretion of the growth factor TGFβ, leading to an activation of normal, resident lung fibroblasts to myofibroblasts. Myofibroblasts are seen as the major driver of the scaring process in fibrotic diseases, because they produce many extracellular matrix components, e.g. different types of collagen. Especially Collagen 1, 3 and 5 are seen as components of a fibrotic scar matrix. To detect collagen sub-units (Col1a1, 3a1 and 5a1) in fibroblasts after TGFβ activation is seen as a good surrogate for this pathophysiological aspect in fibrotic diseases. A decrease in collagen subunits is considered as anti-fibrotic. A) Col1a1 and B) Col5a1 protein expression and C) Col3a1 mRNA expression was assessed by transfection of cells with either miRNA control constructs (Ctrl), mimetic of the depicted miRNA candidates at 2 nM (single miRNA) or miRNA combination with 2+2 nM. Cells were stimulated with 5 ng/ml TGFβ1. Collagen type 1α1 and 5α1, was immuno-stained with Western Blot technique, 72 h later and quantified by densitometry. Collagen expression was normalized to GAPDH expression. Col3a1 was quantified 24h later via RT-qPCR. Col 3a1 mRNA expression was normalized with the delta/delta cT method to HPRT mRNA. A decrease in collagens is indicative for fibrosis reduction. Depicted are fold changes between miRNA candidates and control+TGF β1 for A (n=5) and B (n=3) or fold changes between miRNA candidates and miRNA control+TGF β1 for C (n=4). Depicted are means±SD. * p<0.05, ** p<0.01, One-way-ANOVA, Tukey's multiple comparisons test.

FIG. 21 shows the effect of miRNA 181a-5p and miR212-5p on the mRNA expression of Col1a1 on lung fibroblasts in an A549 epithelial-fibroblast co-culture. Col1a1 mRNA expression was assessed by transfection of cells with either miRNA control constructs (Ctrl), mimetic of the depicted miRNA candidates at 2 nM. A549 cells were seeded to 100% confluence on a permeable stimulated cell filter, with sub-cultured lung fibroblasts. A549 cells and fibroblast were separated by the filter, but allowing the flow of A549 secreted factors to the fibroblasts. Only A549 cells were stimulated with 5 ng/ml TGFβ1, whereas sub-seeded lung fibroblasts were not stimulated with exogenous TGFβ1. Collagen type 1α1 mRNA was quantified in lung fibroblasts 24h later via RT-qPCR. Col 1α1 mRNA expression was normalized with the delta/delta cT method to HPRT mRNA. A decrease in collagens is indicative for fibrosis reduction. Depicted are fold changes between miRNA candidates and miRNA control+TGF β1 (n=3). Depicted are means±SD. * p<0.05, ** p<0.01, One-way-ANOVA, Tukey's multiple comparisons test.

FIG. 22 shows the effect of single miRNA-29a-3p, miRNA-181a-5p and miR-212-5p as well as combinations of theses miRNAs on collagen 1 deposition of normal and IPF human lung fibroblasts. FMT is seen as another key initiating factor in the generation of fibrotic lung remodeling. By recurrent epithelial cell damage, there is the chronic secretion of the growth factor TGFβ, leading to an activation of normal, resident lung fibroblasts to myofibroblasts. By the expression of a-smooth muscle actin, myofibroblasts become very contractile and start to increase a massive deposition of many extra cellular matrix components, including collagens. Myofibroblasts are seen as the major driver of the scaring process in fibrotic diseases. Two markers of myofibroblasts are increase cellular levels of α-smooth muscle actin and deposited Collagen, detected via the subunit Col1a1. A decrease in collagen is indicative of a loss of myofibroblast characteristics and therefore considered anti-fibrotic. Collagen 1 deposition was assessed by transfection of cells with either miRNA control constructs (Ctrl), mimetic of the depicted miRNA candidates at rising concentrations (for single miRNAs: 1 nM, 2 nM 4 nM; for dual combinations: 0.5 nM each, 1 nM each, 2 nM each; for triple combination: 0.33 each, 0.66 nM each or 1.33 nM each). Cells were stimulated with 5 ng/ml TGFβ1. Collagen type 1 α1, was immuno-stained 72 h later, quantified by high-content cellular imaging, normalized by the number of detected cells and depicted here as fold change between miRNA candidates and control. A decrease in collagen is indicative of a loss of myofibroblast characteristics and therefore considered anti-fibrotic. For single miRNA experiments n=7 donors/for miRNA combination experiments n=4, mean±SD. Two-way ANOVA, Dunnett's multiple comparison.

FIG. 23 shows the effect of single miRNA-29a-3p, miRNA 181a-5p and miR212-5p as well as combinations of these miRNAs on the expression of different collagen sub-types in lung fibroblasts (healthy and IPF). FMT is seen as another key initiating factor in the generation of fibrotic lung remodeling. By recurrent epithelial cell damage, there is the chronic secretion of the growth factor TGFβ, leading to an activation of normal, resident lung fibroblasts to myofibroblasts. Myofibroblasts are seen as the major driver of the scaring process in fibrotic diseases, because they produce many extracellular matrix components, e.g. different types of collagen. Especially Collagen 1, 3 and 5 are seen as components of a fibrotic scar matrix. To detect collagen sub-units (Col1a1, 3a1 and 5a1) in fibroblasts after TGFβ activation is seen as a good surrogate for this pathophysiological aspect in fibrotic diseases. A decrease in collagen subunits is considered as anti-fibrotic. A) Col1a1 and B) Col5a1 protein expression and C) Col3a1 mRNA expression was assessed by transfection of cells with either miRNA control constructs (Ctrl), mimetic of the depicted miRNA candidates at 2 nM (single miRNA), at 2 nM+2 nM for miRNA combination and 1.3 nM for each miRNA in the triple combination. Cells were stimulated with 5 ng/ml TGFβ1. Collagen type 1α1 and 5a1, was immuno-stained with Western Blot technique, 72 h later and quantified by densitometry. Collagen expression was normalized to GAPDH expression. Col3a1 was quantified 24h later via RT-qPCR. Col 3a1 mRNA expression was normalized is with the delta/delta cT method to HPRT mRNA. A decrease in collagens is indicative for fibrosis reduction. Depicted are fold changes between miRNA candidates and control+TGF β1 for A (n=5) and B (n=3) or fold changes between miRNA candidates and miRNA control+TGF β1 for C (n=4). Depicted are means±SD. * p<0.05, ** p<0.01, One-way-ANOVA, Tukey's multiple comparisons test.

FIG. 24 shows a subset of the results shown in FIG. 23.

FIG. 25 shows miR-212-5p, 22nt lung expression after expression of an AAV-miR-212-5p, 22nt cassette. Mice were intratrachealy instilled with stuffer negative control AAV or three rising dosages (9×10⁹ vg, 10×10¹⁰ vg and 1×10¹¹ vg) of miR-212-5p-AAV (22 nt). Mice were euthanized on day 7, day 14 and day 28 after AAV instillation. Lungs were snap frozen in liquid nitrogen and processed to frozen lung powder for total RNA isolation. Depicted are fold changes of miR-212-5p (22 nt) between different AAV dosages in comparison to stuffer control for each individual time point. Depicted are means±SD. Stuffer group n=6-7, miR-212-5p AAV groups n=7. *p<0.05, **p<0.01, ***p<0.001, One-way-ANOVA, Dunnets multiple comparison test within distinct time points. The experiment was based on a construct according to Seq ID NO: 61 for expressing miR-212-5p, 22nt according to SEQ ID NO:99. For the corresponding plasmid see Seq ID NO: 91.

### SUMMARY OF THE INVENTION

The invention relates to a viral vector comprising: a capsid and a packaged nucleic acid, wherein the packaged nucleic acid codes for two or more miRNAs, wherein the two or more miRNAs comprise the miRNA of Seq ID No. 92 and the miRNA of Seq ID No. 15 or a fragment of the latter having the sequence of Seq ID No. 99. The invention also relates to a viral vector comprising: a capsid and a packaged nucleic acid, wherein the packaged nucleic acid codes for two or more miRNAs, wherein the two or more miRNAs comprise the miRNA of Seq ID No. 92 and the miRNA of Seq ID No. 17 or a fragment of the latter having the sequence of Seq ID No. 100. In a particularly preferred embodiment, the invention relates to a viral vector comprising: a capsid and a packaged nucleic acid, wherein the packaged nucleic acid codes for two or more miRNAs, wherein said miRNAs comprise the miRNA of Seq ID No. 92 and the miRNA of Seq ID No. 15 or a fragment thereof having the sequence of Seq ID No. 99 and the miRNA of Seq ID No. 17 or a fragment thereof having the sequence of Seq ID No. 100. The invention therefore refers to the use of selected miRNAs that have been found effective when being used in combination with each other. The miRNAs include the miRNA of mir-29a-3p (Seq ID no. 92) either in combination with the miRNA of mir-212-5p (Seq ID no. 15) or the miRNA of mir-181a-5p (Seq ID no. 17). In addition, it has been found herein that the miRNA mir-29a-3p can also be combined with fragments of the miRNAs mir-212-5p and mir-181a-5p that lack the terminal nucleotide at the 3′ end of the molecule. These fragments of the miRNAs mir-212-5p and mir181a-5p are set forth herein as Seq ID No. 99 and Seq ID No. 100, respectively. The RNA molecules of Seq ID No. 99 and Seq ID No. 100 are considered as self-contained miRNAs in the context of the present invention. Since the deletion at the 3′-terminus in Seq ID No. 99 and Seq ID No. 100 compared to the authentic mRNA of Seq ID No. 15 and 17, respectively, is remote from the seed region and the region of nucleotides at 13-16 of the miRNA, the specifity of the miRNA according to Seq ID No. 99 and Seq ID No. 100 is acceptable (Grimson et al., 2007). It was shown by Chen, T. et al. that miR-212-5p increase could reduce RVSP and pulmonary vessel wall remodeling in a mouse model of pulmonary hypertension (Chen, T. et al., 2018, Chen, T. et al., 2019). For silicosis context see Jiang, R. et al., 2019 and Yang, X. et al. 2018.

The invention therefore relates to a viral vector comprising: a capsid and a packaged nucleic acid, wherein the nucleic acid augments either (i) the miRNA of Seq ID No. 92 or (ii) miRNA downregulated in a Bleomycin-induced lung fibrosis model or in an AAV-TGFβ1-induced lung fibrosis model, wherein the miRNA comprises miRNA of Seq ID 15 or a fragment thereof having the sequence of Seq ID No. 99 or the miRNA of Seq ID No. 17 or a fragment thereof having the sequence of Seq ID No. 100, or (iii) both (i) and (ii). In one embodiment, the miRNA(s) that are downregulated in a Bleomycin-induced lung fibrosis model or in an AAV-TGFβ1-induced lung fibrosis model and which are augmented by the packaged nucleic acid further comprise the miRNA of Seq ID No. 19. In another embodiment, the one or more miRNAs which are augmented by the packaged nucleic acid comprise the miRNA of Seq ID No. 92 and the miRNA of Seq ID No. 15 or a fragment thereof having the sequence of Seq ID No. 99 and the miRNA of Seq ID No. 19. In another embodiment, the one or more miRNAs which are augmented by the packaged nucleic acid comprise the miRNA of Seq ID No. 92 and the miRNA of Seq ID No. 17 or a fragment thereof having the sequence of Seq ID No. 100 and the miRNA of Seq ID No. 19.

Augmentation in this context means that the level of the respective miRNA in the transduced cell is increased as a result of the transduction of the target cell, which is preferably a lung cell.

The invention further relates to a viral vector comprising: a capsid and a packaged nucleic acid, wherein the nucleic acid augments either (i) the miRNA of Seq ID No. 92 or (ii) miRNA downregulated in a Bleomycin-induced lung fibrosis model or in an AAV-TGFβ1-induced lung fibrosis model, wherein the miRNA comprises the miRNA of Seq ID 15 or a fragment thereof having the sequence of Seq ID No. 99 or the miRNA of Seq ID No. 17 or a fragment thereof having the sequence of Seq ID No. 100, or (iii) both (i) and (ii) and wherein the nucleic acid further inhibits miRNA selected form the group consisting of miRNAs of Seq ID No 1, 2, 3, 4, 5, 6, 7, 8, 9, 10, 11, 12, 13, 14, and 16 or the closest human homolog of respective sequences in case of miRNAs with partial sequence conservation.

Inhibition in this context means that the function of the respective miRNA in the transduced cell is reduced or abolished by complementary binding as a result of the transduction of the target cell.

In one embodiment, the invention relates to a viral vector comprising: a capsid and a packaged nucleic acid that codes for one or more miRNA that are downregulated in a Bleomycin-induced lung fibrosis model or in an AAV-TGFβ1-induced lung fibrosis model:
a) In one preferred embodiment, the one or more miRNA encoded by the packaged nucleic acid comprise the miRNA of Seq ID No. 92. In another embodiment, the one or more miRNAs encoded by the packaged nucleic acid comprise
(i) the miRNA of Seq ID No. 92 and the miRNA of Seq ID No. 15 or a fragment thereof having the sequence of Seq ID No. 99 or
(ii) the miRNA of Seq ID No. 92 and the miRNA of Seq ID No. 17 or a fragment thereof having the sequence of Seq ID No. 100 or
(iii) the miRNA of Seq ID No. 92 and the miRNA of Seq ID No. 15 or a fragment thereof having the sequence of Seq ID No. 99 and the miRNA of Seq ID No. 17 or a fragment thereof having the sequence of Seq ID No. 100, or
(iv) the miRNA of Seq ID No. 92 and the miRNA of Seq ID No. 15 or a fragment thereof having the sequence of Seq ID No. 99 and the miRNA of Seq ID No. 19, or
(v) the miRNA of Seq ID No. 92 and the miRNA of Seq ID No. 17 or a fragment thereof having the sequence of Seq ID No. 100 and the miRNA of Seq ID No. 19.
b) In one embodiment, the one or more miRNA encoded by the packaged nucleic acid comprise
(i) the miRNA of Seq ID No. 92 and the miRNA of Seq ID No. 15 or a fragment thereof having the sequence of Seq ID No. 99 and the miRNA of Seq ID No. 18 or
(ii) the miRNA of Seq ID No. 92 and the miRNA of Seq ID No. 17 or a fragment thereof having the sequence of Seq ID No. 100 and the miRNA of Seq ID No. 18.

It is understood that the nucleic acid usually comprises coding and non-coding regions and that the encoded miRNA up- or downregulated in a Bleomycin-induced lung fibrosis model or in an AAV-TGFβ1-induced lung fibrosis model results from transcription and subsequent maturation steps in target cell transduced by the viral vector.

It is understood that the nucleic acid usually comprises coding and non-coding regions and that the encoded RNA inhibiting the function of one or more miRNA that is upregulated in a Bleomycin-induced lung fibrosis model or in an AAV-TGFβ1-induced lung fibrosis model results from transcription and potentially, but not necessarily, subsequent maturation steps in target cell transduced by the viral vector.

Viral vectors according to the present invention are selected so that they have the potential to transduce lung cells. Non-limiting examples of viral vectors that transduce lung cells include, but are not limited to lentivirus vectors, adenovirus vectors, adeno-associated virus vectors (AAV vectors), and paramyxovirus vectors. Among these, the AAV vectors are particularly preferred, especially those with an AAV-2, AAV-5 or AAV-6.2 serotype. AAV vectors having a recombinant capsid protein comprising Seq ID No. 29, 30 or 31 are particularly preferred (see WO 2015/018860). In one embodiment, the AAV vector is of the AAV-6.2 serotype and comprises a capsid protein of the sequence of Seq ID No. 82.

The sequence coding for the miRNA thereby augmenting its function and the sequence coding for an RNA that inhibits the function of one or more miRNA may or may not be is within the same transgene.

In one embodiment, the invention relates to a viral vector comprising: a capsid and a packaged nucleic acid comprising one or more transgene expression cassettes comprising:
a transgene that codes for two or more miRNAs, said two or more miRNAs comprising the miRNA of Seq ID No. 92 and the miRNA of Seq ID No. 15 or a fragment thereof having the sequence of Seq ID No. 99, or comprising the miRNA of Seq ID No. 92 and the miRNA of Seq ID No. 17 or a fragment thereof having the sequence of Seq ID No. 100,
and a transgene that codes for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

Accordingly, the transgene that codes for a miRNA thereby augmenting its level and the transgene that codes for an RNA that inhibits the function of one or more miRNA are contained in different expression cassettes.

In one embodiment, the invention relates to a viral vector comprising: a capsid and a packaged nucleic acid comprising one or more transgene expression cassettes comprising a transgene that codes
for two or more miRNAs, said two or more miRNAs comprising the miRNA of Seq ID No. 92 and the miRNA of Seq ID No. 15 or a fragment thereof having the sequence of Seq ID No. 99, or comprising the miRNA of Seq ID No. 92 and the miRNA of Seq ID No. 17 or a fragment thereof having the sequence of Seq ID No. 100, and further codes
for an RNA that inhibits the function of one or more miRNAs selected from the group consisting of the miRNAs of Seq ID Nos. 1, 2, 3, 4, 5, 6, 7, 8, 9, 10, 11, 12, 13, 14, 16, 34, 35 and 36.

Accordingly, one transgene codes for both a miRNA thereby augmenting its function and for a RNA that inhibits the function of one or more miRNA.

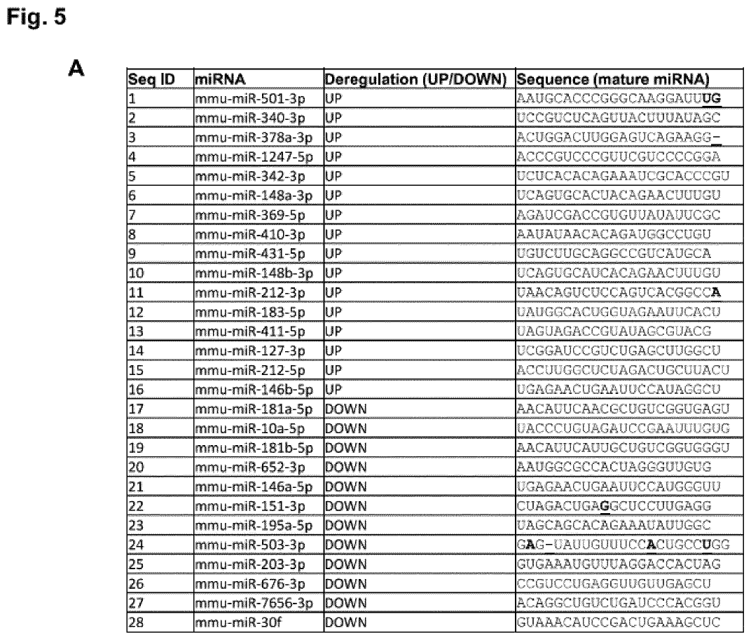

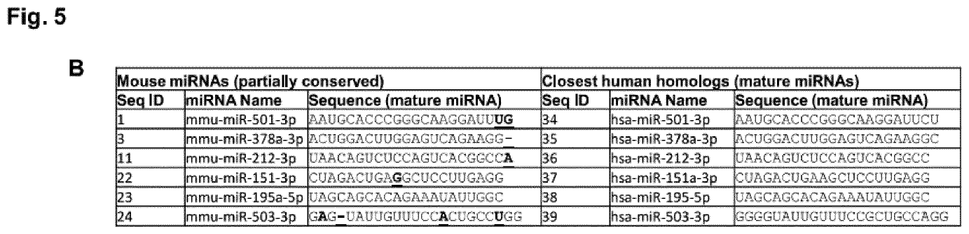

In another embodiment of the invention a viral vector is provided, wherein the miRNA is selected from the group consisting of miRNAs of Seq ID No. 15, 17, 18, 19, 20, 21, 22, 23, 24, 25, 26, 27, 28, 92, 99 and 100 or the closest human homolog of respective sequences in case of miRNAs with partial sequence conservation. In this group, the conserved miRNA, namely 15, 17, 18, 19, 20, 21, 22, 24, 25, 26, 92, 99, 100 or their closest human homolog are most preferred. The closest human homolog of the respective sequences is shown in FIG. 5 B.

In a further embodiment of the invention a viral vector is provided, wherein the nucleic acid has an even number of transgene expression cassettes and optionally the transgene expression cassettes comprising (or consisting of) a promotor, a transgene and a polyadenylation signal, wherein promotors or the polyadenylation signals are positioned opposed to each other.

The viral vector is a recombinant AAV vector in one embodiment of the invention and has either the AAV-2 serotype, AAV-5 serotype or the AAV-6.2 serotype in other embodiments of the invention.

In a different embodiment of the invention a viral vector is provided, wherein the capsid comprises a first protein that comprises the sequence of Seq ID No. 29 or 30 (see WO 2015/018860).
i) In a further embodiment of the invention a viral vector is provided, wherein the capsid comprises a first protein that is 80% identical, more preferably 90%, most preferred 95% to a second protein having the sequence of Seq ID No. 82, whereas one or more gaps in the alignment between the first protein and the second are allowed
ii) In a different embodiment of the invention a viral vector is provided, wherein the capsid comprises a first protein that is 80% identical, more preferably 90%, most preferred 95% identical to a second protein of Seq ID No. 82 whereas a gap in the alignment between the first protein and the second protein is counted as a mismatch.
iii) In a different embodiment of the invention a viral vector is provided, wherein the capsid comprises a first protein that is 80% identical, more preferably 90%, most preferred 95% identical to a second protein of Seq ID No. 82, whereas no gaps in the alignment between the first protein and the second protein are allowed.

For all embodiments (i) to (iii): For the determination of the identity between a first protein and a reference protein, any amino acid that has no identical counterpart in the alignment between the two proteins counts as mismatch (including overhangs with no counterpart). For the determination of identity, the alignment is used which gives the highest identity score.

The packaged nucleic acid may be single or double-stranded. An alternative especially for AAV vectors is to use self-complementary design, in which the vector genome is packaged as a double-stranded nucleic acid. Although the onset of expression is more rapid, the packaging capacity of the vector will be reduced to approximately 2.3 kb, see Naso et al. 2017, with references.

A further aspect of the invention is one of the described viral vectors for use in the treatment of a lung disease, preferably an ILD. The diseases that can be treated according to the present invention are preferably selected from the group consisting of PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, pulmonary hypertension (PH), fibrotic silicosis, systemic sclerosis ILD and sarcoidosis, and fibrosarcoma.

Delivery Strategies for Recombinant AAV Therapeutics are also referred in e.g. Naso et al, 2017.

A double stranded plasmid vector comprising said AAV vector genome is a further embodiment of the invention.

A further embodiment of the invention relates to this miRNA inhibitor for use as a medicinal product.

The present invention also contemplates the use of miRNA mimetics for the prevention and/or treatment of a of a lung disease, preferably an ILD. The lung diseases that can be treated with the miRNA mimetics of the invention are set out above and include fibroproliferative disorder such as ILD, PF-ILD, and IPF. The miRNA mimetics of the present invention typically and preferably consist of a contiguous nucleotide sequence of a total of 21, 22 or 23 contiguous nucleotides in length. The length of the miRNA mimetics (i.e. the length of the “oligomer of nucleotides” in case of a single-strand mimetic or the length of the “oligomer of nucleotides” (i.e. the sense strand) in case of a double-strand mimetic that contains said oligomer besides other oligonucleotides bound to said oligomer) typically and preferably matches the length of the respective miRNA they mimic. In case of miRNA mimetics of a miRNA that has 23 nt, such as miR-181a-5p or miRNA-212-5p, the length of the miRNA mimetics (i.e. the oligomer in case of a single-strand mimetic or the sense strand of the double-strand mimetic) is either 23 nt (preferred) or 22 nt with the proviso that one nucleotide at the 3′-terminus is missing. Since the deletion at the 3-terminus compared to the authentic mRNA (see e.g. Seq ID No. 99 and 100) is remote from the seed region and the region of nucleotides at 13-16 of the miRNA, the specificity of the corresponding miRNA mimetics is acceptable (Grimson et al., 2007).

A further embodiment of the invention therefore is a combination of miRNA mimetics for use in a method of prevention and/or treatment of a fibroproliferative disorder, such as ILD, PF-ILD, or IPF wherein the combination comprises (i) a mimetic of the miRNA having the sequence of Seq ID No. 92, and (ii) a mimetic of the miRNA having the sequence of Seq ID No. 15 and/or a mimetic of the miRNA having the sequence of Seq ID No. 17. The combination of miRNA mimetics may further comprise one or more mimetic of an-miRNA which has a sequence selected from the group consisting of Seq ID No. 18, 19, 20, 21, 22, 23, 24, 25, 26, 27, 37, 38 and 39, preferably selected from the group consisting of Seq ID Nos. 18 and 19. In one embodiment, a miRNA mimetic is provided for use in a method of prevention and/or treatment of a fibroproliferative disorder, such as ILD, PFILD or IPF, wherein miRNA has the sequence of Seq ID No. 92, and wherein the method further comprises the administration of a mimetic of an miRNA that has the sequence of Seq ID No. 15. In another embodiment, a miRNA mimetic is provided for use in a method of prevention and/or treatment of a fibroproliferative disorder, such as ILD, PF-ILD or IPF, wherein miRNA has the sequence of Seq ID No. 92, and wherein the method further comprises the administration of a mimetic of a miRNA that has the sequence of Seq ID No. 17. The prevention and/or treatment preferably further comprises the administration of a mimetic for a miRNA having the sequence of Seq ID No. 18 or of a mimetic for a miRNA having the sequence of Seq ID No. 19.

Likewise, in a further embodiment a miRNA mimetic is provided for use in a method of prevention and/or treatment of a fibroproliferative disorder, such as ILD, PF-ILD or IPF, wherein the miRNA has the Seq ID No. 92. The prevention and/or treatment further comprises the administration of a mimetic for a miRNA having the sequence of Seq ID No. 15 or of a mimetic for a miRNA having the sequence of Seq ID No. 17. Even more preferably,
the prevention and/or treatment comprises the administration of a mimetic for a miRNA having the sequence of Seq ID No. 92, a mimetic for a miRNA having the sequence of Seq ID No. 15 and of a mimetic for a miRNA having the sequence of Seq ID No. 18, or
the prevention and/or treatment comprises the administration of a mimetic for a miRNA having the sequence of Seq ID No. 92, a mimetic for a miRNA having the sequence of Seq ID No. 17 and of a mimetic for a miRNA having the sequence of Seq ID No. 18, or
the prevention and/or treatment comprises the administration of a mimetic for a miRNA having the sequence of Seq ID No. 92, a mimetic for a miRNA having the sequence of Seq ID No. 15 and of a mimetic for a miRNA having the sequence of Seq ID No. 17.

A further embodiment of the invention is (i) a miRNA mimetic of a miRNA having the sequence of Seq ID No. 92 and (ii) a miRNA mimetic of a miRNA having the sequence of Seq ID No. 15 or a miRNA mimetic of a miRNA having the sequence of Seq ID No. 17, for the treatment of a fibroproliferative disorder such as ILD, PF-ILD or IPF and a pharmaceutical composition comprising these miRNA mimetics and a pharmaceutical-acceptable carrier or diluent.

A further embodiment of the invention is a pharmaceutical composition comprising a miRNA mimetic of a miRNA having the sequence of Seq ID No. 92 and a miRNA mimetic of a miRNA having the sequence of Seq ID No. 15, and a pharmaceutical-acceptable carrier or diluent. Another embodiment of the invention is a pharmaceutical composition comprising a miRNA mimetic of a miRNA having the sequence of Seq ID No. 92 and a miRNA mimetic of a miRNA having the sequence of Seq ID No. 17, and a pharmaceutical-acceptable carrier or diluent. Preferably, the miRNA mimetics in the composition are packed in lipid nanoparticles (LNPs). The LNPs may preferably have a mean particle size of the LNPs is between 30 and 200 nm. The pharmaceutical composition may further comprise to 65 mol % of ionizable lipids.

In any of the above embodiments, the mimetic of the miRNA having the sequence of Seq ID No. 92 preferably is (in case of a single-single stranded mimetic) or contains (in case of a double-stranded mimetic) an oligomer that has the sequence of Seq ID No. 92. Similarly, the mimetic of the miRNA having the sequence of Seq ID No. 15 preferably is or contains an oligomer that has the sequence of Seq ID No. 15 or an oligomer that has the sequence of Seq ID No. 99. The mimetic of the miRNA having the sequence of Seq ID No. 17 preferably is or contains an oligomer that has the sequence of Seq ID No. 17 or an oligomer that has the sequence of Seq ID No. 100.

The invention also provides an miRNA mimetic of miRNA m29a-3p for use in the treatment of a fibroproliferative disorder, such as ILD, PF-ILD or IPF, wherein the miRNA mimetic is (less preferred) or contains (preferred) an oligomer of nucleotides that consists of the sequence selected form the group consisting of Seq ID No. 92, with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer optionally comprises nucleotide analogues that show the base-pairing behavior at the corresponding position (AU and GC) as determined by the sequence of the respective miRNA;
the oligomer is optionally lipid conjugated to facilitate drug delivery,

wherein said prevention and/or treatment further comprises the administration of a mimetic of a miRNA having the sequence of Seq ID No. 15 and/or a mimetic of a miRNA having the sequence of Seq ID No. 17.

In one embodiment, the prevention and/or treatment further comprises the administration of a mimetic of a miRNA having the sequence of Seq ID No. 15. Preferably, the mimetic of the miRNA having the sequence of Seq ID No. 15 is or contains an oligomer of nucleotides that consists of the sequence of Seq ID No. 15 (preferred) or Seq ID No. 99 (less preferred) with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer is optionally lipid conjugated to facilitate drug delivery.

In another embodiment, the prevention and/or treatment further comprises the administration of a mimetic of a miRNA having the sequence of Seq ID No. 17. Preferably, the mimetic of the miRNA having the sequence of Seq ID No. 17 is (less preferred) or contains (preferred) an oligomer of nucleotides that consists of the sequence of Seq ID No. 17 (preferred) or Seq ID No. 100 (less preferred) with the following proviso:
the oligomer optionally comprises nucleotide analogues that show the base-pairing behavior at the corresponding position (AU and GC) as determined by the sequence of the respective miRNA;
the oligomer is optionally lipid conjugated to facilitate drug delivery.

In case, the miRNA mimetics are not delivered being packed in lipid based nano particles (LNPs), it is preferred that the oligomer mentioned in the proviso is lipid conjugated to facilitate drug delivery.

In yet another embodiment, the prevention and/or treatment further comprises the administration of a mimetic of a miRNA having the sequence of Seq ID No. 19. Preferably, the mimetic of the miRNA having the sequence of Seq ID No. 19 is (less preferred) or contains (preferred) an oligomer of nucleotides that consists of the sequence of Seq ID No. 19 with the following proviso:
the oligomer optionally comprises nucleotides with chemical modifications leading to non-naturally occurring nucleotides that show the base-pairing behavior at the corresponding position (AU and GC) as determined by the sequence of the respective miRNA;
the oligomer optionally comprises nucleotide analogues that show the base-pairing is behavior at the corresponding position (AU and GC) as determined by the sequence of the respective miRNA.

Further embodiments of the invention are miRNA mimetics of miRNA 29a-3p (Seq ID No. 92), in combination with mimetics of the miRNA 212-5p (Seq ID No. 15) or miRNA 181a5p (Seq ID No. 17) for use in the treatment of a fibroproliferative disorder, such as ILD, PF-ILD or IPF, and wherein the miRNA mimetics are oligomers of nucleotides that consist of the sequence of Seq ID No. 92, Seq ID No. 15 or 99, and Seq ID No. 17 or 100, respectively, with the following proviso:
the oligomer optionally comprises nucleotides with chemical modifications leading to non-naturally occurring nucleotides that show the base-pairing behavior at the corresponding position (AU and GC) as determined by the sequence of the respective miRNA.

Further embodiments of the invention are miRNA mimetics of miRNA 29a-3p (Seq ID No. 92) in combination with miRNA 212-5p (Seq ID No. 15 or 99) or miRNA 181a-5p (Seq ID No. 17 or 100) for use in the treatment of a fibroproliferative disorder, such as ILD, PFILD or IPF, and wherein the miRNA mimetic is or contains an oligomer of nucleotides that consists of the sequence of Seq ID No. 92, Seq ID No. 15 or 99, and Seq ID No. 17 or 100.

These embodiments are preferred in case, the miRNA mimetics are delivered being packed in lipid based nanoparticles (LNPs). If LNP particles are used for delivery, the dose might be between 0.01 and 5 mg/kg of the mass of miRNA mimetics per kg of subject to be treated, preferably 0.03 and 3 mg/kg, more preferably 0.1 and 0.4 mg/kg, most preferably 0.3 mg/kg. The administration is of the LNP particles preferably systemic, more preferably intravenous.

In case of a double-strand miRNA mimetic, the miRNA mimetic contains an oligomer of nucleotides (sense strand) that is bound to one or more oligonucleotides that are fully or partially complimentary to the sense strand of said miRNA mimetic, said sense strand of miRNA mimetic may or may not form with these one or more oligonucleotides overhang(s) with single stranded regions.

Double-strand miRNA mimetics are preferred.

A further embodiment of the invention relates to a pharmaceutical composition as defined herein above wherein the composition is an inhalation composition.

A further embodiment of the invention relates to a pharmaceutical composition as defined herein above wherein the composition is intended for systemic, preferably intravenous administration.

A further embodiment of the invention is a method of treating or preventing of a fibroproliferative disorder, such as ILD, PF-ILD or IPF, in a subject in need thereof comprising administering to the subject a pharmaceutical composition as defined above.

For example, the use of a miRNA inhibitor or a miRNA mimetic can be effected by the aerosol route for inhibiting fibrogenesis in the pathological respiratory epithelium in subjects suffering from pulmonary fibrosis and thus restoring the integrity of the pathological tissue so as to restore full functionality.

The viral vector is preferably administered as in an amount corresponding to a dose of virus in the range of 1.0×10¹⁰ to 1.0×10¹⁴ vg/kg (virus genomes per kg body weight), although a range of 1.0×10¹¹ to 1.0×10¹² vg/kg is more preferred, and a range of 5.0×10¹¹ to 5.0×10¹² vg/kg is still more preferred, and a range of 1.0×10¹² to 5.0×10¹¹ is still more preferred. A virus dose of approximately 2.5×10¹² vg/kg is most preferred. The amount of the viral vector to be administered, such as the AAV vector according to the invention, for example, can be adjusted according to the strength of the expression of one or more transgenes.

A further aspect of the invention is the use of viral vectors, miRNA inhibitors and miRNA mimetics according to the invention for combined therapy with either Nintedanib or Pirfenidone.

### Used Terms and Definitions

An expression cassette comprises a transgene and usually a promotor and a polyadenylation signal. The promotor is operably linked to the transgene. A suitable promoter may be selectively or constitutively active in a lung cell, such as an epithelial alveolar cell. Specific non-limiting examples of suitable promoters include constitutively active promoters such as the cytomegalovirus immediate early gene promoter, the Rous sarcoma virus long terminal repeat promoter, the human elongation factor 1a promoter, and the human ubiquitin c promoter. Specific non-limiting examples of lung-specific promoters include the surfactant protein C gene promoter, the surfactant protein B gene promoter, and the Clara cell kD (“CC 10”) promoter.

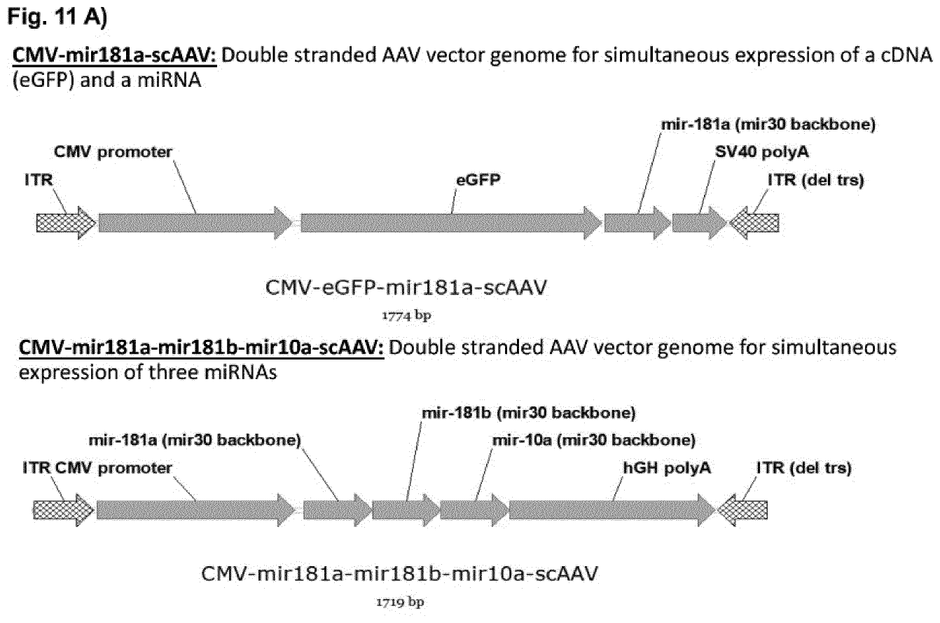

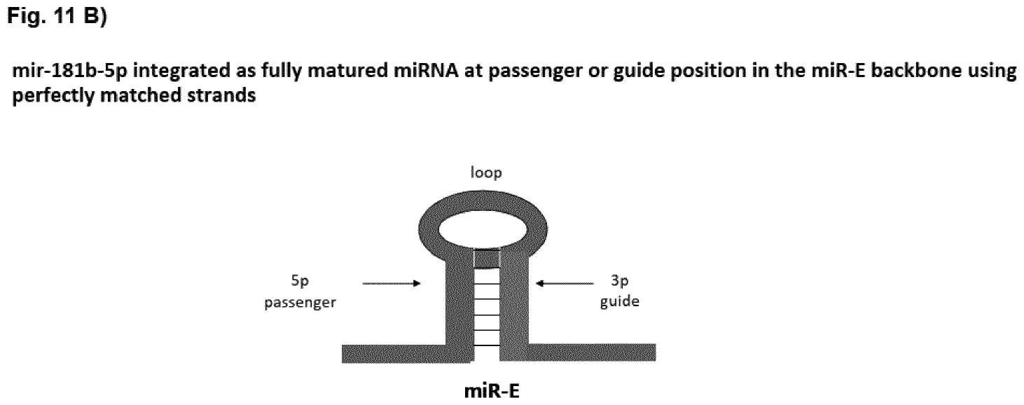

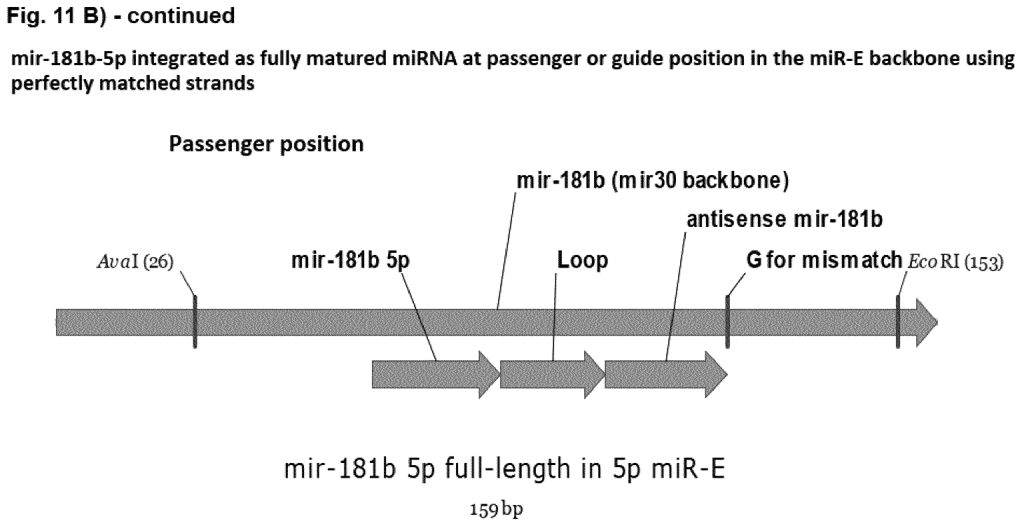

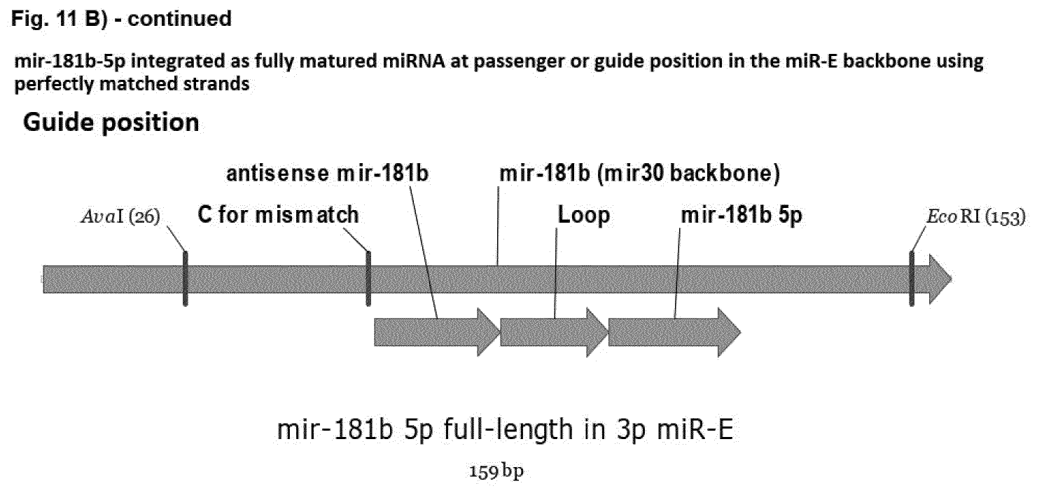

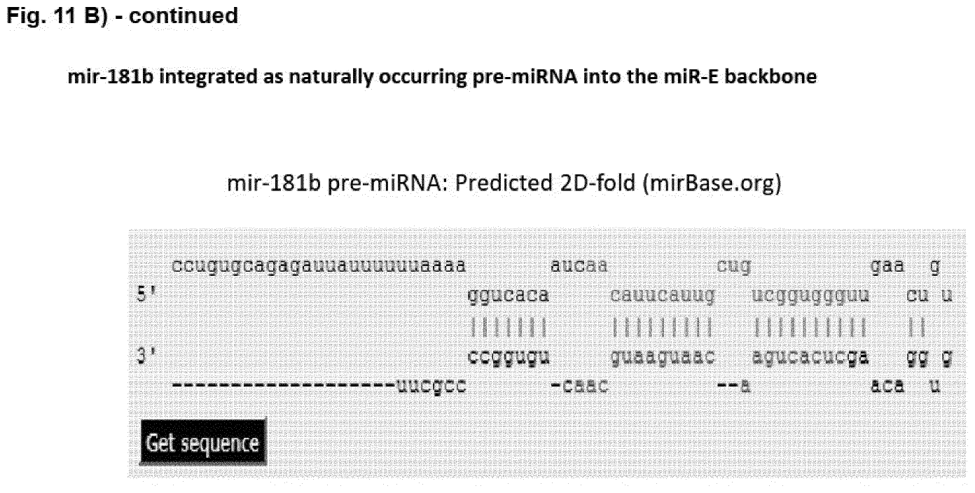

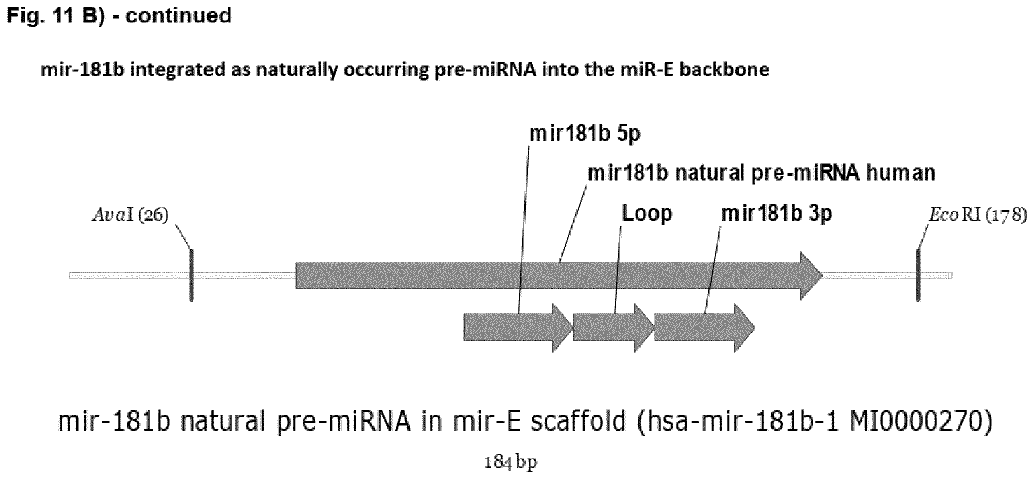

A transgene, depending on the embodiment of the invention, either codes for (i) one or more miRNA e.g. a miRNA having the sequence of Seq ID No. 92 or one or more miRNA that are downregulated in a Bleomycin-induced lung fibrosis model or in an AAV-TGFβ1-induced lung fibrosis model, or (ii) for an RNA that inhibits the function of one or more miRNA that is upregulated in a Bleomycin-induced lung fibrosis model and in an AAV-TGFβ1-induced lung fibrosis model, or for both alternatives (i) and (ii). The transgene may also contain an open reading frame that encodes for a protein for transduction reporting (such as eGFP, see FIG. 11) or therapeutic purposes.

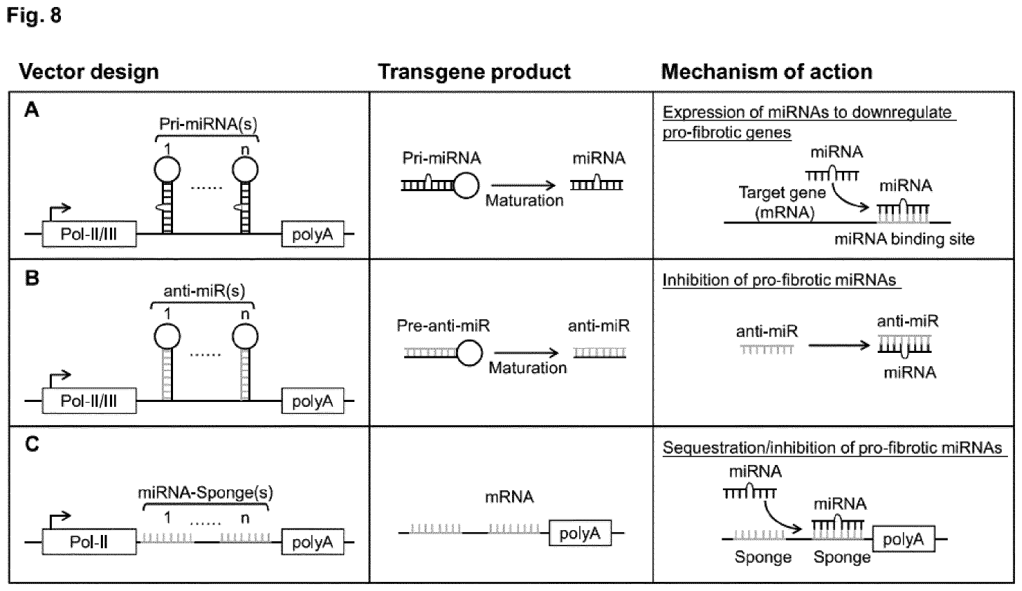

An RNA that inhibits the function of one or more miRNA reduces or abolishes the function of its target miRNA by complementary binding. Two different vector design strategies can be applied, as described in FIGS. 8 B and C:
1). Expression of antisense-like molecules designed to specifically bind to profibrotic miRNAs and thereby inhibit their function (FIG. 8B). Respective molecules, so s called anti-miRs, can be incorporated into expression vectors as short hairpin RNAs (shRNAs) or as artificial miRNAs. In analogy to the miRNA supplementation approach, several miRNA-targeting sequences may be combined in a single vector, thereby enabling inhibition of various target miRNAs.
2) Expression of mRNAs containing several copies of miRNA binding sites, so called sponges, aiming to selectively sequester pro-fibrotic miRNAs and thereby inhibit their function (FIG. 8C). For this alternative the inhibiting RNA is not subject to RNAi processing or RNAi maturation.

The term miRNA inhibitor according to the present invention refers to oligomers consisting of a contiguous sequence of 7 to at least 22 nucleotides in length.

The term nucleotide, as used herein, refers to a glycoside comprising a sugar moiety (usually ribose or desoxyribose), a base moiety and a covalently linked group (linkage group), such as a phosphate or phosphorothioate intemucleotide linkage group. It covers both naturally occurring nucleotides and non-naturally occurring nucleotides comprising modified sugar and/or base moieties, which are also referred to as nucleotide analogues herein. Non-naturally occurring nucleotides include nucleotides which have sugar moieties, such as bicyclic nucleotides or 2′ modified nucleotides or 2′ modified nucleotides such as 2′ substituted nucleotides.

Nucleotides with chemical modifications leading to non-naturally occurring nucleotides comprise the following modifications:
(i) Nucleotides which have non-natural sugar moieties,

Examples are bicyclic nucleotides or 2′ modified nucleotides or 2′ modified nucleotides such as 2′ substituted nucleotides.
(ii) Nucleotides with Phosphorothioate (PS) and Phosphodithioate (PS2) modifications

To improve serum stability and increase blood concentrations as well as improve nuclease resistance of the miRNAs, a sulfur in one or more nucleotides of the miRNA inhibitor or mimic could exchange an oxygen of the nucleotide phosphate group, which is defined as a phosphorothioate (PS). For some sequences, this could be combined or complemented by a second introduction of a sulfur group to an existing PS, which is defined as a Phosphodithioate PS2. PS2 modifications on distinct positions of the sense strand, like on nucleotide 19+20 or 3+12 (counting from the 5′ end), could further increase serum stability and therefore the pharmacokinetic characteristics of the miRNA inhibitor/miRNA mimetic (ACS Chem. Biol. 2012, 7, 1214-1220).
(iii) Nucleotides with Boranophosphat modifications

For some miRNA oligonucleotides, it could be beneficial to exchange one oxygen of the ribose phosphate group against a BH3 group. Boranophosphat modifications on one or more nucleotides could increase serum stability, in case the seed region of miRNA oligonucleotides are not modified by other chemical modifications. Boranophosphat modifications could also increase serum stability of miRNA oligonucleotides (Nucleic Acids Research, Vol. 32 No. 20, 5991-6000).
(iv) Nucleotides with 2′O-methyl modification

Besides or in addition to phosphate modifications, methylation of the oxygen, bound to the carbon C2 in the ribose ring, could be further options for oligonucleotide modifications. 2′O-methyl ribose modification of the sense strand could increase thermal stability and the resistance to enzymatic digestions.
(v) Nucleotides with 2′OH with fluorine modification

It may could also beneficial to modify miRNA oligonucleotides with 2′ OH fluorine modification to enhance serum stability of the oligonucleotide and improve the binding affinity of the miRNA oligonucleotide to its target. 2′ OH fluorine modification, exchanges the hydroxyl group of the carbon C2 in the ribose ring against a fluorine atom. Fluorine modifications could be applied on both strands, sense and anti-sense.

“Nucleotide analogues” are variants of natural oligonucleotides by virtue of modifications in the sugar and/or base moieties. Preferably, without being limited by this explanation, the analogues will have a functional effect on the way in which the oligomer works to bind to its target; for example by producing increased binding affinity to the target and/or increased resistance to nucleases and/or increased ease of transport into the cell. Specific examples of nucleoside analogues are described by Freier and Altman (Nucl. Acid Res, 25: 4429-4443, 1997) and Uhlmann (Curr. Opinion in Drug Development, 3: 293-213, 2000). Incorporation of affinity-enhancing analogues in the oligomer, including Locked Nucleic Acid (LNA™), can allow the size of the specifically binding oligomer to be reduced and may also reduce the upper limit to the size of the oligomer before non-specific or aberrant binding takes place. The term “LNA™” refers to a bicyclic nucleoside analogue, known as “Locked Nucleic Acid” (Rajwanshi et al., Angew Chem. Int. Ed. Engl., 39(9): 1656-1659, 2000). It may refer to an LNA™ monomer, or, when used in the context of an “LNA™ oligonucleotide” to an oligonucleotide containing one or more such bicylic analogues.

Preferably, a miRNA inhibitor of the invention refers to antisense oligonucleotides with sequence complementary to Certain upregulated miRNA (miRNAs selected from the group consisting of the miRNAs of Seq ID Nos. 1, 2, 3, 4, 5, 6, 7, 8, 9, 10, 11, 12, 13, 14, 16, 34, and 36). These oligomers may comprise or consist of a contiguous nucleotide sequence of a total of 7 to at least 22 contiguous nucleotides in length, up to 70% nucleotide analogues (LNA™). The shortest oligomer (7 nucleotides) will likely correspond to an antisense oligonucleotide with perfect sequence complementarity matching to the first 7 nucleotides located at the 5′ end of mature to Certain up regulated miRNA, and comprising the 7 nucleotide sequence at position 2-8 from 5′ end called the “seed” sequence) involved in miRNA target specificity (Lewis et al., Cell. 2005 Jan. 14; 120(1):15-20).

A Certain upregulated miRNA Target Site Blocker refers to antisense oligonucleotides with sequence complementary to Certain upregulated miRNA binding site located on a specific mRNA. These oligomers may be designed according to the teaching of US 20090137504. These oligomers may comprise or consist of a contiguous nucleotide sequence of a total of 8 to 23 contiguous nucleotides in length. These sequences may span from 20 nucleotides in the 5′ or the 3′ direction from the sequence corresponding to the reverse complement of Certain upregulated miRNA “seed” sequence.

The term miRNA mimetic of the invention is a single-stranded or double-stranded oligomer of nucleotides capable of specifically increasing the activity of certain miRNA wherein the term certain miRNA means a miRNA that has a sequence selected from the group consisting of Seq ID No. 15, 17, 18, 19, 20, 21, 22, 23, 24, 25, 26, 27, 37, 38, 39, and 92 preferably of Seq ID No. 92, 15, 17, 19, 18, and 20, most preferred 15, 17 and 19, even more preferred Seq ID No. 15. The term miRNA mimetic encompasses salts, including pharmaceutical acceptable salts. The miRNA mimetic of a miRNA elevates the concentration of functional equivalents of said miRNA in the cell thereby increasing the overall activity of said miRNA.

These miRNA mimetics of the present invention typically and preferably consist of a contiguous nucleotide sequence of a total of 21, 22 or 23 contiguous nucleotides in length. The length of the miRNA mimetics (i.e. the oligonucleotide in case of a single-strand mimetic or the sense strand in case of a double strand mimetic) typically matches the length of the respective miRNA they mimic (preferred).

In case of miRNA mimetics of a miRNA that has 23 nt, such as miR-181a-5p or miRNA-212-5p, the length of the miRNA mimetics (i.e. the oligonucleotide in case of a single strand or the sense strand of the double strand mimetic) the is either 23 nt (preferred) or 22 nt with the proviso that one nucleotide at the 3′-terminus is missing. Since the deletion at the 3′-terminus compared to the authentic mRNA (see e.g. Seq ID NO. 100, 99) is remote from the seed region and the region of nucleotides at 13-16 of the miRNA, the specificity of the corresponding miRNA mimetics is acceptable (Grimson et al., 2007).

miRNA mimetics of miRNA 29a-3p, 212-5p, miRNA 181a-5p, miRNA 181b-5p, and miRNA 10a-5p, respectively are intended for use in the treatment of a fibroproliferative disorder, such as ILD, PF-ILD or IPF, and wherein the miRNA mimetic is or contains an oligomer of nucleotides that consists of the sequence of Seq ID No. 92, of Seq ID No. 15 or 99, of Seq ID No. 17 or 100, Seq ID No. 18, and Seq ID No. 19, respectively with proviso (a), (b) and (c), (a) and (c), (a) and (d), or (c) and (d),
(a) the oligomer optionally comprises nucleotides with chemical modifications leading to non-naturally occurring nucleotides that show the base-pairing behavior at the corresponding position (AU and GC) as determined by the sequence of the respective miRNA, preferably chemical modifications as set forth under (i) to (v) herein above;
(b) the oligomer optionally comprises nucleotide analogues that show the base-pairing behavior at the corresponding position (AU and GC) as determined by the sequence of the respective miRNA; preferably the nucleotide analogues described by Freier and Altman (Nucl. Acid Res., 25: 4429-4443, 1997) and Uhlmann (Curr. Opinion in Drug Development, 3: 293-213, 2000) or bicyclic analogues described herein above;
(c) the oligomer is optionally lipid conjugated to facilitate drug delivery.

Lipid conjugated oligomers are well known in the art, see Osborne et al. NUCLEIC ACID THERAPEUTICS Volume 28, Number 3, 2018 with references.

Oligomer consisting of the sequence of Seq ID No. x means that the oligomer comprises the sequence of Seq ID No. x and has as many covalently attached nucleotide building blocks (optionally with chemical modifications) or nucleotide analogues as indicated in the Seq ID No. x.

The miRNA mimetic may be a single-strand miRNA mimetic or a double-strand miRNA mimetic. A single-stand mimetic is an oligonucleotide with no other oligonucleotide molecule bound thereto with full or partial base-pairing. Double-strand miRNA mimetics are defined as miRNA mimetics that are bound to one or more oligonucleotides that are fully or partially complimentary to the miRNA mimetic and that may or may not form with these oligonucleotides overhangs with single stranded regions. The triple RNA strand design referred to under Example 1.11 is an example for double-stranded miRNA mimetics. A further example is disclosed in Vinnikov et al. (2014), p.10661, 1^{st} col, last paragraph. It is preferred that the miRNA mimetic has at least 80%, more preferably at least 90%, even more preferably more than 95% of the biologic effect of the same amount of the natural miRNA as determined by one or more experiments as described under Example 1.11.

miRNA mimetics or miRNA inhibitors can also be delivered as naturally- and non-naturally occurring nucleotides, packed in lipid nano particles (LNPs). For RNA as cargo molecules, the most effective LNPs contain ionizable lipids with pKa values typically below pH 7 and are composed of up to four components, i.e. ionizable lipids, structural lipids, cholesterol, and polyethyleneglycol (PEG) lipids.

Ionizable lipids include but are not limited to 1,2-dilinoleoyl-3-dimethylamine (DLinDMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLinMC3-DMA) (Naseri N, Valizadeh H, Zakeri-Milani P. Solid lipid nanoparticles and nanostructured lipid carriers: structure, preparation, andapplication. Adv Pharm Bull. 2015; 5(3):305-313), ATX-lipids (Ramaswamy S, Tonnu N, Tachikawa K, et al. Systemic delivery of factor IX messenger RNA for protein replacement therapy. Proc Natl Acad Sci USA. 2017; 114(10): E1941-50), or YSK12-C4-lipids (Sato Y, Hashiba K, Sasaki K, et al. Understanding structure-activity relationships of pH-sensitive cationic lipids facilitates the rational identification of promising lipid nanoparticles for delivering siRNAs in vivo. J Control Release. 2019; 295:140-152.)

Structural lipids include but are not limited to dioleoyl-sn-glycero-3-phosphatidylcholine (DOPC), dimyristoyl-sn-glycero-3-phosphatidylcholine (DMPC), distearoyl-sn-glycero-3-phosphatidylocholine (DSPC), dipalmitoyl-sn-glycero-3-phosphocholine (DPPC), dipalmitoyl-sn-glycero-3-phosphatidylethanolamine (DPPE), dioleoyl-sn-glycero-3-phosphatidylethanolamine (DOPE), 1-palmitoyl-2-oleoyl-sn-glycero-3-phosphatidylcholine (POPC), hydrogenated soybean phosphatidylcholine (HSPC), etc. Cholesterol includes but is not limited to cholesterol and 3-(N—(N0,N0-dimethylaminoethane)carbamoyl) cholesterol, sterols, steroids, etc.

PEG-lipids include but are not limited to 1,2-distearoyl-sn-glycero-3-phosphoethanolamine-N-[amino(polyethylene glycol)-2000 (DSPE-mPEG₂₀₀₀), 1,2-dimyristoyl-snglycero-3-phosphoethanolamine-N-[amino(polyethylene glycol)-2000 (DMPE-mPEG₂₀₀₀), 1,2-dipalmitoyl-sn-glycero-3-phosphoethanolamine-N-[amino(polyethylene glycol)-2000 (DPPE-mPEG₂₀₀₀), and 1,2-dioleoyl-sn-glycero-3-phosphoethanolamine-N-[amino(polyethylene glycol)-2000 (DOPE-mPEG₂₀₀₀) and variations of those PEG-lipids with respect to the PEG length, e.g. PEG₅₀₀, PEG₁₀₀₀, PEG₅₀₀₀, etc.

The LNP formulations can contain distinct proportions of the single LNP components, distinct particle size, and a distinct ratio of positively-chargeable polymer amine (N=nitrogen) groups to negatively-charged nucleic acid phosphate (P) groups (N/P ratio). The preferred formulations comprise or contain 25 to 65 mol % of ionizable lipids, preferably 40 mol %, 5 to 30 mol % of structural lipids, preferably 15 mol %, 15 to 50 mol % cholesterol, preferably 40 mol %, and 1 to 5 mol % of PEG-lipids, preferably 2 mol %. The mean particle size of LNPs can vary between 30 and 200 nm and N/P ratios can vary between 2 to 4, whereas the most preferred nanoparticle size is 100 nm with a N/P ratio of 3.

The most preferred LNP formulation will have the following composition: 40 mol % ionizable lipid consisting of DLinMC3-DMA or ATX lipids, or YSK12-C4-lipids, 15 mol % DSPC, 40 mol % cholesterol, 2 mol % DSPE-mPEG₂₀₀₀ with a particle size of 100 nm and a N/P ratio of 3.

The most preferred miRNA modality for LNP delivery of miRNA mimetics is a double-strand miRNA mimetic, consisting of a complementary passenger sense strand to an antisense strand. The passenger strand will protect the anti-sense strand from endonucleases. Like described by Vinnikov et al., both strands have LNA modified overhangs on the 3′ site, consisting of two nucleotides with LNA modification (Vinnikov et al, 2014). LNA stands for locked nucleic acid and is defined by two sugar moieties containing a methylene bridge between the 2-oxygen and the 4-carbon of the ribofuranose ring a two nucleotide LNA-modified overhang on the 3′ site. Additionally the first nucleotide on the 5′ site of the sense strand is also LNA modified to facilitate strand discrimination in the RISC complex. LNA-moiety restricts the flexibility of the monomer and locks it in a rigid bicyclic N-type conformation conferring exceptional tolerance against nucleases and extremely low cellular toxicity. Moreover, these minimal modifications provide a compromise between stability and functionality both form vitro and in vivo applications (Elme'n et al., 2005; Mook et al., 2007 as cited in Vinikov et al). LNA modifications will lead to greater melting temperatures (Tm values) for hybridization with complimentary sequences. Each LNA modified nucleotide can increase Tm up to 8° C. of a formed nucleotide pair (DOI: 10.1007/3-540-27262-3_21). The length of the sense- and anti-sense strand typically comprises 20-22, 20-23, 20-24 or 20-25 nucleotides.

Another alternative is to design the microRNA mimetics in the triple RNA strand design described under point 1.11 (Functional characterization of miRNAs in cellular assays).

If LNP particles are used for delivery, the dose might be between 0.01 and 5 mg/kg of the mass of miRNA mimetics per kg of subject to be treated, preferably 0.03 and 3 mg/kg, more preferably 0.1 and 0.4 mg/kg, most preferably 0.3 mg/kg. The administration of the LNP particles is preferably systemic, more preferably intravenous.

### EXAMPLES

1. Materials and Methods

1.1 AAV Production, Purification and Quantification

HEK-293h cells were cultivated in DMEM+GlutaMAX media supplemented with 10% fetal calf serum. Three days before transfection, the cells were seeded in 15 cm tissue culture plates to reach 70-80% confluency on the day of transfection. For transfection, 0.5 μg total DNA per cm² of culture area were mixed with 1/10 culture volume of 300 mM CaCl₂) as well as all plasmids required for AAV production in an equimolar ratio. The plasmid constructs were as follows: One plasmid encoding the AAV6.2 cap gene (Strobel B et al., 2015); a plasmid harboring an AAV2 ITR-flanked expression cassette containing a CMV promoter driving expression of a codon-usage optimized murine Tgfb1 gene and a hGh poly(A) signal, whereby the Tgfb1 sequence contains C223S and C225S mutations that increase the fraction of active protein (Brunner A M et al., 1989); a pHelper plasmid (AAV Helper-free system, Agilent). For GFP and stuffer control vector production, the Tgfb1 plasmid was exchanged for an eGFP plasmid, harboring an AAV2 ITR-flanked CMVeGFP-SV pA cassette and AAV-stuffer control plasmid, containing an AAV2 ITR-flanked non-coding region derived from the 3′-UTR of the E6-AP ubiquitin-protein ligase UBE3A followed by a SV40 poly(A) signal, respectively.

The plasmid CaCl₂) mix was then added dropwise to an equal volume of 2× HBS buffer (50 mM HEPES, 280 mM NaCl, 1.5 mM Na₂HPO₄), incubated for 2 min at room temperature and added to the cells. After 5-6 h of incubation, the culture medium was replaced by fresh medium. The transfected cells were grown at 37° C. for a total of 72 h. Cells were detached by addition of EDTA to a final concentration of 6.25 mM and pelleted by centrifugation at room temperature and 1000× g for 10 min. The cells were then resuspended in “lysis buffer” (50 mM Tris, 150 mM NaCl, 2 mM MgCl₂, pH 8.5). AAV vectors were purified essentially as previously described (Strobel B et al., 2015): For iodixanol gradient based purification, cells harvested from up to 40 plates were dissolved in 8 mL lysis buffer. Cells were then lysed by three freeze/thaw cycles using liquid nitrogen and a 37° C. water bath. For each initially transfected plate, 100 units Benzonase nuclease (Merck) were added to the mix and incubated for 1 h at 37° C. After pelleting cell debris for 15 min at 2500× g, the supernatant was transferred to a 39 mL Beckman Coulter Quick-Seal tube and an iodixanol (OptiPrep, Sigma Aldrich) step gradient was prepared by layering 8 mL of 15%, 6 mL of 25%, 8 mL of 40% and 5 mL of 58% iodixanol solution diluted in PBS-MK (lx PBS, 1 mM MgCl₂, 2.5 mM KCl) below the cell lysate. NaCl had previously been added to the 15 % phase at 1 M final concentration. 1.5 μL of 0.5% phenol red had been added per mL to the 15% and 25% iodixanol solutions and 0.5 μL had been added to the 58% phase to facilitate easier distinguishing of the phase boundaries within the gradient. After centrifugation in a 70Ti rotor for 2 h at 63000 rpm and 18° C., the tube was punctured at the bottom. The first five milliliters (corresponding to the 58% phase) were then discarded, and the following 3.5 mL, containing AAV vector particles, were collected. PBS was added to the AAV fraction to reach a total volume of 15 mL and ultrafiltered/concentrated using Merck Millipore Amicon Ultra-15 centrifugal filter units with a MWCO of 100 kDa. After concentration to ˜1 mL, the retentate was filled up to 15 mL and concentrated again. This process was repeated three times in total. Glycerol was added to the preparation at a final concentration of 10%. After sterile filtration using the Merck Millipore Ultrafree-CL filter tubes, the AAV product was aliquoted and stored at −80° C.

1.2 Mouse Models and Functional Readouts

For reporter gene studies, 9-12 week old female C57Bl/6 or Balb/c mice, purchased from Charles River Laboratories, either received 2.9×10¹⁰ vector genomes (vg) of AAV5-CMV-fLuc or 3×10¹¹ vg of AAV6.2-CMV-GFP, respectively, by intratracheal administration under light anesthesia (3-4% isoflurane). Alternatively, C57Bl/6 mice received 3×10¹¹ vg of AAV2-L1-CMV-GFP by intravenous (i.v.) administration. Two to three weeks after AAV administration (see figure descriptions), reporter readouts were performed. For luciferase imaging, mice received 30 mg/kg luciferin as a substrate via intraperitoneal administration prior to image acquisition. In the case of GFP reporters, either histological fresh-frozen lung sections were prepared and analyzed for direct GFP fluorescence by fluorescence microscopy or formalin-fixed paraffin embedded slices were prepared for GFP IHC analysis (see detailed description further below).

For the fibrosis models, male 9-12 week old C57Bl/6 mice purchased from Charles River Laboratories received intratracheal administration of either 2.5×10¹¹ (vg) of AAV-TGFβ1 or AAV-stuffer, 1 mg/kg Bleomycin or physiological NaCl solution in a volume of 50 μL, which was carried out under light anesthesia. Fibrosis was assessed at day 3, 7, 14, 21 and 28 after AAV/Bleomycin administration. Briefly, to assess lung function, mice were anesthetized by intraperitoneal (i.p.) administration of pentobarbital/xylazine hydrochloride, cannulated intratracheally and treated with pancuronium bromide by intravenous (i.v.) administration. Lung function measurement (i.e. lung compliance, forced vital capacity (FVC)) was then conducted using the Scireq flexiVent FX system. Mice were then euthanized by a pentobarbital overdose, the lung was dissected and weighed prior to flushing with 2×700 μL PBS to obtain BAL fluid for differential BAL immune cell and protein analyses (data not shown). The left lung of each mouse was processed for histological assessment by a histopathologist, whereas the right lung was used for total RNA extraction, as detailed below.

For the miR-212-5p AAV pharmacokinetic study we used male, C57BL/6JRj mice, 10-12 weeks old from Janvier Labs. Mice were intratrachealy (i.t.) instilled with stuffer negative control AAV (1×10¹¹ vg) or three rising dosages (9×10⁹ vg, 10×10¹⁰ vg and 1×10¹¹ vg) of miR-212-5p-AAV. it. instillation was carried out under light anesthesia with short exposure to isoflurane. Mice were euthanized on day 7, day 14 and day 28 after AAV instillation. Lungs were snap frozen in liquid nitrogen and processed to frozen lung powder for total RNA isolation (using miRNAeasy kit from Qiagen).

1.3 Histology

For the preparation of histological lung samples, the left lung lobe was mounted to a separation funnel filled with 4% paraformaldehyde (PFA) and inflated under 20 cm water pressure for 20 minutes. The filled lobe was then sealed by ligature of the trachea and immersed in 4% PFA for at least 24 h. Subsequently, PFA-fixed lungs were embedded in paraffin. Using a microtome, 3 μm lung sections were prepared, dried, deparaffinized using xylene and rehydrated in a descending ethanol series (100-70%). Masson's trichrome staining was performed using the Varistain Gemini ES Automated Slide Stainer according to established protocols. For GFP-IHC, enzymatic antigen retrieval was performed and antibodies were diluted at indicated ratios in Bond primary antibody diluent (Leica Biosystems). Slides were stained with the 1:1000 diluted polyclonal Abcam rabbit anti-GFP antibody ab290 and appropriate isotype control antibodies, respectively. Slides that had only received antigen retrieval served as an additional negative control. Finally, sections were mounted with Merck Millipore Aquatex medium.

1.4 RNA Preparation

For total lung RNA preparation, the right lung was flash frozen in liquid nitrogen immediately after dissection. Frozen lungs were homogenized in 2 mL precooled Qiagen RLT buffer+1% β-mercaptoethanol using the Peqlab Precellys 24 Dual Homogenizer and 7 mL-ceramic bead tubes. 150 μL homogenate were then mixed with 550 μL QIAzol Lysis Reagent (Qiagen). After addition of 140 μL chloroform (Sigma-Aldrich), the mixture was shaken vigorously for 15 sec and centrifuged for 5 min at 12,000×g and 4° C. 350 μL of the upper aqueous RNA-containing phase were then further purified using the Qiagen miRNeasy 96 Kit according to the manufacturer's instructions. After purification, RNA concentration was determined using a Synergy HT multimode microplate reader and the Take3 module (BioTek Instruments). RNA quality was assessed using the Agilent 2100 Bioanalyzer.

1.5 RNA Sequencing

cDNA libraries were prepared using the Illumina TruSeq RNA Sample Preparation Kit. Briefly, 200 ng of total RNA were subjected to polyA enrichment using oligo-dT-attached magnetic beads. PolyA-containing mRNAs were then fragmented into pieces of approximately 150-160 bp. Following reverse transcription with random primers, the second cDNA strand was synthesized by DNA polymerase I. After an end repair process and the addition of a single adenine base, phospho-thymidine-coupled indexing adapters were coupled to each cDNA, which facilitate sample binding to the sequencing flow cell and further allows for sample identification after multiplexed sequencing. Following purification and PCR enrichment of the cDNAs, the library was diluted to 2 nM and clustered on the flow cell at 9.6 pM, using the Illumina TruSeq SR Cluster Kit v3-cBot-HS and the cBot instrument. Sequencing of 52 bp single reads and seven bases index reads was performed on an Illumina HiSeq 2000 using the Illumina TruSeq SBS Kit v3-HS. Approximately 20 million reads were sequenced per sample.

For miRNA, the Illumina TruSeq Small RNA Library Preparation Kit was used to prepare the cDNA library: As a result of miRNA processing by Dicer, miRNAs contain a free 5′-phosphate and 3′-hydroxal group, which were used to ligate specific adapters prior to first and second strand cDNA synthesis. By PCR, the cDNAs were then amplified and indexed. Using magnetic Agencourt AMPure XP bead-purification (Beckman Coulter), small RNAs were enriched. The samples were finally clustered at 9.6 pM and sequenced, while being spiked into mRNA sequencing samples.

1.6 Computational Processing and Data Analysis (mRNA-Seq and miRNA-Seq Data Processing)

mRNA-Seq reads were mapped to the mouse reference genome GRCm38.p6 and Ensembl mouse gene annotation version 86 (http://oct2016.archive.ensembl.org) using the STAR aligner v. 25.2a (Dobin et al., 2013). Raw sequence read quality was assessed using FastQC v0.11.2, alignment quality metrics were checked using RNASeQC v1.18 (De Luca D. S. et al., 2012). Subsequently, duplicated reads in the RNA-Seq samples were marked using bamUtil v1.0.11 and subsequently duplication rates assessed using the dupRadar Bioconductor package v1.4 (Sayols-Puig, S. et al., 2016). Read count vectors were generated using the feature counts package (Liao Y. et al., 2014). After aggregation to count matrices data were normalized using trimmed mean of M-values (TMM) and voom transformed to generate log(counts per million) (CPM) (Ritchie M. E., 2015). Descriptive analyses such as PCA and hierarchical clustering were carried out to identify possible outliers. Differential expression between treatment and respective controls at each time points were carried out using limma with a significance threshold of p adj≤0.05 and abs(log ₂FC)≥0.5. Two samples out of 124 in total were excluded for not passing QC criteria.

miRNA-Seq reads were trimmed using the Kraken package v.12-274 (Davis M. P. A. et al., 2013) and subsequently mapped to the mouse reference genome GRCrn38.p6 and the miRbase v. 21 mouse miRNA (http://mirbase.org) using the STAR aligner v. 2.5.2a. Raw sequence read quality was assessed using FastQC v0.11.2 (http://www.bioinformatics.babraham.ac.uk/projects/fastqc/), trimming size and biotype distribution assessed using inhouse scripts. After aggregation to count matrices data were normalized using trimmed mean of M-values (TMM) and voom transformed to generate log(counts per million) (CPM). Descriptive analyses such as PCA and hierarchical clustering were carried out to identify possible outliers. Differential expression between treatment and respective controls at each time points were carried out using limma with a significance threshold of p adj≤0.05 and abs(log 2FC)≥0.5.

1.7 Integrated Data Analysis (Correlation of Functional Parameters and Expression)

Spearman's rho between the measured values for lung function and lung weight vs. the voom transformed log(CPM) of each miRNA and mRNA across all samples of both models and all time points.

1.8 Determination of Putative miRNA-mRNA Target Pairs

To determine mRNA targets of miRNAs, a stepwise approach has been carried out. First lowly expressed miRNAs and mRNAs were removed from the expression matrix. Subsequently the Spearman's rho was calculated between voom transformed log(CPM) of each miRNA vs. each mRNA across all samples of both models and all time points, using the corAndPvalue function from WGCNA v. 1.60 (Langfelder & Horvath, 2008) The set of correlation based putative miRNA-mRNA pairs is defined as all combinations with a correlation≤˜0.6. To add sequence based prediction of putative miRNA-mRNA pairs, all combinations with predictions in at least two out of five most cited miRNA target prediction algorithms (DIANA, Miranda, PicTar, TargetScan, and miRDB) available in the Bioconductor package miRNAtap v. 1.10.0/miRNAtap.db v. 0.99.10 (Pajak & Simpson, is 2016) were taken as sequence based pairs. The final set of miRNA-mRNA pairs is the intersection of anticorrelation based and sequence based interaction pairs, reducing the number of predictions significantly to a high-confidence subset.

1.9 Mouse-Human Conservation of miRNA Sequences

For all murine and human miRNAs from miRBase 21 seed regions (position 2 to 7) were extracted. For all combinations of murine and human miRNAs global alignments between the seed regions and the mature were calculated using the pairwiseAlignment function from the Bioconductor Biostrings package (v2.46.0). We applied the Needleman-Wunsch algorithm using an RNA substitution matrix with a match score of 1 and a mismatch score of 0. We assigned two categories to the miRNA candidates —“conserved” for miRNAs with an alignment score of 6 in the seed region for mouse-human pairs of miRNAs with the same name, “non-conserved” for miRNAs with an alignment score<6 in the seed region for mouse-human pairs of miRNAs with the same name. In addition, miRNAs with an alignment score for the alignment of the respective mature sequences above 20 is assigned to the category “mature high similarity”.

1.10 Characterization of miRNAs Based on Gene Set Enrichment of Target Gene Sets

The functional characterization of miRNAs is carried out using the enrichment function on the predicted mRNA targets from the MetabaseR package v. 4.2.3 and the gene set categories “pathway maps”, “pathway map folders”, “process networks”, “metabolic networks”, “toxicity networks”, “disease genes”, “toxic pathologies”, “GO processes”, “GO molecular functions”, “GO localizations”. The enrichment function performs a hypergeometric test on the overlap of the query gene set and the reference sets from Metabase. The data retrieval for the characterization of miRNA target sets was carried out on Metabase on Mar. 12, 2018.

1.11 Functional Characterization of miRNAs in Cellular Assays

miRNAs were characterized regarding their impact on the cellular production of the pro-inflammatory cytokine IL-6 and the pro-fibrotic processes fibroblast proliferation, fibroblasts-to-myofibroblasts transition (FMT), collagen expression and epithelial-to-mesenchymal transition (EMT). Unless stated differently in the Figures or Figure Legends, A549, NHBEC (normal human bronchial epithelial cells) or NHLF (normal human lung fibroblast) cells were transiently transfected with miRNA mimetic at a concentration of 2 nM for single miRNAs or 2+2 nM for miRNA combinations.

All miRNA mimetics used in the experiments shown in the Figures were purchased from Qiagen in the three stranded miRCURY LNA miRNA Mimic format. The design of miRCURY LNA miRNA Mimics includes three RNA strands, rather than the two RNA strands that characterize traditional miRNA mimics. The miRNA (guide) strand is an unmodified RNA strand with a sequence corresponding exactly to the annotation in miRBase. However, the passenger strand is divided into two LNA-enhanced RNA strands (https://www.qiagen.com/de/products/discovery-and-translational-research/functional-andcell-analysis/mirna-functional-analysis/mircury-lna-nirma-mimics/mircury-lna-mirna-mimics/#orderinginformation). When designed correctly, these triple RNA strand mimics are as potent as traditional double-strand RNA mimics. The great advantage is that the segmented nature of the passenger strand ensures that only the miRNA strand is loaded into the RNA-induced silencing complex (RISC) with no resulting miRNA activity from the two complementary passenger strands. Phenotypic changes observed with miRCURY LNA miRNA mimics can therefore be safely ascribed to the miRNA simulated by the mimic (see figure miRNA target identification with biotinylated mimics).

The distinct triple RNA strand design is enabled by incorporation of high-affinity LNA nucleotides into the two passenger strands. The sequence, length and LNA spiking pattern of the two passenger strands have been optimized using a sophisticated and empirically derived design algorithm. Bramsen, J. B., et al. (2007) Improved silencing properties using small internally segmented interfering RNAs. Nucleic Acids Research 35:5886-5897. PMID: 17726057. Griffiths-Jones, S. (2004) The miRNA Registry. Nucleic Acids Research Database Issue 32:D109-111. 3. miRBase: www.mirbase.org. Kahn, A. A., et al. (2009) Transfection of small RNAs globally perturbs gene regulation by endogenous miRNAs. Nature Biotechnology 27(6):549-555. doi: 10.1038/nbt.1543.

The miRNA mimetics for miR-29a-3p, miR-181a-5p and miR-212-5p and the corresponding control were used in FIGS. 22, 23 and 24:

hsa-miR-29a-3p: MIMAT0000086:
5′UAGCACCAUCUGAAAUCGGUUA
hsa-miR-181a-5p: MIMAT0000256:
5′AACAUUCAACGCUGUCGGUGAGU
hsa-miR-212-5p: MIMAT0022695:
5′ACCUUGGCUCUAGACUGCUUACU
negative control 4:
GAUGGCAUUCGAUCAGUUCUA

All other miRNA mimetics used for the other Figures were designed analogously. Thus, for miR-181b-5p a sequence of Seq ID No. 19 was used, and for miR-10a-5p a sequence according Seq ID No. 18 was used.

For the latter condition, 4 nM miRNA controls were used. Twenty-four hours later, TGFβ1 was added to the cells at 5 ng/mL concentration and cells were incubated for 24 h (IL-6, proliferation assays and collagen mRNA expression) or 72 h (collagen protein expression, FMT and EMT assays). For the measurement of gene expression, total RNA was extracted from the cells using the Qiagen RNeasy Plus 96 Kit and reversely transcribed into cDNA using the High-Capacity cDNA Reverse Transcription Kit (Thermo Fisher Scientific). IL-6 gene expression was detected by a Tagman qPCR assay (Hs00174131_ml). IL-6 protein was quantified in the cell supernatant using the MSD V-PLEX Proinflammatory Panel 1 Human kit. To assess cell proliferation, cells were grown in presence of TGFβ1 for 24 h and assayed using a WST-1 proliferation assay kit (Sigma/Roche). FMT was assessed by growing NHLF cells as described above, followed by fixation and fluorescent immunostaining of Collagen TaT. Images were taken using an IN Cell Analyzer 2000 high-content cellular imaging system and collagen was quantified and normalized to cell number (identified by DAPI-stained nuclei). EMT assessment relied on the same principle, however, using NHBEC cells and immuno-staining of E-cadherin.

Immunoblots were done according to standard methods using novex gels and according buffers from ThermoFisher and electrophoresis devices from BioRad. All primary antibodies were ordered from Cell Signaling Technology.

All cellular assays were performed with either primary lung epithelial cells or primary lung fibroblasts derived from human patient material. Thus, by the heterogeneity of each individual patient donor, e.g. its genetics, environment, cause of disease/surgery, cell isolation, etc., the derived cell also underlie a certain heterogeneity. Thus, it can happen that there are slight assay-to assay variabilities, which explain a certain standard deviation and different assay windows between equal assay formats. Nevertheless, we used primary cells because they are primary patient material and therefore more relevant for the human disease.

2. Results

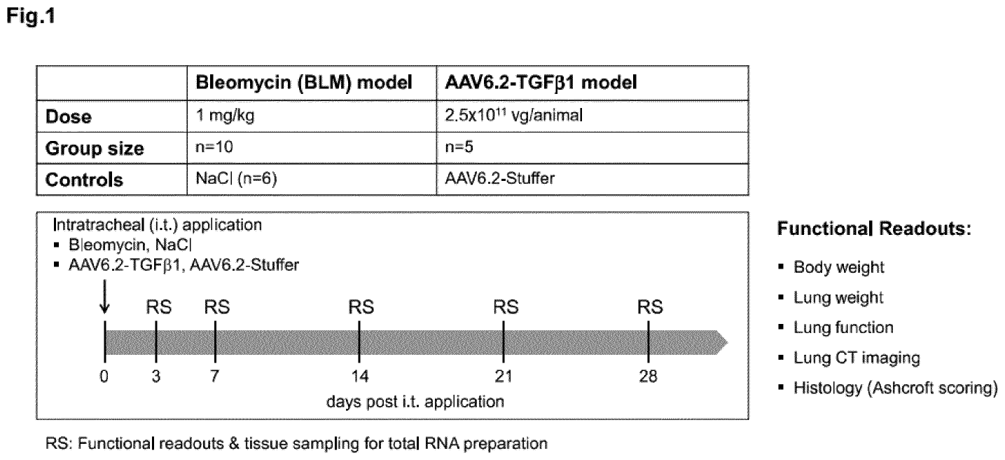

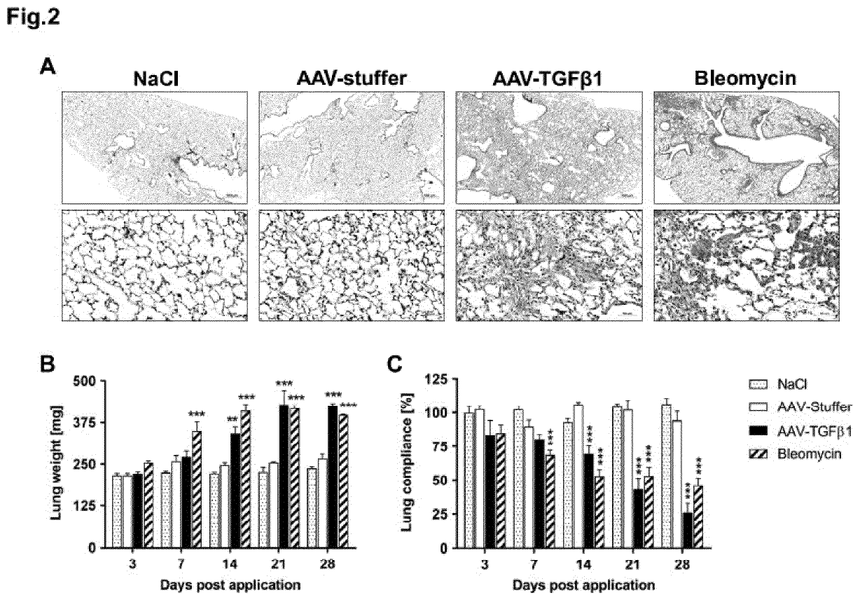

AAV-TGFβ1 and Bleomycin administration induce fibrosing lung pathology in mice. Following administration of either AAV-TGFβ1, Bleomycin or appropriate controls (NaCl, AAV-stuffer), longitudinal fibrosis development was measured over a time period of 4 weeks, as illustrated in FIG. 1. As evident from histological analysis of Massontrichrome stained lung tissue sections on day 21, a pulmonary fibrosis phenotype characterized by thickened alveolar septa, increased extracellular matrix deposition and presence of immune cells was evident in AAV-TGFβ1 and Bleomycin treated animals but absent in NaCl and AAV-stuffer control mice (FIG. 2). A strong increase in lung weight in diseased animals clearly confirmed aberrant ECM deposition and tissue remodeling. Moreover, as a functional consequence, lung function was significantly compromised following TGFβ1 overexpression and Bleomycin treatment, thereby mirroring clinical observations in patients with fibrosing ILDs. Notably, whereas Bleomycin-induced changes in functional readouts occurred about one week prior to the changes in the AAV-TGFβ1 model, a very similar phenotype was evident from day 21.

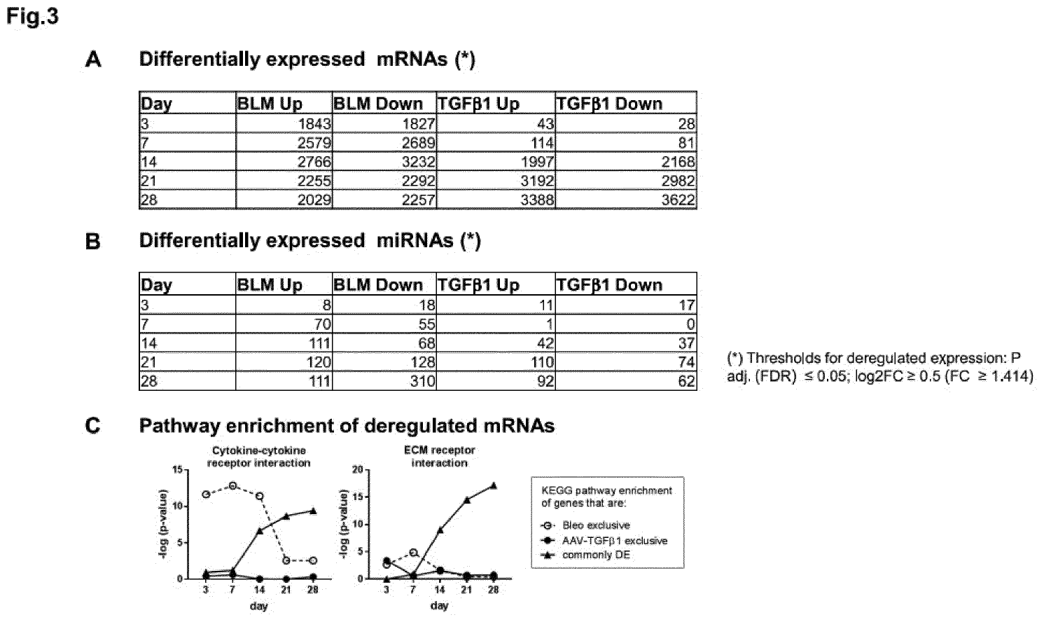

Transcriptional characterization of chronological disease manifestation. In order to dissect the molecular pathways and overall changes in gene expression underlying disease development and progression in the two models of pulmonary fibrosis, RNA was prepared from lung homogenates of each animal and applied to next generation sequencing (NGS) analysis. The number of differentially expressed mRNAs and miRNAs is depicted in FIG. 3. Pathway analysis (FIG. 3C) demonstrated expected enrichment for injury- and acute inflammation related processes at the early time points in the Bleomycin model, whereas inflammation was initially absent in the AAV model and only present during the stages of fibrosis development (day 14 onwards). In contrast, enrichment for remodeling/ECM-associated processes occurred in both disease models in a similar fashion, approximately from day 14 onwards.

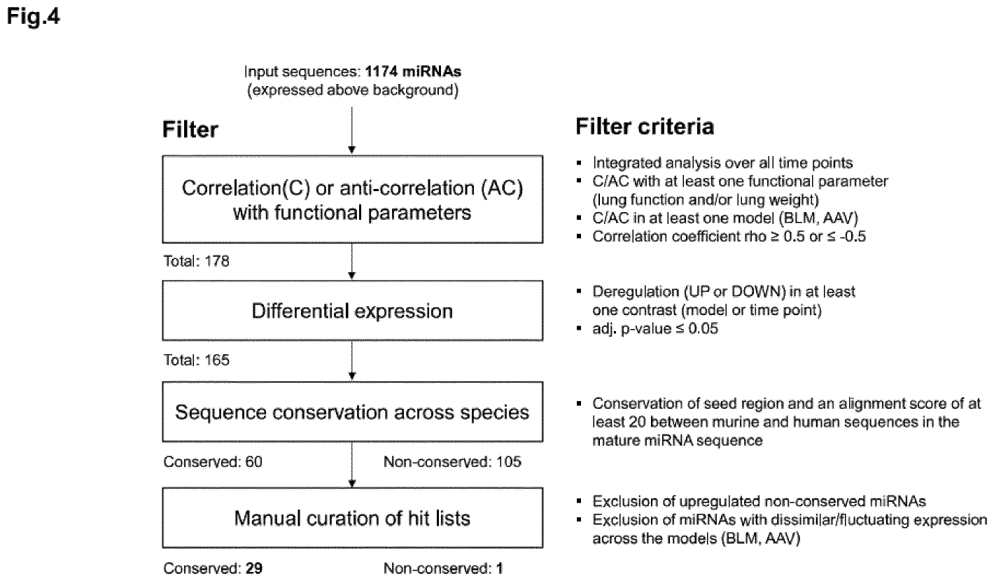

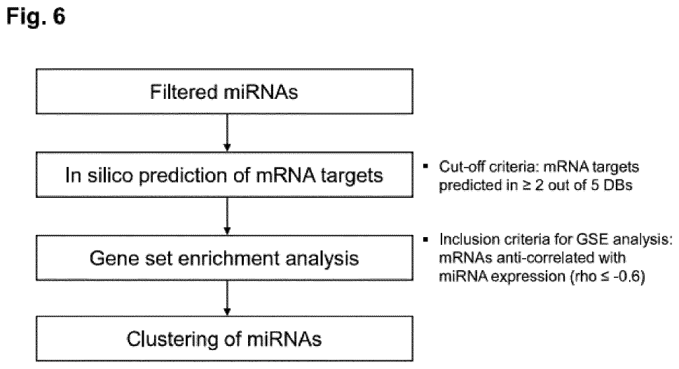

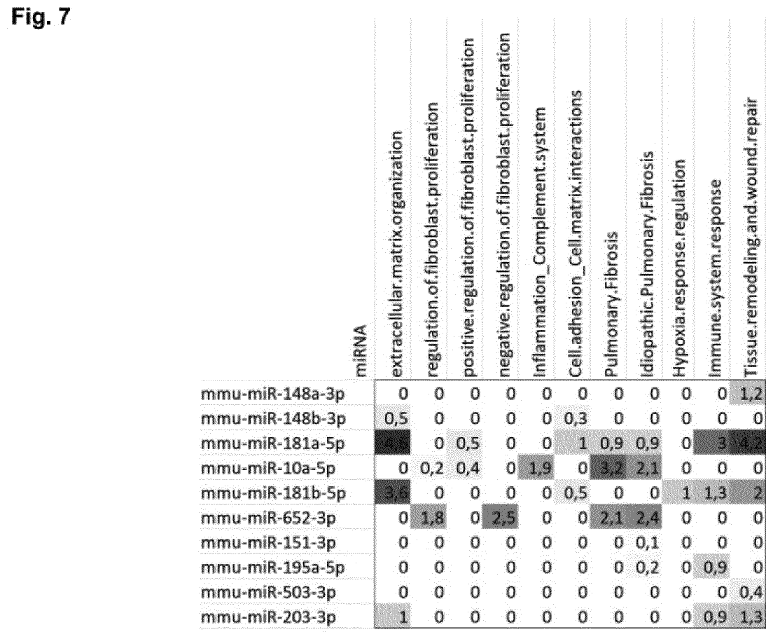

Identification of miRNAs associated with clinically relevant disease phenotypes. To identify candidate miRNAs likely to be directly associated with disease development, a staggered selection strategy using multiple filter criteria was set up (FIG. 4). The central aspect —fibrosis association—was incorporated by selecting only those miRNAs, whose longitudinal expression profiles either strongly correlated or anti-correlated with the observed decrease in lung function or increase in lung weight, respectively. Moreover, a candidate miRNA needed to be differentially expressed at least at one time point in one of the models. miRNAs were then classified according to their species conservation (conserved in humans vs. only present in mice), based on seed sequence and full sequence similarity. The resulting miRNA candidate list was finally hand-curated to dismiss candidates with dissimilar expression in the two disease models and/or fluctuating expression profiles as well as upregulated but non-conserved miRNAs, which could not be targeted in humans. We further eliminated miRNAs that, according to literature text mining results had been previously patented in the context of lung fibrosis. The final hit list is shown in FIG. 5. miRNA target prediction (FIG. 6). As an initial approach to characterize the functional role of the miRNAs, putative mRNA targets were predicted computationally, by querying DIANA, MiRanda, PicTar, TargetScan, and miRDB databases via the Bioconductor package miRNAtap (see materials & methods section for details). Targets that were predicted by at least two out of five databases were considered further. Each miRNA target gene set was then analyzed for enrichment of specific disease-relevant processes and FIG. 7 exemplarily illustrates putative functions of genes targeted by specific miRNAs.

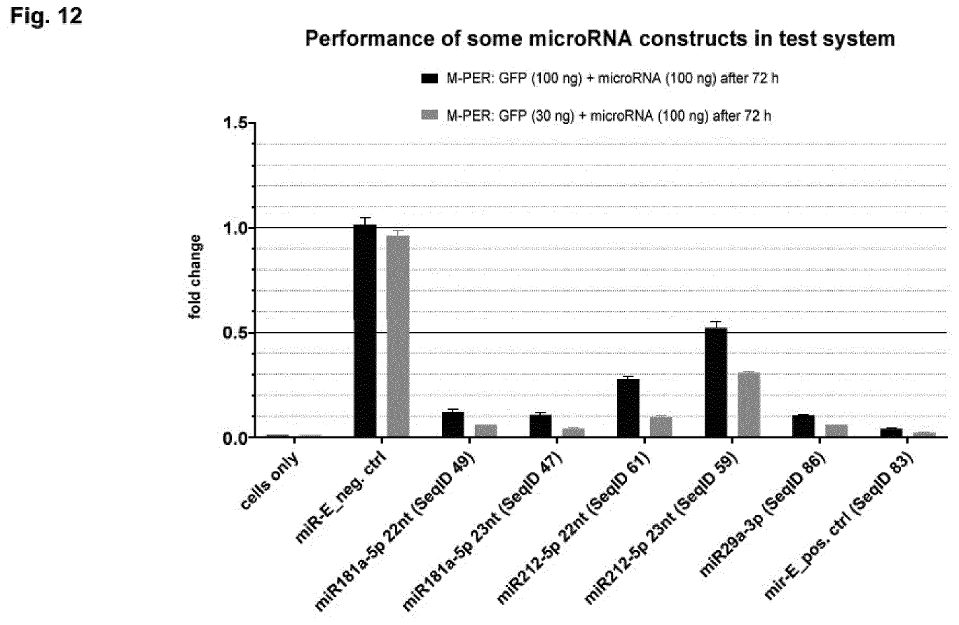

Functionality of miRNAs in mir-E backbone (FIG. 12). A GFP expression construct with target sequences for the miRNAs in the 3′UTR was used to demonstrate the functionality of the miRNA sequences in the mir-E backbone. HEK-293 cells were transiently transfected with the GFP expression construct in combination with a plasmid encoding one of the miRNAs. 72 h after transfection the GFP fluorescence was determined. The fluorescence signal of the negative control, i.e. a miRNA without target sequence in the 3′UTR of the GFP, was set to 100% and the fold change of the fluorescence signals of all other constructs were put into relation to the negative control. The positive control is an optimal mir-E construct and as expected leads to the most pronounced knock-down of GFP. All other construct also lead to a clear knock-down of GFP, indicating that they are not only properly expressed but also correctly processed. The optimal length of the guide strand in the mir-E backbone is 22 nucleotides (nt) which might explain why the miR212-5p with 23nt is not as efficacious as the one with only 22nt. Accordingly, a miR212-5p with 22nt is one preferred embodiment of the present invention.

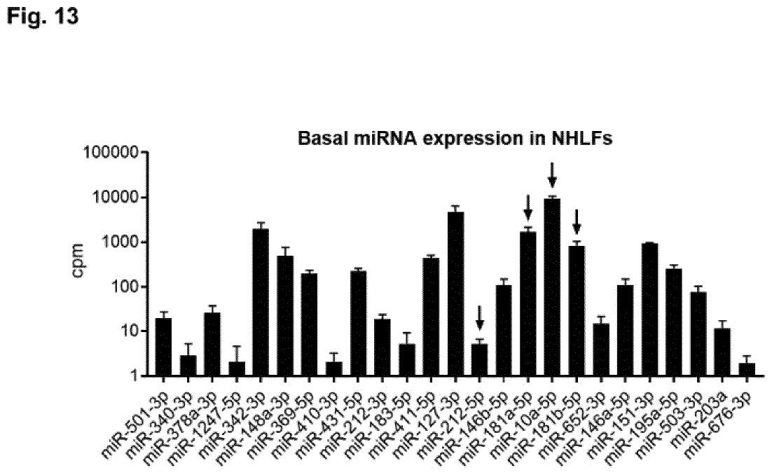

miRNA expression in primary human lung fibroblasts (FIG. 13). To analyze the expression of candidate miRNAs in the human context, small RNA sequencing was performed in primary human lung fibroblasts. As indicated in FIG. 13, robust expression, although at varying levels, was observed for all miRNAs from the candidate list, thereby supporting the concept of species translation of our findings in murine lung fibrosis models to humans.

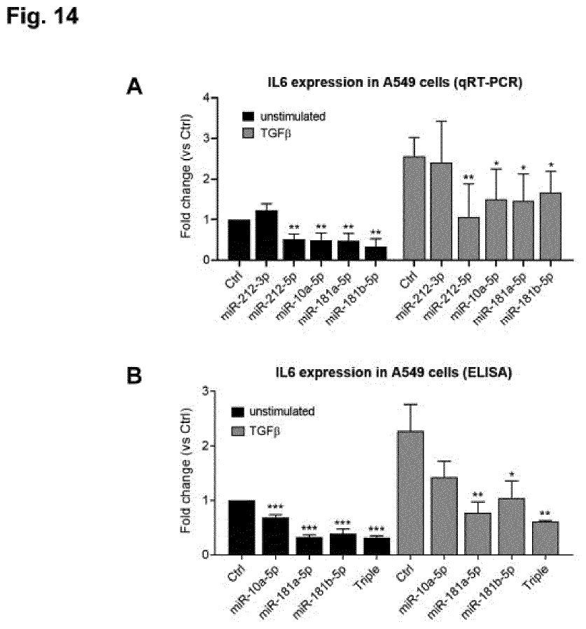

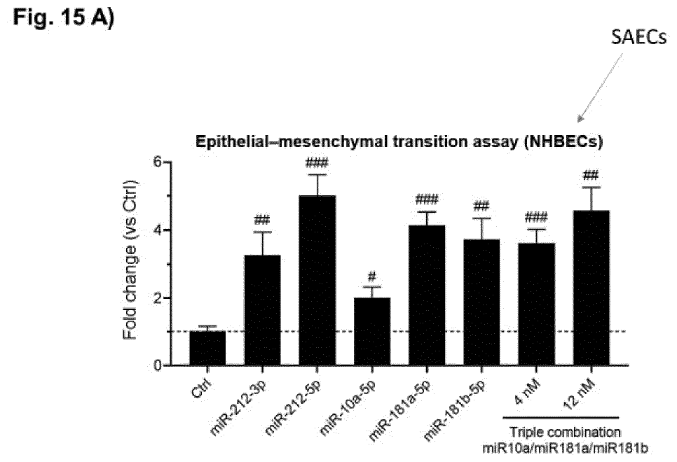

Functional characterization of miRNAs in cellular assays (FIGS. 14-21). To demonstrate anti-fibrotic functions of candidate miRNAs, synthetic miRNA mimetic comprising the fully matured miRNA sequences were generated to perform transient transfection experiments in cellular assays reflecting key mechanisms of fibrotic remodeling. In a first set of experiments the effect of five selected miRNAs (mir-10a-5p, mir-181a-5p, mir-181b-5p, mir-212-3p, mir-212-5p) was analyzed in A549 cells and in primary bronchial airway epithelial cells in the presence or absence of pro-fibrotic TGFβ stimulation. As indicated in FIG. 14 (A), transient transfection of four out of five miRNA mimetic resulted in a significant reduction of TGFβ-induced mRNA expression of IL6, a well described marker gene for inflammation. The only exception was mir-212-3p, which did not show a significant anti-inflammatory effect in this setting. Interestingly, the same result was obtained in unstimulated A549 cells. To further underscore these findings on the protein level, IL6 expression was measured in cell culture supematants by ELISA. In these experiments mir-10-5p, mir-181a-5p, mir-181b-5p and a triple combination of these miRNAs were investigated. As shown in FIG. 14 (B), all individual miRNAs as well as the triple combination showed significant reduction of IL6 expression in unstimulated and TGFβ-stimulated A549 cells, thereby confirming the anti-inflammatory effect of these miRNAs. Besides its pro-inflammatory function, TGFβ also plays a central role as an inducer of epithelial to mesenchymal transition (EMT), a hallmark of fibrotic remodeling in pulmonary fibrosis. During TGFβ-induced EMT, expression of the airway epithelial marker gene E-Cadherin is reduced due to conversion of an epithelial to a fibroblast-like (mesenchymal) cellular phenotype. To assess a potential protective role of selected miRNA candidates on TGFβ-induced EMT, a cellular assay using primary human airway epithelial cells in combination with high-content cellular imaging analysis for quantification of E-Cadherin expression was applied. As depicted in FIG. 15, all miRNAs tested in this setting showed pronounced inhibitory effects on TGFβ-mediated EMT induction, as demonstrated by significantly higher E-Cadherin expression levels in miRNA treated groups as compared to control groups.

Because we also wanted to assess other miRNA combinations, beyond miR-10a+miR181a-5p+miR-181b-5p, we repeated the former EMT assay, depicted in FIG. 15B. The single miR-181a-5p, miR-181b-5p and miR-212-5p were again able to restore E-cadherin protein expression after TGFβ treatment of lung epithelial cells. Also combinations of miR-181a-5p+miR-212-5p+miR10a-5p and combination of miR-181a-5p+miR-212-5p showed a significant improvement of E-cadherin expression in the EMT assay. Consistently, the best effects were observed with a triple combination of miR-181a-5p+miR-181b-5p+miR10a-5p, which allows a reduction of miRNA dosage to achieve similar effects that miR-181a-5p, miR-181b-5p or miR-10a-5p alone (FIG. 15B). Assay window variabilities between FIG. 15A and FIG. 15B, are explainable by slight assay-to-assay variabilities in combination with different behavior of primary human derived lung epithelial cells from different donors. Nevertheless, the direction of the miRNA effect and its significance stays the same.

In addition to airway epithelial cells, fibroblasts are considered as a highly relevant cell type for fibrotic processes. By acting as the main source for excessive production of collagen and other extracellular matrix components, fibroblasts directly contribute to lung stiffening associated with impaired lung function and finally loss of structural lung integrity. To further investigate the function of candidate miRNAs during fibroblast activation, transient transfection experiments were carried out in primary human lung fibroblasts under unstimulated and TGFβ-stimulated (pro-fibrotic) conditions. As functional readouts IL6 expression, collagen expression and fibroblast proliferation were assessed in absence or presence of miRNAs. As shown in FIG. 16, all miRNAs analyzed showed significant reduction of IL6 expression in the presence and absence of TGFβ as measured by qRT-PCR. Moreover, mir-212-3p, mir-181a-5p and mir-181b-5p showed inhibitory effects on fibroblast proliferation, both under basal as well as under TGFβ-induced conditions as illustrated in FIG. 17. As depicted in FIG. 18, only the triple combination of mir-10a5p, mir-181a-5p and mir-181b-5p showed a significant and dose-dependent effect on TGFβ-induced FMT compared to control groups, while none of the tested miRNAs showed significant effects when transfected individually. Nevertheless, miR-212-5p showed a trend wise reduction of collagens in this assay (FIG. 18) with this fibroblast donor. To elucidate whether the observed trend wise reduction of collagen deposition by miR212-5p could lead to a significant reduction and because assay variabilities can occur, by working with primary cells, the FMT assay was repeated with 7 different fibroblast donors and a wider range of miRNA dosages (FIG. 19).

FIG. 19 shows the effect of single miRNA 181a-5p and miR-212-5p on collagen 1 deposition upon TGFβ stimulation in a FMT assay. miR-181a-5p trend wise reduces collagen 1 deposition at higher concentrations. miR-212-5p significantly diminishes collagen 1 deposition of normal and IPF-lung fibroblasts, starting at 0.25 nM, in comparison to the respective miRNA control mimetic (FIG. 19). In addition to collagen 1 deposition, miR-181a5p and miR-212-5p affect also novel collagen expression in human lung fibroblasts beyond collagen 1 (FIGS. 20 and 21). When stimulated with TGFβ, miR-181a-5p and miR-212-5p reduced intracellular collagen 1a1 and collagen 5a1 (FIG. 20A/B). The combination of miR-181a-5p and miR-212-5p showed an additional significant reduction of collagen 1a1 protein expression in comparison to the miRNA negative control (FIG. 20A). In accordance to the reduction of Col1a1 and Col5a1, Col3a1 mRNA expression was also reduced significantly by miR-212-5p and the combination of miR-181a-5p and miR-212-5p (FIG. 20C). To finally validate that the observed anti-fibrotic effects of miR-181a-5p and miR212-5p on human lung fibroblasts are not (only) mediated via modulation of TGFβ signaling, miRNA mimetic were also tested in an epithelial-fibroblast co-culture, mimicking the cellular fibrotic niche (FIG. 21). In this co-culture system, where pro-fibrotic mediators from epithelial cells activates co-cultured human lung fibroblast, miR-212-5p reduced Col1a1 expression significantly in the human lung fibroblasts, independently of a pre-stimulation of epithelial cells with TGFβ (FIG. 21).

FIG. 22 shows the effect of single miRNA-29a-3p, miRNA-181a-5p and miR-212-5p and its combinations on collagen 1 deposition upon TGFβ stimulation in a FMT assay. miR-29a-3p significantly reduced collagen deposition up to 50% and miR-212-5p reduced collagen deposition significantly up to 78%. miR-181a-5p showed a trend wise reduction of collagen which could be improved by the combination with miR-29a, leading at higher concentrations also to a 50% reduction. Combining miR-29a-3p with miR-212-5p resulted in a significant reduction of collagen up to 80%. Of note, this could be achieved with half of the miRNA dose for each particular miRNA of the combination compared to the dose of the single miRNAs for miRNA-29a-3p or miR-212-5p. Even using only 1.33 nM of each miRNA, miR-29a-3p, miR-212-5p and miR-181a-5p, in a triple combination a collagen reduction of app. 70% could be achieved significantly in comparison to the miRNA control (FIG. 22).

In accordance to collagen 1 deposition in primary lung fibroblasts, miR-181a-5p and miR212-5p profoundly inhibit intracellular collagen 1 synthesis, especially when they were dosed in combination (FIG. 23A, 24). This reduction of Col1a1 protein synthesis of app 50% could be significantly improved by adding miR-29a-3p to the dual miR-181a-5p/miR212-3p combination, resulting in a full inhibition of Col1a1 synthesis. For Col5a1 (FIG. 23B, 24) protein synthesis and Col3a1 RNA de novo synthesis (FIG. 23C, 24), equal trends could be observed with the triple combination of miR-29a-3p, miR-181a-5p and miR-212-5p, showing a more robust inhibition of these collagen subtypes after TGFβ stimulation.

To characterize the performance of a viral construct, we transduced naive mice with increasing dosages of an AAV 6.2 construct, containing a miR-212-5p expression cassete (see Seq ID NO: 61, derived from plasmid according to Seq ID No: 91, see FIG. 26) and sacrificed theses mice on day 7, 14 and 28 after AAV intratracheal lung instillation. As shown in FIG. 25, increasing dosages of the miR-212-5p AAV leads to a dose dependent increase in miR-212-5p lung expression (FIG. 25). On day 7, 1×10¹¹ vg f the miR-212-5p AAV resulted in a 300 fold up-regulation of miR-212-5p, which could be elevated to a 350 fold increase on the later time points 14d and 28d.

In summary, the functional characterization in human airway epithelial cells and human lung fibroblasts demonstrates anti-inflammatory, anti-proliferative and anti-fibrotic effects for selected miRNA candidates. The most pronounced effects across all assay formats were observed for miR-181a-5p, mir-181b-5p and mir-212-5p, whereas mir-10a-5p and mir212-3p showed similar profiles although at weaker efficiency compared to the aforementioned miRNAs. In the FMT assay we observed positive effects by miR-10a-5p, miR-181a5p, miR-181b-5p and miR-212-5p, whereas a triple combination of mir-10a-5p, mir-181a5p and mir-181b-5p showed an improved inhibitory effects in the FMT assay, indicating an additive or synergistic effect for this combination. Overall we observed a very potent antifibrotic effect of miR-181a-5p on lung epithelial cells and a very potent anti-fibrotic effect of miR-212-5p on fibroblasts, which suggests that the combination of these two miRNAs are very potent anti-fibrotic combination affecting the two most important cell types in pulmonary fibrosis. Therefore, combinations of miRNA candidates, and especially mimetics of miR-181a-5p and miR-212-5p or its respective mimetics, provide a preferred option for the development of therapeutic approaches with superior efficiency profiles compared to single miRNAs. In addition, we were able to validate the published collagen inhibitory effects of single miR-29a-3p under fibrotic conditions. By specific combination of miR29a-3p with miR-212-5p in a dual combination or with miR-212-5p and miR-181a-5p in a triple combination, we could show that this leads to an even more pronounced anti-fibrotic effect compared to the single miRNAs or the dual combination of miR-212-5p with miR181a-5p. By using lower doses of the involved miRNAs in the combinations, compared to their single use, we are able to keep their anti-fibrotic effects and will likely lead to reduced unwanted/unspecific effects in transduced cells. Furthermore, the specific combination of mirR-29a-3p with either miR-212-5p or with both miR-212-5p and miR-181a-5p would allow to potentially address pulmonary hypertension (PH) in ILD, PF-ILD or IPF patients that either already have a PH co-morbidity or would otherwise develop one. It was shown by Chen, T. et al. that miR-212-5p increase could reduce RVSP and pulmonary vessel wall remodeling in a mouse model of pulmonary hypertension. Besides (super)additive or synergistical advantages the triple combination also combines anti-fibrotic effects on two key cell types in the pathogenesis of lung fibrosis: epithelial cells and fibroblasts. By combining miR-181a-5p, which has a very pronounced anti-fibrotic effect on the transformation of lung epithelial cells, and miR-212-5p and miR29a-3p, which possess massive anti-fibrotic effects on fibroblast activation and inhibition of ECM deposition, the triple combination of these three miRNAs increases the biological therapeutic spectrum against the single miRNAs.

Therapeutic applications of miRNAs. To translate the discovery of novel lung-fibrosis associated miRNAs into therapeutic applications, approaches based on vector-mediated expression offer an attractive opportunity for chronic diseases like pulmonary fibrosis by enabling long-lasting expression of miRNAs or miRNA-targeting sequences. As illustrated in FIG. 8, different vector design strategies are available to modulate miRNA function. For supplementation of miRNAs, which are downregulated under fibrotic conditions, vectors using Polymerase-II promoters (e.g. CMV, CBA) or Polymerase-III promoters (e.g. U6, H1) can be applied for the expression of a single miRNA sequence or a combination of is several miRNAs (FIG. 8A). While both promoter classes are generally amenable for miRNA expression, Polymerase-II promoter based constructs offer an additional advantage by enabling the use of cell-type-specific promoters thus allowing for the design of more specific and potentially safer vector constructs. Endogenous miRNAs are expressed as precursor molecules, so-called pri-miRNAs, which are first processed via the cellular RNAi machinery into pre-miRNAs and in a second step into the mature and biologically active form. To ensure efficient maturation of vector-derived miRNAs, a sequence of interest can be either expressed as endogenous pre-cursor miRNA or as an artificial miRNA by embedding a mature miRNA sequence into a foreign miRNA backbone like e.g. the miR30 scaffold or an optimized version thereof, the so-called miR-E backbone (Fellmann C et al., 2013). Examples for constructs which are based on the miR-E backbone are provided in the below example part and in the sequence listing. The constructs with a note “guide positions” are preferred (Table 1). In Seq ID No. 40-81 examples for the design of miRNA expression cassettes using the miR-E backbone are provided. While in Seq ID No. 40-69 examples for expression cassettes composed of mature miRNAs or natural pre-miRNAs are described for individual miRNAs, Seq ID No. 70-81 describe combinations of three different miRNAs in a mono-cistronic expression cassette. All expression cassettes provided, which are embedded in an AAV vector backbone, consist of inverted terminal repeats derived from AAV2, a CMV promoter, a SV40 poly adenylation signal and in some cases the enhanced green fluorescence protein (eGFP) gene upstream of the miRNA sequence(s). To modulate the functionality of miRNAs, which are upregulated under fibrotic conditions, two different vector design strategies can be applied, as described in FIGS. 8 B and C. 1) Expression of antisense-like molecules designed to specifically bind to pro-fibrotic miRNAs and thereby inhibit their function (FIG. 8B). Respective molecules, so called anti-miRs, can be incorporated into expression vectors as short hairpin RNAs (shRNAs) or as artificial miRNAs. In analogy to the miRNA supplementation approach, several miRNA-targeting sequences may be combined in a single vector, thereby enabling inhibition of various target miRNAs. 2) Expression of mRNAs containing several copies of miRNA binding sites, so called sponges, aiming to selectively sequester pro-fibrotic miRNAs and thereby inhibit their function (FIG. 8C). In summary, various vector design strategies are available for functional modulation (supplementation or inhibition) of lung-fibrosis associated miRNAs.

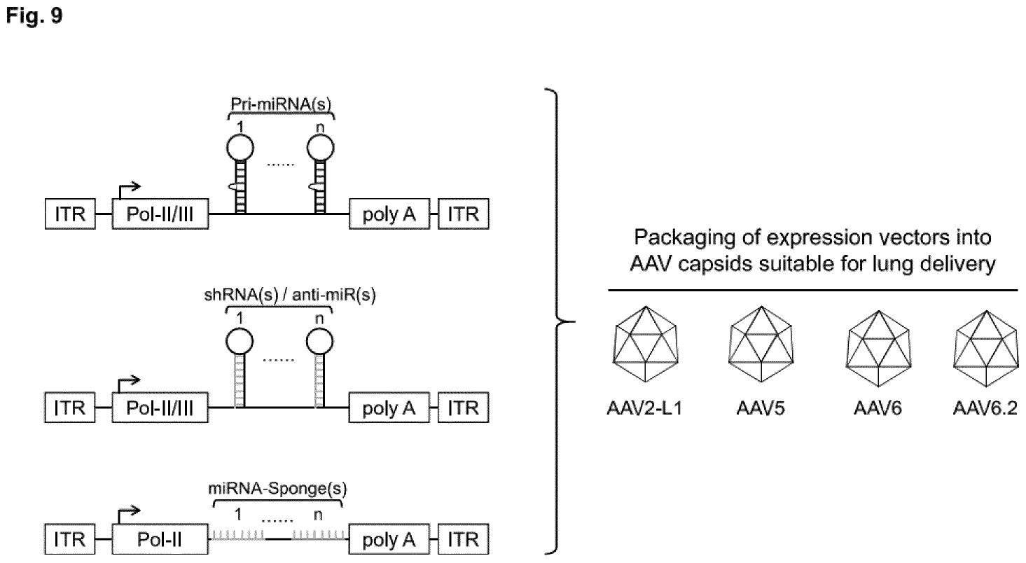

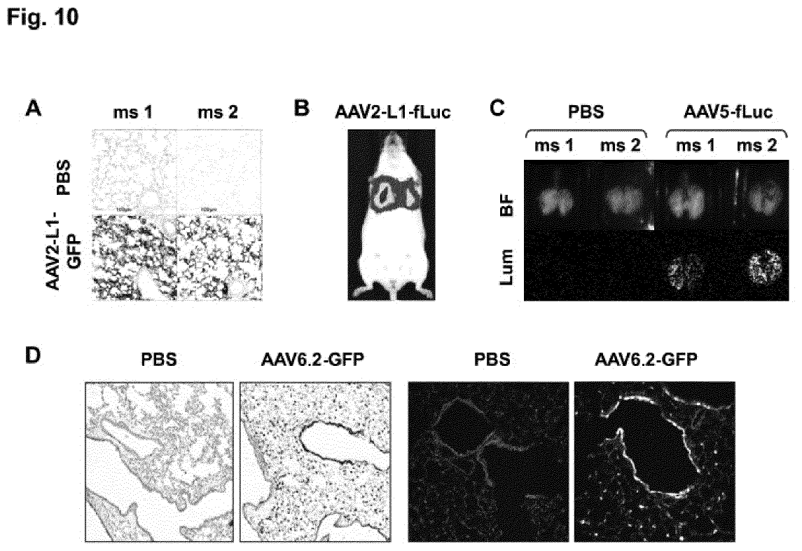

For the delivery of the aforementioned expression constructs to the lung non-viral as well as viral gene therapy vectors can be applied. However, compared to currently available non-viral delivery systems like e.g. liposomes, viral vectors demonstrate superior properties with regard to efficacy and tissue/cell-type selectivity, as demonstrated in various publications over the past years. Moreover, viral vectors offer great potential for engineering approaches to further improve potency, selectivity and safety properties. In recent years, viral vectors based on Adeno-associated virus (AAV) have emerged as one of the most favorable vector systems for in vivo gene therapy based on their excellent pre-clinical and clinical safety profile combined with highly efficient and stable gene delivery to various target organs and cell-types including fully differentiated and non-dividing cells. Since the discovery of the prototypic AAV serotype AAV2 in 1965 (Atchison et al.), various additional serotypes have been isolated from humans, non-human primates and from phylogenetically distinct species such as pigs, birds and others. To date more than 100 natural AAV isolates have been described, which interestingly differ with regard to tissue tropism. By applying capsid engineering approaches the repertoire of available AAV vectors for gene therapy approaches has been further expanded in recent years. Based on a landmark paper by Limberis et al. (2009), in which a systematic comparison of 27 AAV capsid variants and natural serotypes regarding lung transduction is described, AAV5, AAV6 and AAV6.2 were identified as highly suitable capsids for lung delivery following local routes of administration (e.g. intransal or intratracheal instillation). In addition, an engineered AAV capsid variant based on AAV2 (AAV2-L1) has been described recently as a novel vector enabling specific gene delivery to the lung after systemic vector administration (Körbelin et al., 2016). As described in FIG. 9, expression vectors containing miRNA-or miRNA-targeting sequences can be flanked by AAV inverted terminal repeats (ITRs) at the 5′- and the 3′-end, thereby enabling packaging of respective constructs into AAV capsids suitable for lung delivery, as exemplified by AAV2-L1, AAV5, AAV6 and AAV6.2. The potency of AAV-mediated lung delivery using the aforementioned capsid variants was confirmed in mouse studies by using reporter gene expressing constructs (GFP, fLuc) and subsequent assessment of transgene expression by immunohistochemistry (FIG. 10A,D) or in vivo imaging (FIG. 10B,C). On the histological level bronchial airway epithelial cells, alveolar epithelial cells and parenchymal cells were positively stained for reporter gene expression, indicating successful gene delivery to these cell types. Moreover, in the case of systemically delivered AAV2-L1 quantitative transgene expression was additionally detected in lung endothelial cells. Of note, transgene expression was stable with no decline of expression levels up to six months after the initial vector administration (data not shown). In summary AAV vectors represent a highly attractive delivery system for stable expression of therapeutic miRNAs or miRNA-targeting sequences in disease-relevant cell types of the lung thereby offering a novel and highly innovative multi-targeted treatment approach for IPF and other fibrosing interstitial lung diseases with a high unmet medical need.

### LIST OF REFERENCES

Adegunsoye A, Oldham J M, Fernández Pérez E R et al. Outcomes of immunosuppressive therapy in chronic hypersensitivity pneumonitis. ERJ Open Res. 2017; 3:00016-2017
Atchison R W, Casto B C, Hammon W M; Adenovirus-associated defective virus particles; Science. 1965 Aug. 13; 149(3685):754-6
Bagnato G. Harari S. Cellular interactions in the pathogenesis of interstitial lung diseases. Eur Respir Rev 2015; 24:102-114
Beckett. T Inhalation of Nebulized Perfluorochemical Enhances Recombinant Adenovirus and Adeno-Associated Virus-Mediated Gene Expression in Lung Epithelium, Human Gene Therapy Methods 23:98-110 (2012), DOI:10.1089/hgtb.2012.014
Brunner A M, Marquardt H, Malacko A R, Lioubin M N, Purchio A F (1989) Site-directed mutagenesis of cysteine residues in the pro region of the transforming growth factor beta 1 precursor. Expression and characterization of mutant proteins; J Biol Chem 264(23):13660-13664
Chen, T., MiR-212-5p Is a Potential Therapeutic Tool in Treatment of Pulmonary Hypertension Am J Respir Crit Care Med 2018; 197:A4616, www.atsjoumals.org
Chen, T., Engineering of Endothelium-Derived Extracellular Vesicles with Altered miRNA Cargo to Treat Severe Pulmonary Hypertension, Am J Respir Crit Care Med 2019; 199:A2401
Cottin V, Wollin L, Fischer A, Quaresma M, Stowasser S, Harari S. Fibrosing interstitial lung diseases: knowns and unknowns. Eur Resp Rev 2019; 28:180100
Davis M P, van Dongen S, Abreu-Goodger C, Bartonicek N, Enright A J; Kraken: a set of tools for quality control and analysis of high-throughput sequence data; Methods, 63 (1), (2013), pp. 41-49
DeLuca D S, Levin J Z, Sivachenko A, Fennell T, Nazaire M D, Williams C, Reich M, Winckler W, Getz G; RNA-SeQC: RNA-seq metrics for quality control and process optimization. Bioinformatics. 2012 Jun. 1; 28(11):1530-2
Dobin A, Davis C A, Schlesinger F, Drenkow J, Zaleski C, Jha S, Batut P, Chaisson M, Gingeras T R; STAR: ultrafast universal RNA-seq aligner. Bioinformatics. 2013 Jan. 1; 29(1):15-21
Doyle T J, Dellaripa P F. Lung manifestations in the rheumatic diseases. Chest 2017; 152:1283-95
Elme'n J, Thonberg H, Ljungberg K, Frieden M, Westergaard M, Xu Y, Wahren, B, Liang Z, ørum H, Koch T, Wahlestedt C (2005) Locked nucleic acid (LNA) mediated improvements in siRNA stability and functionality. Nucleic Acids Res 33:439-447.
Fellmann C, Hoffmann T, Sridhar V, Hopfgartner B, Muhar M, Roth M, Lai D Y, Barbosa I A, Kwon J S, Guan Y, Sinha N, Zuber J; An Optimized microRNA Backbone for Effective Single-Copy RNAi; Cell Rep. 2013 Dec. 26; 5(6):1704-13
Gimenez A, Storrer K, Kuranishi L, et al. Change in FVC and survival in chronic fibrotic hypersensitivity pneumonitis. Thorax 2017; 73:391-392
Goh N S, Hoyles R K, Denton C P, et al. Short-term pulmonary function trends are predictive of mortality in interstitial lung disease associated with systemic sclerosis. Arthritis Rheumatol 2017; 69:1670-1678
Grimson MicroRNA Targeting Specificity in Mammals: Determinants beyond Seed Pairing, Volume 27, Issue 1, 6 Jul. 2007, Pages 91-105, https://doi.org/10.1016/j.molcel.2007.06.017
Guler S A, Winstone T A, Murphy D, et al. Does systemic-sclerosis-associated interstitial lung disease burn out? Specific phenotypes of disease progression. Ann Am Thorac Soc 2018; 15(12):1427-1433
Fischer A, Brown K K, du Bois R M et al. Mycophenolate mofetil improves lung function in connective tissue disease-associated interstitial lung disease. J Rheumatol. 2013; 40:640-6
Flaherty K R, Brown K K, Wels A U, et al. Design of the P F-ILD trial: a double-blind, randomised, placebo-controlled phase III trial of nintedanib in patients with progressive fibrosing interstitial lung disease. BMJ Open Respir Res 2017; 4:e000212
Freier, S. The ups and downs of nucleic acid duplex stability: structure-stability studies on chemically-modified DNA:RNA duplexes, Nucleic Acids Research (volume 25 issue 22 pages 4429-4443
Galié N, Humbert M, Vachiery J L, et al. 2015 ESC/ERS Guidelines for the diagnosis and treatment of pulmonary hypertension. ERJ 2015 46: 903-975; DOI: 10.1183/13993003.01032-2015
Guler S A, Ellison K, Algamdi M, Collard H R, Ryerson C J. Heterogeneity in unclassifiable interstitial lung disease: a systematic review and meta-analysis. Ann Am Thorac Soc 2018; 15(7):854-863
Hall, A., RNA interference using boranophosphate siRNAs: structure-activity relationships, Nucleic Acids Research, 2004, Vol. 32, No. 20 5991-6000
Hopkins R B, Burke N, Fell C, Dion G, Kolb M; Epidemiology and survival of idiopathic pulmonary fibrosis from national data in Canada; Eur. Respir. J., 48 (1) (2016), pp. 187-195
Jegal Y, Kim D S, Shim T S et al. Physiology is a stronger predictor of survival than pathology in fibrotic interstitial pneumonia. Am J Respir Crit Care Med 2005; 171:639-644
Jiang, R., The emerging roles of a novel CCCH-type zinc finger protein, ZC3H4, in silica-induced epithelial to mesenchymal transition, Toxicology Letters 307 (2019) 26-40
Khalil N, Churg A, Muller N, O'Connor R. Environmental, inhaled and ingested causes of pulmonary fibrosis. Toxicol Pathol 2007; 35:86-96
Kim M Y, Song J W, Do K H, et al. Idiopathic nonspecific interstitial pneumonia: changes in high-resolution computed tomography on long-term follow-up. J Comput Assist Tomogr 2012; 36:170-174
Kolb M, Vasakova M. The natural history of progressive fibrosing interstitial lung diseases. Resp Res (Lond) 2019; 20(1):57
Körbelin J, Sieber T, Michelfelder S, Lunding L, Spies E, Hunger A, Alawi M, Rapti K, Indenbirken D, Müller O J, Pasqualini R, Arap W, Kleinschmidt J A, Trepel M; Pulmonary Targeting of Adeno-associated Viral Vectors by Next-generation Sequencing-guided Screening of Random Capsid Displayed Peptide Libraries; Mol Ther. 2016 June; 24(6):1050-1061
Langfelder P, Horvath S; WGCNA: an R package for weighted correlation network analysis; BMC Bioinformatics. 2008 Dec. 29; 9:559
Ley B, Collard H R (2013); Epidemiology of idiopathic pulmonary fibrosis; Clin. Epidemiol., 5:483-492
Liao Y, Smyth G K, Shi W; feature Counts: an efficient general purpose program for assigning sequence reads to genomic features; Bioinformatics 30, 923-930 (2014)
Limberis M P, Vandenberghe L H, Zhang L, Pickles R J, Wilson J M; Transduction efficiencies of novel AAV vectors in mouse airway epithelium in vivo and human ciliated airway epithelium in vitro; Mol Ther. 2009 February;17(2):294-301
Luckhardt T R, Thannickal V J. Systemic sclerosis-associated fibrosis: an accelerated aging phenotype? Curr Opin Rheumatol 2015; 27:571-576
Mathai S C, Danoff S K; Management of interstitial lung disease associated with connective tissue disease; BMJ. 2016 Feb. 24; 352
Mook O R, Baas F, de Wissel M B, Fluiter K (2007) Evaluation of locked nucleic acid-modified small interfering RNA in vitro and in vivo. Mol Cancer Ther 6:833-843.
Morisset J, Johannson K A, Vittinghoff E et al. Use of mycophenolate mofetil or azathioprine for the management of chronic hypersensitivity pneumonitis. Chest. 2017; 151:619-625
Naso, W F, Tomkowicz, B, Perry W L, Strohl, W Adeno-Associated Virus (AAV) as a Vector for Gene Therapy; BioDrugs (2017) 31:317-334
Osborn, M., Improving siRNA Delivery In Vivo Through Lipid Conjugation, Nucleic Acid Therapeutics, Volume 28, Number 3, 2018
Pajak M and Simpson T I (2016); miRNAtap: miRNAtap: microRNA Targets—Aggregated Predictions. R package version 1.10.0.
Rajwanshi. V, The eight stereoisomers of LNA (locked nucleic acid): a remarkable family of strong RNA binding molecules, Angewandte Chemie, International Edition Volume 39, Issue 9 Pages 1656-1659 Journal 2000
Raghu G, Collard H R, Egan J J, et al.; ATS/ERS/JRS/ALAT Committee on Idiopathic Pulmonary Fibrosis An official ATS/ERS/JRS/ALAT statement: idiopathic pulmonary fibrosis: evidence-based guidelines for diagnosis and management; Am J Respir Crit Care Med. 2011; 183(6):788-824
Ritchie M E, Phipson B, Wu D, Hu Y, Law C W, Shi W, Smyth G K; limma powers differential expression analyses for RNA-sequencing and microarray studies; Nucleic Acids Res. 2015 Apr. 20; 43(7)
Sadeleer L J de, Hermans F, Dycker E de, et al. Effects of corticosteroid treatment and antigen avoidance in a large hypersensitivity pneumonitis cohort: a single-centre cohort study. J Clin Med 2019; 8:14
Sayols S, Scherzinger D, Klein H; dupRadar: a Bioconductor package for the assessment of PCR artifacts in RNA-Seq data; BMC Bioinformatics 17, 428 (2016)
Scott M. Hammond; An overview of microRNAs; Adv Drug Deliv Rev. 2015 Jun. 29; 87: 3-14
Schwartz M I, King T E. Interstitial lung disease 5th Ed. Shelton: People's Medical Publishing House 2011
Solomon J J, Chung J H, Cosgrove G P, et al. Predictors of mortality in rheumatoid arthritis-associated interstitial lung disease. Eur Respir J 2016; 47:588-596
Spagnalo P, Rossi G, Trisolini R, Sverzellati N, Baughman R P, Wells A U. Pulmonary sarcoidosis. Lancet Respir Med. 2018 May; 6(5):389-402.
Strobel B, Duechs M J, Schmid R, Stierstorfer B E, Bucher H, Quast K, Stiller D, Hildebrandt T, Mennerich D, Gantner F, Erb K J, Kreuz S; Modeling Pulmonary Disease Pathways Using Recombinant Adeno-Associated Virus 6.2; Am J Respir Cell Mol Biol. 2015 September;53(3):291-302.
Strobel B, Miller F D, Rist W, Lamla T. Comparative Analysis of Cesium Chloride- and Iodixanol-Based Purification of Recombinant Adeno-Associated Viral Vectors for Pre-clinical Applications; Hum Gene Ther Methods. 2015 August; 26(4):147-57
Strobel, B. Modeling pulmonary fibrosis by AAV-mediated TGFβ1 Expression: a proof of concept study for AAV-based disease modeling and riboswitch-controlled vector production; Konstanz, Univ., Diss., 2016, 2018, http://kops.unikonstanz.de/handle/123456789/33826
Sun, S Enhancing the Therapeutic Delivery of Oligonucleotides by Chemical Modification and Nanoparticle Encapsulation, Molecules 2017, 22, 1724; doi:10.3390/molecules22101724
Thannickal V J, Zhou Y, Gaggar A, Duncan S R. Fibrosis: ultimate and proximate causes. J Clin Invest 2014; 124(11):4673-4677
Tashkin D P, Elashoff R, Clements P J et al. Scleroderma Lung Study Research Group. Cyclophosphamide versus placebo in scleroderma lung disease. N Engl J Med. 2006; 354:2655-66
Uhlmann, Recent advances in the medicinal chemistry of antisense oligonucleotides, Curr Opinion in Drug Development, vol. 3, no. 2, 2000, pages 203-213
Vinnikov, I. A., Hypothalamic miR-103 Protects from Hyperphagic Obesity in Mice, The Journal of Neuroscience, Aug. 6, 2014·34(32):10659-10674·10659
Volkmann E R, Tashkin D P, Sim M, Kim G H, Goldin J, Clements P J. Determining progression of scleroderma-related interstitial lung disease. J Scleroderma Rel Disord 2019; 4(1):62-70
Walsh S L, Wells A U, Sverzellati N, et al. An integrated clinicoradiological staging system for pulmonary sarcoidosis: a case-cohort study. Lancet Respir Med 2014; 2:123-30
Wells A U. Approach to diagnosis of diffuse lung disease. Clinical respiratory medicine 2nd Ed. Albert R K, Spiro S G & Jett J R (Eds.). Mosby [Elsevier Science] 2004
Wells A U, Brown K K, Flaherty K R, Kolb M, Thannickal V J on behalf of the IPF Consensus Working Group. What's in a name? That which we call IPF, by any other name would act the same. Eur Respir J 2018; 51:1800692
Wollin L, Distler J H W, Redente E F, et al. Potential of nintedanib in treatment of progressive fibrosing interstitial lung diseases. Eur Respir J 2019; 54(3). pii: 1900161
Wu Z, Asokan A, Grieger J C, Govindasamy L, Agbandje-McKenna M, Samulski R J Single amino acid changes can influence titer, heparinbinding, and tissue tropism in different adeno-associated virus serotypes; J Virol 2006; 80:11393-11397
Xianbin, Y., Gene silencing activity of siRNA molecules containing phosphorodithioate substitutions ACS Chem. Biol. 2012, 7, 1214-1220
Yang, X., Silica-induced initiation of circular ZC3H4 RNA/ZC3H4 pathway promotes the pulmonary macrophage activation. FASEB J. 32, 3264-3277 (2018). www.faseb*.org

TABLE 1
Sequence listing index

| Seq ID | |
|---|---|
| NO: | Description |
| 1-40 | See FIG. 5A and 5B |
| 40 | mir-10a-5p 23 nt, miR-E backbone, Passenger position |
| 41 | mir-10a-5p 23 nt, miR-E backbone, Guide position |
| 42 | mir-10a-5p 22 nt, miR-E backbone, Passenger position |
| 43 | mir-10a-5p 22 nt, miR-E backbone, Guide position |
| 44 | mir-10a-5p, natural pre-miRNA in miR-E backbone, Human (hsa-mir-10a MI0000266) |
| 45 | mir-10a-5p, natural pre-miRNA in miR-E backbone, Mouse (mmu-mir-10a MI0000685) |
| 46 | mir-181a-5p 23 nt, miR-E backbone, Passenger position |
| 47 | mir-181a-5p 23 nt, miR-E backbone, Guide position |
| 48 | mir-181a-5p 22 nt, miR-E backbone, Passenger position |
| 49 | mir-181a-5p 22 nt, miR-E backbone, Guide position |
| 50 | mir-181a-5p, natural pre-miRNA in miR-E backbone, Human (hsa-mir-181a-1 |
| | MI0000289) |
| 51 | mir-181a-5p, natural pre-miRNA in miR-E backbone, Mouse (mmu-mir-181a-1 |
| | MI0000697) |
| 52 | mir-181b-5p 23 nt, miR-E backbone, Passenger position |
| 53 | mir-181b-5p 23 nt, miR-E backbone, Guide position |
| 54 | mir-181b-5p 22 nt, miR-E backbone, Passenger position |
| 55 | mir-181b-5p 22 nt, miR-E backbone, Guide position |
| 56 | mir-181b-5p, natural pre-miRNA in miR-E backbone, Human (hsa-mir-181b-1 |
| | MI0000270) |
| 57 | mir-181b-5p, natural pre-miRNA in miR-E backbone, Mouse (mmu-mir-181b-1 |
| | MI0000723) |
| 58 | mir-212-5p 23 nt, miR-E backbone, Passenger position |
| 59 | mir-212-5p 23 nt, miR-E backbone, Guide position |
| 60 | mir-212-5p 22 nt, miR-E backbone, Passenger position |
| 61 | mir-212-5p 22 nt, miR-E backbone, Guide position |
| 62 | mir-212-5p, natural pre-miRNA in miR-E backbone, Human (hsa-mir-212 MI0000288) |
| 63 | mir-212-5p, natural pre-miRNA in miR-E backbone, Mouse (mmu-mir-212 MI0000696) |
| 64 | scAAV-CMV-eGFP-mir181b-5p(23 nt in miR-E backbone)-SV40pA, Passenger position |
| 65 | scAAV-CMV-eGFP-mir181b-5p(23 nt in miR-E backbone)-SV40pA, Guide position |
| 66 | scAAV-CMV-eGFP-mir181b-5p(22 nt in miR-E backbone)-SV40pA, Passenger position |
| 67 | scAAV-CMV-eGFP-mir181b-5p(22 nt in miR-E backbone)-SV40pA, Guide position |
| 68 | scAAV-CMV-eGFP-mir181b-5p(natural pre-miRNA, human)-SV40pA |
| 69 | scAAV-CMV-eGFP-mir181b-5p(natural pre-miRNA, mouse)-SV40pA |
| 70 | scAAV-CMV-eGFP-mir-181a-mir181b-mir10a(all 23 nt in miR-E backbone), Passenger |
| | position |
| 71 | scAAV-CMV-eGFP-mir-181a-mir181b-mir10a(all 23 nt in miR-E backbone), Guide |
| | position |
| 72 | scAAV-CMV-eGFP-mir-181a-mir181b-mir10a(all 22 nt in miR-E backbone), Passenger |
| | position |
| 73 | scAAV-CMV-eGFP-mir-181a-mir181b-mir10a(all 22 nt in miR-E backbone), Guide |
| | position |
| 74 | scAAV-CMV-eGFP-mir-212-5p-mir181b-mir10a(all 23 nt in miR-E backbone), Passenger |
| | position |
| 75 | scAAV-CMV-eGFP-mir-212-5p-mir181b-mir10a(all 23 nt in miR-E backbone), Guide |
| | position |
| 76 | scAAV-CMV-eGFP-mir-212-5p-mir181b-mir10a(all 22 nt in miR-E backbone), Passenger |
| | position |
| 77 | scAAV-CMV-eGFP-mir-212-5p-mir181b-mir10a(all 22 nt in miR-E backbone), Guide |
| | position |
| 78 | scAAV-CMV-eGFP-mir-181a-mir181b-mir10a(natural pre-miRNAs in miR-E backbone), |
| | Human |
| 79 | scAAV-CMV-eGFP-mir-181a-mir181b-mir10a(natural pre-miRNAs in miR-E backbone), |
| | Mouse |
| 80 | scAAV-CMV-eGFP-mir-212-5p-mir181b-mir10a(natural pre-miRNAs in miR-E |
| | backbone), Human |
| 81 | scAAV-CMV-eGFP-mir-212-5p-mir181b-mir10a(natural pre-miRNAs in miR-E |
| | backbone), Mouse |
| 83 | mir-Ren713, neutral control, miR-E backbone |
| 84 | mir-181a stem-loop, miR-E context, Human |
| 85 | mir-212 stem-loop, miR-E context, Human |
| 86 | mir-29a-3p, miR-E backbone |
| 87 | mir-181a stem-loop, miR-E context, Mouse |
| 88 | mir-212 stem-loop, miR-E context, Mouse |
| 89 | mir-29a stem-loop, miR-E context |
| 90 | mir-29a, natural pre-miRNA in miR-E backbone |
| 91 | pAAVsc_CMV-miR212-5p, 22 nt, in mir-E backbone, circular, plasmid |
| 92 | hsa miR-29a-3p |
| 93 | mir-29a stem loop-mir181a stem loop-bGH pA |
| 94 | mir-29a stem loop-mir212 stem loop-bGH pA |
| 95 | mir-29a-3p-mir-181a-5p 23 nt, miR-E backbone, Guide position, bGH pA |
| 96 | mir-29a-3p-mir-181a-5p 22 nt, miR-E backbone, Guide position, bGH pA |
| 97 | mir-29a-3p-mir-212-5p 23 nt, miR-E backbone, Guide position, bGH pA |
| 98 | mir-29a-3p-mir-212-5p 22 nt, miR-E backbone, Guide position, bGH pA |
| 99 | hsa-mir-212-5p sequence as published in mirBase as MIMAT0022695 without the last |
| | nucleotide at the 3′ terminus 22 nt |
| 100 | hsa-mir-181a-5p sequence as published in mirBase as MIMAT0000256 without the last |
| | nucleotide at the 3′ terminus 22 nt |

1. Viral vector comprising: a capsid and a packaged nucleic acid, wherein the packaged nucleic acid codes for two or more miRNAs, wherein the two or more miRNAs comprise the miRNA of Seq ID No. 92 and the miRNA of Seq ID No. 15 or a fragment thereof having the sequence of Seq ID No. 99.

2. Viral vector comprising: a capsid and a packaged nucleic acid, wherein the packaged nucleic acid codes for two or more miRNAs, wherein the two or more miRNAs comprise the miRNA of Seq ID No. 92 and the miRNA of Seq ID No. 17 or a fragment thereof having the sequence of Seq ID No. 100.

3. Viral vector according to claim 1 or 2, wherein the packaged nucleic acid codes for more than two miRNA, wherein said miRNAs comprise (i) the miRNA of Seq ID No. 92 and (ii) the miRNA of Seq ID No. 15 or a fragment thereof having the sequence of Seq ID No. 99 and (iii) the miRNA of Seq ID No. 17 or a fragment thereof having the sequence of Seq ID No. 100.

4. Viral vector according to claim 3, wherein the packaged nucleic acid codes for a miRNA having the sequence of Seq ID No. 92, and for a miRNA having the sequence of Seq ID No. 15 and for a miRNA having the sequence of Seq ID No. 17.

5. Viral vector according to any of claims 1-4, comprising: a capsid and a packaged nucleic acid comprising one or more transgene expression cassettes comprising a transgene that codes
for the miRNA of Seq ID No. 92 and at least one of the miRNAs selected from the group consisting of Seq ID No. 15 or a fragment thereof having the sequence of Seq ID No. 99 and Seq ID No. 17 or a fragment thereof having the sequence of Seq ID No. 100, and
for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

6. Viral vector according to any of claims 1-5, comprising: a capsid and a packaged nucleic acid comprising two or more transgene expression cassettes comprising a transgene,
wherein the first expression cassette comprises a first transgene that codes for the miRNA of Seq ID No. 92 and at least one of the miRNAs selected from the group consisting of Seq ID No. 15 or a fragment thereof having the sequence of Seq ID No. 99 and Seq ID No. 17 or a fragment thereof having the sequence of Seq ID No. 100, and
wherein the second expression cassette comprises a second transgene that codes for an RNA that inhibits the function of one or more miRNAs selected form the group consisting of miRNAs of Seq ID No 1, 2, 3, 4, 5, 6, 7, 8, 9, 10, 11, 12, 13, 14, 16, 34, 35 and 36.

7. Viral vector according to one of claims 5 to 6, wherein the inhibiting RNA is not subject to RNAi processing or RNAi maturation.

8. Viral vector according to one of claims 5 to 7, wherein the nucleic acid has an even number of transgene expression cassettes.

9. Viral vector according to anyone of claims 5 to 8, wherein the transgene expression cassettes comprise a promotor, a transgene and a polyadenylation signal, wherein promotors or the polyadenylation signals are positioned opposed to each other.

10. Viral vector according to anyone of claim 1 to 9, wherein the vector is a recombinant AAV vector.

11. Viral vector according to anyone of claim 1 to 10, wherein the vector is a recombinant AAV vector having the AAV-2 serotype.

12. Viral vector according to anyone of claim 1 to 11, wherein the capsid comprises a first protein that comprises the sequence of Seq ID No. 29 or 30.

13. Viral vector according to anyone of claim 1 to 12, wherein the capsid comprises a first protein that is 80% identical to a second protein having the sequence of Seq ID No. 82, whereas one or more gaps in the alignment between the first protein and the second protein are allowed.

14. Viral vector according to anyone of claim 1 to 13, wherein the capsid comprises a first protein that is 95% identical to a second protein of Seq ID No. 82, whereas a gap in the alignment between the first protein and the second protein is counted as a mismatch.

15. Viral vector according to anyone of claim 1 to 14, wherein the vector is a recombinant AAV vector having the AAV5 or the AAV6.2 serotype, and wherein the capsid of the recombinant AAV6.2 vector preferably comprises a capsid protein having the sequence of Seq ID No. 82.

16. Viral vector according to anyone of claim 1 to 15, wherein packaged nucleic acid is double-stranded.

17. Viral vector according to anyone of claim 1 to 15, wherein packaged nucleic acid is single-stranded.

18. Viral vector according to anyone of claims 1 to 17 for use in the prevention or treatment of a disease selected from the group consisting of ILD, PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, pulmonary hypertension (PH), fibrotic silicosis, systemic sclerosis ILD, sarcoidosis, and fibrosarcoma.

19. Method of treating a disease selected from the group consisting of ILD, PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, pulmonary hypertension (PH), fibrotic silicosis, systemic sclerosis ILD, sarcoidosis, and fibrosarcoma, the method comprising administering to a patient in need thereof a therapeutically active amount of viral vector according to anyone of claims 1 to 17.

20. Viral vector according to anyone of claims 1 to 17 for use as a medicinal product.

21. AAV vector comprising a vector genome that codes for two or more miRNAs, wherein the two or more miRNAs comprise the miRNA of Seq ID No. 92 and the miRNA of Seq ID No. 15 or a fragment thereof having the sequence of Seq ID No. 99.

22. AAV vector comprising a vector genome that codes for two or more miRNAs, wherein the two or more miRNAs comprise the miRNA of Seq ID No. 92 and the miRNA of Seq ID No. 17 or a fragment thereof having the sequence of Seq ID No. 100.

23. AAV vector according to claim 21 or 22, wherein said vector genome codes for (i) a miRNA comprising the sequence of Seq ID No. 92 and (ii) for a miRNA comprising the sequence of Seq ID No. 15 or a fragment thereof having the sequence of Seq ID No. 99, and (iii) for a miRNA comprising the sequence of Seq ID No. 17 or a fragment thereof having the sequence of Seq ID No. 100.

24. AAV vector according to claim 23, wherein said vector genome codes for (i) a miRNA having the sequence of Seq ID No. 92 and (ii) for a miRNA having the sequence of Seq ID No. 15 and (iii) for a miRNA having the sequence of Seq ID No. 17.

25. AAV vector according to any of claims 21 to 24, wherein said vector genome further codes for an RNA that inhibits the function of one or more miRNAs selected form the group consisting of the miRNAs of Seq ID Nos. 1, 2, 3, 4, 5, 6, 7, 8, 9, 10, 11, 12, 13, 14, 16, 34, 35 and 36.

26. Double-stranded plasmid vector comprising an AAV vector of any of claims 21 to 25.

27. A combination of miRNA mimetics for use in a method of prevention and/or treatment of a fibroproliferative disorder, wherein the combination comprises (i) a mimetic of the miRNA having the sequence of Seq ID No. 92, and (ii) a mimetic of the miRNA having the sequence of Seq ID No. 15 and/or a mimetic of the miRNA having the sequence of Seq ID No. 17.

28. A miRNA mimetic of miRNA-29a-3p for use in a method of prevention and/or treatment of a fibroproliferative disorder, wherein the miRNA mimetic is or contains an oligomer of nucleotides that consist of the sequence of Seq ID No. 92, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 92;
the oligomer optionally comprises nucleotide analogues that show the basepairing behavior at the corresponding position (AU and GC) as determined by the sequence of Seq ID No. 92;
the oligomer is optionally lipid conjugated to facilitate drug delivery,
wherein said prevention and/or treatment further comprises the administration of a mimetic of a miRNA having the sequence of Seq ID No. 15 and/or a mimetic of a miRNA having the sequence of Seq ID No. 17.

29. A miRNA mimetic for use in a method according to claim 28, wherein said prevention and/or treatment further the administration of a mimetic of a miRNA having the sequence of Seq ID No. 15.

30. A miRNA mimetic for use in a method according to claim 29, wherein the mimetic of a miRNA having the sequence of Seq ID No. 15 is or contains an oligomer of nucleotides that consists of the sequence of Seq ID No. 15 or Seq ID No. 99, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15 or Seq ID No. 99;
the oligomer optionally comprises nucleotide analogues that show the basepairing behavior at the corresponding position (AU and GC) as determined by the sequence of Seq ID No. 15 or Seq ID No. 99;
the oligomer is optionally lipid conjugated to facilitate drug delivery.

31. A miRNA mimetic for use in a method according to claim 28, wherein said prevention and/or treatment further comprises the administration of a mimetic of a miRNA having the sequence of Seq ID No. 17.

32. A miRNA mimetic for use in a method according to claim 31, wherein the mimetic of a miRNA having the sequence of Seq ID No. 17 is or contains an oligomer of nucleotides that consists of the sequence of Seq ID No. 17 or Seq ID No. 100, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 17 or Seq ID No. 100;
the oligomer optionally comprises nucleotide analogues that show the basepairing behavior at the corresponding position (AU and GC) as determined by the sequence of Seq ID No. 17 or Seq ID No. 100;
the oligomer is optionally lipid conjugated to facilitate drug delivery.

33. A miRNA mimetic for use in a method according to anyone of claims 28 to 32, wherein said prevention and/or treatment further comprises the administration of a mimetic of a miRNA having the sequence of Seq ID No. 19.

34. A miRNA mimetic for use in a method according to claim 33, wherein the mimetic of a miRNA having the sequence of Seq ID No. 19 is or contains an oligomer of nucleotides that consists of the sequence of Seq ID No. 19, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 19;
the oligomer optionally comprises nucleotide analogues that show the basepairing behavior at the corresponding position (AU and GC) as determined by the sequence of Seq ID No. 19;
the oligomer is optionally lipid conjugated to facilitate drug delivery.

35. A miRNA mimetic for use in a method according to any of claims 28 to 34, wherein the fibroproliferative disorder is IPF or PF-ILD.

36. Use of (i) a miRNA mimetic of a miRNA having the sequence of Seq ID No. 92 and (ii) a miRNA mimetic of a miRNA having the sequence of Seq ID No. 15 and/or a miRNA mimetic of a miRNA having the sequence of Seq ID No. 17 for the manufacture of a medicament for the treatment of a fibroproliferative disorder such as IPF or PF-ILD or ILD.

37. Pharmaceutical composition comprising a miRNA mimetic of a miRNA having the sequence of Seq ID No. 92 and a miRNA mimetic of a miRNA having the sequence of Seq ID No. 15, and a pharmaceutical-acceptable carrier or diluent.

38. Pharmaceutical composition comprising a miRNA mimetic of a miRNA having the sequence of Seq ID No. 92 and a miRNA mimetic of a miRNA having the sequence of Seq ID No. 17, and a pharmaceutical-acceptable carrier or diluent.

39. Pharmaceutical composition comprising a miRNA mimetic of a miRNA having the sequence of Seq ID No. 92 and a miRNA mimetic of a miRNA having the sequence of Seq ID No. 17, and a miRNA mimetic of a miRNA having the sequence of Seq ID No. 15, and a pharmaceutical-acceptable carrier or diluent.

40. Pharmaceutical composition according to claim 37, 38, or 39, wherein the miRNA mimetics in said composition are packed in lipid nanoparticles (LNPs).

41. Pharmaceutical composition according to claim 40, wherein said composition comprises to 65 mol % of ionizable lipids.

42. Pharmaceutical composition according to any one of claims 37-41, wherein the mean particle size of the LNPs is between 30 and 200 nm.

43. Pharmaceutical composition comprising
(a) a miRNA mimetic of miRNA 29a-3p, wherein the miRNA mimetic is or contains an oligomer of nucleotides that consists of the sequence of Seq ID No. 92, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 92;
the oligomer optionally comprises nucleotide analogues that show the basepairing behavior at the corresponding position (AU and GC) as determined by the sequence of Seq ID No. 92;
the oligomer is optionally lipid conjugated to facilitate drug delivery; and
(b) a miRNA mimetic of miRNA 212-5p, wherein the miRNA mimetic is or contains an oligomer of nucleotides that consists of the sequence of Seq ID No. 15 or Seq ID No. 99, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15 or Seq ID No. 99;
the oligomer optionally comprises nucleotide analogues that show the basepairing behavior at the corresponding position (AU and GC) as determined by the sequence of Seq ID No. 15 or Seq ID No. 99,
the oligomer is optionally lipid conjugated to facilitate drug delivery; and
(c) a pharmaceutical-acceptable carrier or diluent.

44. Pharmaceutical composition comprising
(a) a miRNA mimetic of miRNA 29a-3p, wherein the miRNA mimetic is or contains an oligomer of nucleotides that consist of the sequence of Seq ID No. 92, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 92;
the oligomer optionally comprises nucleotide analogues that show the basepairing behavior at the corresponding position (AU and GC) as determined by the sequence of Seq ID No. 92; and
the oligomer is optionally lipid conjugated to facilitate drug delivery, and
(b) a miRNA mimetic of miRNA 181a-5p, wherein the miRNA mimetic is or contains an oligomer of nucleotides that consist of the sequence of Seq ID No. 17 or Seq ID No. 100, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 17 or Seq ID No. 100;
the oligomer optionally comprises nucleotide analogues that show the basepairing behavior at the corresponding position (AU and GC) as determined by the sequence of Seq ID No. 17 or Seq ID No. 100,
the oligomer is optionally lipid conjugated to facilitate drug delivery; and
(c) a pharmaceutical-acceptable carrier or diluent.

45. Pharmaceutical composition comprising
(a) a miRNA mimetic of miRNA 29a-3p, wherein the miRNA mimetic is or contains an oligomer of nucleotides that consist of the sequence of Seq ID No. 92, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 92;
the oligomer optionally comprises nucleotide analogues that show the basepairing behavior at the corresponding position (AU and GC) as determined by the sequence of Seq ID No. 92; and
the oligomer is optionally lipid conjugated to facilitate drug delivery, and
(b) a miRNA mimetic of miRNA 181a-5p, wherein the miRNA mimetic is or contains an oligomer of nucleotides that consist of the sequence of Seq ID No. 17 or Seq ID No. 100, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 17 or Seq ID No. 100;
the oligomer optionally comprises nucleotide analogues that show the basepairing behavior at the corresponding position (AU and GC) as determined by the sequence of Seq ID No. 17 or Seq ID No. 100,
the oligomer is optionally lipid conjugated to facilitate drug delivery; and
(c) a miRNA mimetic of miRNA 212-5p, wherein the miRNA mimetic is or contains an oligomer of nucleotides that consists of the sequence of Seq ID No. 15 or Seq ID No. 99, with the following proviso:
the oligomer optionally comprises nucleotides with chemical modifications leading to non-naturally occurring nucleotides that show the base-pairing behavior at the corresponding position (AU and GC) as determined by the sequence of Seq ID No. 15 or Seq ID No. 99;
the oligomer optionally comprises nucleotide analogues that show the basepairing behavior at the corresponding position (AU and GC) as determined by the sequence of Seq ID No. 15 or Seq ID No. 99,
the oligomer is optionally lipid conjugated to facilitate drug delivery; and
(d) a pharmaceutical-acceptable carrier or diluent.

46. The pharmaceutical composition according to any of claim of 37 to 44, wherein the miRNA mimetic of miRNA 29a-3p is a double-strand miRNA mimetic.

47. The pharmaceutical composition according to claim 37, 39, 40, 41, 43 or 45, wherein the miRNA mimetic of miRNA-212-5p is a double-strand miRNA mimetic.

48. The pharmaceutical composition according to claim 38, 39, 40, 42, 44 or 45, wherein the miRNA mimetic of miRNA-181a-5p is a double-strand miRNA mimetic.

49. Method of treating a disease selected from the group consisting of ILD, PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, pulmonary hypertension (PH), fibrotic silicosis, systemic sclerosis ILD, sarcoidosis, and fibrosarcoma, the method comprising administering to a patient in need thereof a therapeutically active amount of a pharmaceutical composition according to any of claims 37 to 48.

50. Use of a pharmaceutical composition according to any of claims 37 to 48 for the manufacture of a medicament for the treatment of a disease selected from the group consisting of ILD, PF-ILD, IPF, connective tissue disease (CTD)-associated ILD, rheumatoid arthritis ILD, chronic fibrosing hypersensitivity pneumonitis (HP), idiopathic non-specific interstitial pneumonia (iNSIP), unclassifiable idiopathic interstitial pneumonia (IIP), environmental/occupational lung disease, pulmonary hypertension (PH), fibrotic silicosis, systemic sclerosis ILD, sarcoidosis, and fibrosarcoma.
